(19) United States
(12) Patent Application Publication
(10) Pub. No.: US 2024/0181038 A1
(43) Pub. Date: Jun. 6, 2024

(54) IMMUNOGENIC COMPOSITIONS

(71) Applicants: GLAXOSMITHKLINE BIOLOGICALS SA, Rixensart (BE); UNITED STATES OF AMERICA, as represented by THE SECRETARY, DEPARTMENT OF HEALTH AND HUMAN SERVICES, Bethesda, MD (US)

(72) Inventors: Ventzislav Bojidarov VASSILEV, Rixensart (BE); Corey MALLETT, Rockville, MD (US); Ronan ROUXEL, Rixensart (BE); Normand BLAIS, Rixensart (BE); Masaru KANEKIYO, Bethesda, MD (US); Barney S. GRAHAM, Bethesda, MD (US)

(73) Assignees: GLAXOSMITHKLINE BIOLOGICALS SA, Rixensart (BE); UNITED STATES OF AMERICA, as represented by THE SECRETARY, DEPARTMENT OF HEALTH AND HUMAN SERVICES, Bethesda, MD (US)

(21) Appl. No.: 18/283,632

(22) PCT Filed: Mar. 25, 2022
(86) PCT No.: PCT/EP2022/057933

§ 371 Date: Sep. 22, 2023

Related U.S. Application Data
(60) Provisional application No. 63/166,539, filed on Mar. 26, 2021.

(51) Int. Cl. A61K 39/145; A61K 39/00; A61K 39/385; A61P 31/16; C07K 14/005

(52) U.S. Cl. CPC A61K 39/145; A61K 39/385; A61P 31/16; C07K 14/005; A61K 2039/53; A61K 2039/55555; C12N 2760/16122; C12N 2760/16134

(57) ABSTRACT

The present invention relates to a carrier-formulated mRNA comprising at least one coding sequence encoding an influenza HA stem polypeptide, and to related aspects.

The present application claims priority to U.S. provisional application No. 63/166,539 filed on Mar. 26, 2021, the contents of which are incorporated by reference in their entirety.

This invention was created in the performance of a Cooperative Research and Development Agreement with the National Institutes of Health, an Agency of the Department of Health and Human Services. The Government of the United States has certain rights in this invention.

### TECHNICAL FIELD

The present invention relates to influenza immunisation using a hemagglutinin (HA) stem polypeptide delivered in the form of carrier-formulated mRNA, and to related aspects.

### BACKGROUND

Influenza viruses have a significant impact on global public health, causing millions of cases of severe illness each year, thousands of deaths, and considerable economic losses. Current tri- or tetravalent influenza vaccines elicit antibody responses to the vaccine strains and closely related isolates, but rarely extend to more diverged strains within a subtype or to other subtypes. In addition, selection of the appropriate vaccine strains presents many challenges and frequently results in sub-optimal protection.

Protective immune responses induced by vaccination against influenza viruses are primarily directed to the viral HA protein, which is a glycoprotein on the surface of the virus responsible for interaction of the virus with host cell receptors. HA proteins on the virus surface are trimers of HA protein monomers that are enzymatically cleaved to yield amino-terminal HA1 and carboxy-terminal HA2 polypeptides. The globular head consists exclusively of the major portion of the HA1 polypeptide, whereas the stem that anchors the HA protein into the viral lipid envelope is comprised of HA2 and part of HA1. The globular head of a HA protein includes two domains: the receptor binding domain (RBD), a domain that includes the sialic acid-binding site, and the vestigial esterase domain, a smaller region just below the RBD. The globular head includes several antigenic sites that include immunodominant epitopes.

Therefore, antibodies against influenza often target variable antigenic sites in the globular head of HA and thus, neutralize only antigenically closely related viruses. The variability of the HA head is due to the constant antigenic drift (i.e., changes in the protein sequence) of influenza viruses and is responsible for seasonal endemics of influenza. Based on the sequence of HA and that of the other surface glycoprotein neuraminidase (NA), which is also affected by antigenic drift, influenza virus strains are classified into different subtypes. In total, 18 HAs and 11 NAs have been isolated thus far and are further divided into two groups each, e.g. HA group 1 contains e.g. H1, H2, H5, and H9 and group 2 contains e.g. H3, H7, and H10.

In contrast to the HA-head, the HA stem is highly conserved and experiences little antigenic drift.

In fact, an entirely new class of broadly neutralizing antibodies against influenza viruses has been isolated that recognize the highly conserved HA stem (Corti, 2011). Unlike strain-specific antibodies, antibodies in this new class are capable of neutralizing multiple antigenically distinct viruses. However, robustly eliciting these antibodies in subjects by vaccination with the HA stem, lacking the head domain, has been difficult (Steel, 2010). Removal of the immunodominant head region of HA (which contains competing epitopes) and stabilization of the resulting stem region through genetic manipulation is one potential way to improve the elicitation of these broadly neutralizing stem antibodies.

Advances in biotechnology in past decades have allowed engineering of biological materials to be exploited for the generation of novel vaccine platforms. Ferritin, an iron storage protein found in almost all living organisms, is an example which has been extensively studied and engineered for a number of potential biochemical/biomedical purposes. The use of ferritin self-assembling nanoparticles to present stabilised stem trimers is described in Corbett, 2019.

Messenger RNA (mRNA) is a single-stranded RNA molecule that corresponds to the genetic sequence of a gene and is read by ribosomes in the process of producing a protein. mRNA based vaccines provide an alternative vaccination approach to traditional strategies involving live attenuated/inactivated pathogens or subunit vaccines (Zhang, 2019). mRNA vaccines may utilise non-replicating mRNA or self-replicating RNA (also referred to as self-amplifying mRNA or SAM). Non-replicating mRNA-based vaccines typically encode an antigen of interest and contain 5′ and 3′ untranslated regions (UTRs), a 5′ cap and a poly(A) tail; whereas self-amplifying RNAs also encode viral replication machinery that enables intracellular RNA amplification (Pardi, 2018).

There remains a need for an influenza vaccine that provides a broad and robust immune response against influenza virus. There particularly remains a need for an influenza vaccine that protects individuals from heterologous strains of influenza virus (i.e. a ‘universal vaccine’), including evolving seasonal and pandemic influenza virus strains of the future.

### SUMMARY OF THE INVENTION

It has been found that the immunogenicity of the influenza HA stem region is enhanced when delivered in the form of carrier-formulated mRNA. In particular, or in addition, it has been found the influenza HA stem polypeptides encoded by the carrier-formulated mRNAs induce a homologous, a heterologous and/or a heterosubtypic cross-reactive immunogenic responses.

The invention therefore provides a carrier-formulated mRNA comprising at least one coding sequence encoding an influenza HA stem polypeptide. As the mRNA encodes an influenza HA stem polypeptide, there is provided a carrier-formulated mRNA encoding the stem polypeptide but not an influenza HA head region. Therefore, the mRNA does not encode a full length influenza HA protein.

In some embodiments, the carrier is a lipid nanoparticle (LNP).

In some embodiments, the LNP comprises a PEG-modified lipid, optionally a non-cationic lipid, optionally a sterol, and a non-ionisable cationic lipid.

In some embodiments, the LNP comprises a PEG-modified lipid, optionally a non-cationic lipid, optionally a sterol, and an ionisable cationic lipid.

In some embodiments, the LNP comprises a cationic lipid having the following structure:

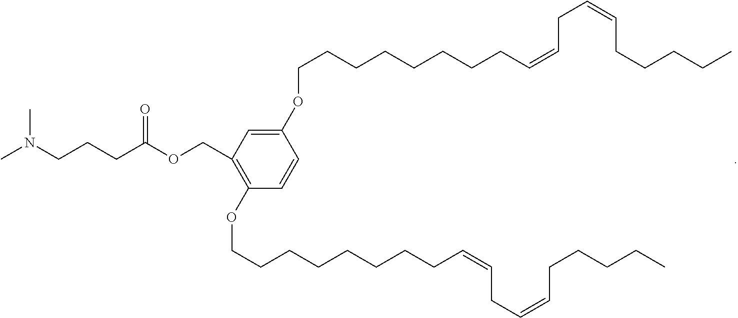 [SMILES: CCCCC/C=C\C/C=C\CCCCCCCCOc1ccc(OCCCCCCCC/C=C\C/C=C\CCCCC)c(COC(=O)CCCN(C)C)c1]

In some embodiments, the LNP comprises a PEG lipid selected from PEG-PE and PEG-DMG. In some embodiments, the LNP comprises a PEG lipid being PEG-DMG.

In some embodiments, the LNP comprises one or more neutral lipids and/or one or more steroid or steroid analogues.

In some embodiments, the non-cationic lipid is a neutral lipid, such as 1,2-distearoyl-sn-glycero-3-phosphocholine (DSPC), 1,2-dipalmitoyl-sn-glycero-3-phosphocholine (DPPC), 1-palmitoyl-2-oleoyl-sn-glycero-3-phosphocholine (POPC), 1,2-dioleoyl-sn-glycero-3-phosphoethanolamine (DOPE) or sphingomyelin (SM).

In some embodiments, the sterol is cholesterol. In some embodiments, the molar ratio of the cationic lipid to cholesterol is in the range from about 2:1 to about 1:1.

In some embodiments, the neutral lipid is 1,2-distearoyl-sn-glycero-3-phosphocholine (DSPC). In some embodiments, the molar ratio of the cationic lipid to DSPC is in the range from about 2:1 to about 8:1.

In some embodiments, the LNP comprise a PEG-modified lipid at around 0.5 to 15 molar %, a non-cationic lipid at around 5 to 25 molar %, a sterol at around 25 to 55 molar % and an ionisable cationic lipid at around 20 to 60 molar %.

In some embodiments, the LNP is composed of 40% w/w cationic lipid LKY750, 10% w/w zwitterionic lipid DSPC, 48% w/w cholesterol, and 2% w/w PEGylated lipid DMG.

In some embodiments, the LNP is 50 to 200 nm in diameter.

In some embodiments, the LNP have a polydispersity of 0.4 or less, such as 0.3 or less.

In some embodiments, the ratio of nucleotide (N) to phospholipid (P) is in the range of 1N:1P to 20N:1P, 1N:1P to 10N:1P, 2N:1P to 8N:1P, 2N:1P to 6N:1P or 3N:1P to 5N:1P.

In some embodiments, at least half of the mRNA is encapsulated in the LNP, suitably at least 85%, especially at least 95%, such as all of it.

In some embodiments, the carrier is a cationic nanoemulsion (CNE).

In some embodiments, the CNE is an oil-in-water emulsion of DOTAP and squalene stabilised with polysorbate 80 and/or sorbitan trioleate.

In some embodiments, the carrier is a lipidoid-coated iron oxide nanoparticle (LION).

In some embodiments, the mRNA comprises at least one additional coding sequence which encodes a protein nanoparticle.

In some embodiments, the protein nanoparticle is ferritin.

In some embodiments, the ferritin is selected from bacterial and insect ferritin.

In some embodiments, the ferritin is bacterial ferritin.

In some embodiments, the ferritin is H. pylori ferritin.

In some embodiments, the ferritin is insect ferritin.

In some embodiments, the insect ferritin comprises two monomeric subunits, such as a light chain and a heavy chain.

In some embodiments, each monomeric subunit may be linked to an influenza HA stem polypeptide derived from one or more subtypes.

In some embodiments, each monomeric subunit may be linked to an influenza HA stem polypeptide derived from one or more influenza A Group 1 subtypes and/or one or more influenza A Group 2 subtypes.

In some embodiments, the protein nanoparticle and the influenza HA stem polypeptide are directly or indirectly connected by a linker.

In some embodiments, the linker consists of 1 to 10 residues. In some embodiments, the linker consists of 2 to 5 residues.

In some embodiments, the linker comprises or consists of a polypeptide sequence GGSGG.

In some embodiments, the linker comprises or consists of a polypeptide sequence SGG.

In some embodiments, the influenza HA stem polypeptide is a polypeptide comprising or consisting of a full-length influenza HA stem region.

In some embodiments, the influenza HA stem polypeptide is a polypeptide comprising or consisting of an immunogenic fragment of an influenza HA stem region.

In some embodiments, the influenza HA stem polypeptide is a polypeptide comprising or consisting of an immunogenic variant of an influenza HA stem region.

In some embodiments, the influenza HA stem polypeptide comprises an amino acid sequence having at least 90%, 95%, 98% or 99% identity to the amino acid sequence set forth in any one of SEQ ID NO:1, SEQ ID NO: 2, SEQ ID NO: 3 or SEQ ID NO: 4.

In some embodiments, the influenza HA stem polypeptide comprises or consists of the amino acid sequence set forth in any one of SEQ ID NO: 1, SEQ ID NO: 2, SEQ ID NO: 3 or SEQ ID NO: 4.

In some embodiments, the influenza HA stem polypeptide is derived from influenza A, such as influenza A Group 1 or Group 2.

In some embodiments, the influenza HA stem polypeptide is derived from influenza A Group 1, such as influenza A subtype H1, H2, H5, H6, H8, H9, H11, H12, H13, H16, H17 or H18. In some embodiments, the influenza HA stem polypeptide is derived from influenza A H1.

In some embodiments, the influenza HA stem polypeptide comprises or consists of an amino acid sequence having at least 90%, 95%, 98% or 99% identity to the amino acid sequence set forth in any one of SEQ ID NO:1 or SEQ ID NO: 2.

In some embodiments, the influenza HA stem polypeptide comprises or consists of an amino acid sequence having at least 90%, 95%, 98% or 99% identity to the amino acid sequence set forth in any SEQ ID NO: 2.

In some embodiments, the influenza HA stem polypeptide comprises or consists of the amino acid sequence set forth in any one of SEQ ID NO:1 or SEQ ID NO: 2. In some embodiments, the influenza HA stem polypeptide comprises or consists of the amino acid sequence set forth in SEQ ID NO: 2.

In some embodiments, the influenza HA stem polypeptide is derived from influenza A Group 2, such as influenza A subtype H3, H4, H7, H10, H14 and H15. In some embodiments, the influenza HA stem polypeptide is derived from influenza A H3 or H10. In some embodiments, the influenza HA stem polypeptide is derived from influenza A H10.

In some embodiments, the influenza HA stem polypeptide comprises or consists of an amino acid sequence having at least 90%, 95%, 98% or 99% identity to the amino acid sequence set forth in any one of SEQ ID NO: 3 or SEQ ID NO: 4.

In some embodiments, the influenza HA stem polypeptide comprises or consists of the amino acid sequence set forth in any one of SEQ ID NO: 3 or SEQ ID NO: 4.

In some embodiments, the influenza HA stem polypeptide is 400 residues or fewer in length, especially 300 residues or fewer, in particular 250 residues or fewer, such as 220 residues or fewer.

In some embodiments, the influenza HA stem polypeptide is 130 residues or more in length, especially 160 residues or more, in particular 180 residues or more, such as 190 residues or more.

In some embodiments, the influenza HA stem polypeptide is 130 to 400 residues in length, especially 160 to 300, in particular 180 to 250, such as 190 to 220.

In some embodiments, the carrier-formulated mRNA comprises two or more coding sequences each encoding an influenza HA stem polypeptide, wherein the coding sequences are encoded on separate mRNA molecules.

In some embodiments, the carrier-formulated mRNA comprises two or more coding sequences each encoding an influenza HA stem polypeptide, wherein the coding sequences are encoded on the same mRNA molecule.

In some embodiments, the two or more coding sequences encode different influenza HA stem polypeptides.

In some embodiments, the two or more coding sequences comprise three or four coding sequences each encoding an influenza HA stem polypeptide.

In some embodiments, the two or more coding sequences encode influenza HA stem polypeptides derived from influenza A, such as influenza A Group 1 and/or influenza A Group 2.

In some embodiments, at least one of the two or more coding sequences encodes an influenza HA stem polypeptide derived from influenza A Group 1, such as influenza A subtype H1, H2, H5, H6, H8, H9, H11, H12, H13, H16, H17 and/or H18; and at least one of the two or more coding sequences encodes an influenza HA stem polypeptide derived from influenza A Group 2, such as influenza A subtype H3, H4, H7, H10, H14 and/or H15.

In some embodiments, at least one of the two or more coding sequences encodes an influenza HA stem polypeptide derived from influenza A H1; and at least one of the two or more coding sequences encodes an influenza HA stem polypeptide derived from influenza A H3 or H10.

In some embodiments, at least one of said two or more coding sequences encodes an influenza HA stem polypeptide derived influenza A subtype H1 and at least one of said two or more coding sequences encodes an influenza HA stem polypeptide derived from influenza A subtype H10.

In some embodiments, the influenza HA stem polypeptide derived from influenza A Group 1 comprises or consists of an amino acid sequence having at least 90%, 95%, 98% or 99% identity to the amino acid sequence set forth in any one of SEQ ID NO:1 or SEQ ID NO: 2. In some embodiments, the influenza HA stem polypeptide derived from influenza A Group 1 comprises or consists of an amino acid sequence having at least 90%, 95%, 98% or 99% identity to the amino acid sequence set forth in SEQ ID NO: 2.

In some embodiments, the influenza HA stem polypeptide derived from influenza A Group 1 comprises or consists of the amino acid sequence set forth in any one of SEQ ID NO:1 or SEQ ID NO: 2. In some embodiments, the influenza HA stem polypeptide derived from influenza A Group 1 comprises or consists of the amino acid sequence set forth in SEQ ID NO: 2.

In some embodiments, the influenza HA stem polypeptide derived from influenza A Group 2 comprises or consists of an amino acid sequence having at least 90%, 95%, 98% or 99% identity to the amino acid sequence set forth in any one of SEQ ID NO: 3 or SEQ ID NO: 4. In some embodiments, the influenza HA stem polypeptide derived from influenza A Group 2 comprises or consists of an amino acid sequence having at least 90%, 95%, 98% or 99% identity to the amino acid sequence set forth in SEQ ID NO: 4.

In some embodiments, the influenza HA stem polypeptide derived from influenza A Group 2 comprises or consists of the amino acid sequence set forth in any one of SEQ ID NO: 3 or SEQ ID NO: 4. In some embodiments, the influenza HA stem polypeptide derived from influenza A Group 2 comprises or consists of the amino acid sequence set forth in SEQ ID NO: 4.

In some embodiments, the mRNA comprises a 5′ cap, suitably m7G, cap0, cap1, cap2, a modified cap0 or a modified cap1 structure.

In some embodiments, the mRNA comprises a poly A tail and/or at least one poly(C) sequence. In some embodiments, the mRNA comprises a poly A tail comprising 30 to 200 adenosine nucleotides and/or at least one poly(C) sequence comprising 10 to 40 cytosine nucleotides.

In some embodiments, the mRNA comprises at least one 5′ untranslated region (UTR).

In some embodiments, the at least one heterologous 5′-UTR comprises or consists of a nucleic acid sequence derived from a 5′-UTR of a gene selected from HSD17B4, RPL32, ASAH1, ATP5A1, MP68, NDUFA4, NOSIP, RPL31, SLC7A3, TUBB4B and UBQLN2, or from a homolog, a fragment or variant of any one of these genes.

In some embodiments, the mRNA comprises at least one 3′ UTR.

In some embodiments, the at least one heterologous 3′-UTR comprises or consists of a nucleic acid sequence derived from a 3′-UTR of a gene selected from PSMB3, ALB7, alpha-globin (also referred to as “muag”), CASP1, COX6B1, GNAS, NDUFA1 and RPS9, or from a homolog, a fragment or a variant of any one of these genes.

In some embodiments, the mRNA comprises at least one chemical modification.

In some embodiments, the chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

In some embodiments, the mRNA is self-replicating.

In some embodiments, the self-replicating mRNA encodes (i) a RNA-dependent RNA polymerase which can transcribe RNA from the self-replicating RNA molecule and (ii) the influenza HA stem polypeptide.

In some embodiments, the mRNA comprises two open reading frames, the first of which encodes an alphavirus replicase and the second of which encodes the influenza HA stem polypeptide.

In some embodiments, the mRNA comprises three open reading frames, the first of which encodes an alphavirus replicase, the second of which encodes the influenza HA stem polypeptide and the third of which encodes a protein nanoparticle.

In some embodiments, the mRNA has the configuration 5′cap-5′UTR-non-structural proteins (NSP) 1-4-signal peptide-influenza HA stem polypeptide-linker-protein nanoparticle-3′UTR-polyA.

Also provided is an immunogenic composition comprising the carrier-formulated mRNA as defined herein, wherein the composition optionally comprises at least one pharmaceutically acceptable carrier.

In some embodiments, the composition is a multivalent composition comprising a plurality or at least one further mRNA in addition to the mRNA as defined herein.

In some embodiments, the multivalent composition comprises two or more mRNA as defined herein, such as two mRNA as defined herein. In some embodiments, the multivalent composition comprises two or more mRNA as defined herein, such as two mRNA as defined herein, each encoding a different influenza HA stem polypeptide.

In some embodiments, the two or more mRNA encode influenza HA stem polypeptides derived from influenza A, such as influenza A Group 1 and/or influenza A Group 2.

In some embodiments, at least one of the two or more mRNA encodes an influenza HA stem polypeptide derived from influenza A Group 1, such as influenza A subtype H1, H2, H5, H6, H8, H9, H11, H12, H13, H16, H17 and/or H18; and at least one of the two or more mRNA encodes an influenza HA stem polypeptide derived from influenza A Group 2, such as influenza A subtype H3, H4, H7, H10, H14 and/or H15.

In some embodiments, at least one of the two or more mRNA encodes an influenza HA stem polypeptide derived from influenza A H1; and at least one of the two or more mRNA encodes an influenza HA stem polypeptide derived from influenza A H3 or H10. In some embodiments, at least one of the two or more mRNA encodes an influenza HA stem polypeptide derived from influenza A H1; and at least one of the two or more mRNA encodes an influenza HA stem polypeptide derived from influenza A H10.

In some embodiments, at least one of the two or more mRNA are self-replicating. In some embodiments, each of the two or more mRNA are self-replicating.

Also provided is a vaccine comprising the carrier-formulated mRNA and/or the immunogenic composition as defined herein.

In some embodiments, the vaccine is a multivalent vaccine comprising a plurality or at least more than one of the mRNA, or a plurality or at least more than one of the composition as defined herein.

Also provided is a kit or kit of parts comprising the mRNA, and/or the composition, and/or the vaccine as defined herein, optionally comprising a liquid vehicle for solubilising, and, optionally, technical instructions providing information on administration and dosage of the components.

Also provided is the carrier-formulated mRNA, the immunogenic composition, the vaccine, the kit or kit of parts as defined herein for use as a medicament.

Also provided is the carrier-formulated mRNA, the composition, the vaccine, the kit or kit of parts as defined herein, for use in the treatment or prophylaxis of an infection with an influenza virus.

Also provided is the carrier-formulated mRNA, the composition, the vaccine, the kit or kit of parts as defined herein, for use in the treatment or prophylaxis of an infection with an influenza A virus.

In some embodiments, a single dose of the carrier-formulated mRNA is 0.01 to 1000 μg, especially 1 to 500 μg, in particular 10 to 250 μg of total mRNA.

In some embodiments, the use is for intramuscular administration.

In some embodiments, an immune response is elicited. In some embodiments, an adaptive immune response is elicited. In some embodiments, a protective adaptive immune response against an influenza virus is elicited. In some embodiments, a protective adaptive immune response against an influenza A virus is elicited.

In some embodiments, the elicited immune response reduces partially or completely the severity of one or more symptoms and/or time over which one or more symptoms of influenza virus infection are experienced by the subject.

In some embodiments, the elicited immune response reduces the likelihood of developing an established influenza virus infection after challenge.

In some embodiments, the elicited immune response slows progression of influenza.

Also provided is a method of treating or preventing a disorder, wherein the method comprises applying or administering to a subject in need thereof the carrier-formulated mRNA, the composition, the vaccine or the kit or kit of parts as defined herein.

In some embodiments, the disorder is an infection with an influenza virus. In some embodiments, the disorder is an infection with an influenza A virus.

In some embodiments, the subject in need is a mammalian subject, such as a human subject.

Also provided is a method of eliciting an immune response, wherein the method comprises applying or administering to a subject in need thereof the carrier-formulated mRNA, the composition, the vaccine or the kit or kit of parts as defined herein.

In some embodiments, the immune response is an adaptive immune response. In some embodiments, the immune response is a protective adaptive immune response against an influenza virus. In some embodiments, the immune response is a protective adaptive immune response against an influenza A virus.

In some embodiments, the immune response comprises a homologous, a heterologous and/or a heterosubtypic cross-reactive immunogenic responses against Influenza virus, such as against Influenza A virus, such as against Influenza A virus subtypes of Group 1 and/or Group 2.

In some embodiments, the subject in need is a mammalian subject, such as a human subject. Also provided is the use of carrier-formulated mRNA encoding an influenza HA stem polypeptide in the manufacture of a medicament.

Further embodiments of the invention are provided in the text below.

### BRIEF DESCRIPTION OF THE SEQUENCES

SEQ ID NO: 1: Polypeptide sequence of stabilised HA stem from A/New Caledonia/20/1999 (H1N1)

SEQ ID NO: 2: Polypeptide sequence of stabilised HA stem from A/Michigan/45/2015 (H1N1)

SEQ ID NO: 3: Polypeptide sequence of stabilised HA stem from A/Finland/486/2004 (H3N2)

SEQ ID NO: 4: Polypeptide sequence of stabilised HA stem from A/Jiangxi/IPB13/2013 (H10N8) (also referred to as “A/Jiangxi-Donghu/346/2013”)

SEQ ID NO: 5: Polypeptide sequence of H. pylori ferritin SEQ ID NO: 6: Polypeptide sequence of H1ssF_pylori (signal peptide-stabilised HA stem from A/New Caledonia/20/1999 (H1N1)-SGG-H. pylori ferritin)

SEQ ID NO: 7: Polypeptide sequence of H1ssF_pylori (signal peptide-stabilised HA stem from A/Michigan/45/2015 (H1N1)-SGG-H. pylori ferritin)

SEQ ID NO: 8: Polypeptide sequence of H3ssF_pylori (signal peptide-stabilised HA stem from A/Finland/486/2004 (H3N2)-SGG-H. pylori ferritin)

SEQ ID NO: 9: Polypeptide sequence of H10ssF_pylori (signal peptide-stabilised HA stem from A/Jiangxi/IPB13/2013 (H10N8)-SGG-H. pylori ferritin)

SEQ ID NO: 10: Polypeptide sequence of insect ferritin (iFH-F2A-iFL-6R; single polypeptide; insect ferritin heavy chain-self-cleaving construct-insect ferritin light chain)

SEQ ID NO: 11: Polypeptide sequence of H1ss_iH-F2A-H1ss_IL-6R (single polypeptide; signal peptide-stabilised HA stem from A/Michigan/45/2015 (H1N1)-GGSGG-insect ferritin heavy chain-self-cleaving construct-signal peptide-stabilised HA stem from A/Michigan/45/2015 (H1N1)-insect ferritin light chain)

SEQ ID NO: 12: Polypeptide sequence of H3ss_iH-F2A-H3ss_IL-6R (single polypeptide; signal peptide-stabilised HA stem from A/Finland/486/2004 (H3N2)-GGSGG-insect ferritin heavy chain-self-cleaving construct-signal peptide-stabilised HA stem from A/Finland/486/2004 (H3N2)-insect ferritin light chain)

SEQ ID NO: 13: Polypeptide sequence of H10ss_iH-F2A-H10ss_IL-6R (single polypeptide; signal peptide-stabilised HA stem from A/Jiangxi/IPB13/2013 (H10N8)-GGSGG-insect ferritin heavy chain-self-cleaving construct-signal peptide-stabilised HA stem from A/Jiangxi/IPB13/2013 (H10N8)-insect ferritin light chain)

SEQ ID NO: 14: Polypeptide sequence of H1ss_iH-F2A-H3ss_iL-6R (single polypeptide; signal peptide-stabilised HA stem from A/Michigan/45/2015 (H1N1)-GGSGG-insect ferritin heavy chain-self-cleaving construct-signal peptide-stabilised HA stem from A/Finland/486/2004 (H3N2)-insect ferritin light chain)

SEQ ID NO: 15: Polypeptide sequence of H1ss_iH-F2A-H10ss_iL-6R (single polypeptide; signal peptide-stabilised HA stem from A/Michigan/45/2015 (H1N1)-GGSGG-insect ferritin heavy chain-self-cleaving construct-signal peptide-stabilised HA stem from A/Jiangxi/IPB13/2013 (H10N8)-insect ferritin light chain)

SEQ ID NO: 16: Polypeptide sequence of H1NC99ss_iH-F2A-H10ss_iL-6R (single polypeptide; signal peptide-stabilised HA stem A/New Caledonia/20/1999 (H1N1)-GGSGG-insect ferritin heavy chain-self-cleaving construct-signal peptide-stabilised HA stem from A/Jiangxi/IPB13/2013 (H10N8)-insect ferritin light chain)

SEQ ID NO: 17: Organism: Trichoplusia ni SEQ ID NO: 18: Organism: Trichoplusia ni

SEQ ID NO: 19: Linker sequence

SEQ ID NO: 20: Nucleic acid sequence of H1ssF_pylori (from A/Michigan/45/2015 (H1N1))

SEQ ID NO: 21: Nucleic acid sequence of H10ssF_pylori (from A/Jiangxi/IPB13/2013 (H10N8))

SEQ ID NO: 22: Nucleic acid sequence of H1NC99ssF_pylori (from A/New Caledonia/20/1999 (H1N1))

SEQ ID NO: 23: Nucleic acid sequence of H1ss_iH-F2A-H10_iL-6R (from A/Michigan/45/2015 (H1N1) and A/Jiangxi/IPB13/2013 (H10N8), respectively)

SEQ ID NO: 24: Nucleic acid sequence of H1NC99ss_iH-F2A-H10_iL-6R ((from A/New Caledonia/20/1999 (H1N1) and A/Jiangxi/IPB13/2013 (H10N8), respectively)

### DESCRIPTION OF THE FIGURES

FIG. 1 depicts Study A: Anti-H1 stem IgG antibody titers by ELISA at 14 days post dose 2

FIG. 2 depicts Study B: Anti-H1 stem IgG antibody titers by ELISA at 14 days post dose 2

FIG. 3 depicts Study C: Anti-H1 stem IgG antibody titers by ELISA at 14 days post dose 2

FIG. 4 depicts Study A: Anti-H1/NC/99 IgG antibody titers by ELISA at 14 days post dose 2

FIGS. 5A and 5B depict Study B: Anti-H1/NC/99 IgG antibody titers by ELISA at 14 days post dose 2

FIGS. 6A and 6B depict Study A: Anti-H1/Mich/15 IgG antibody titers by ELISA at 14 days post dose 2

FIG. 7 depicts Study B: Anti-H1/Mich/15 IgG antibody titers by ELISA at 14 days post dose 2

FIG. 8 depicts Study C: Anti-H1/Mich/15 IgG antibody titers by ELISA at 14 days post dose 2

FIG. 9 depicts Study A: Anti-H2/Neth/99 IgG antibody titers by ELISA at 14 days post dose 2

FIG. 10 depicts Study B: Anti-H2/Neth/99 IgG antibody titers by ELISA at 14 days post dose 2

FIG. 11 depicts Study A: Anti-H9 IgG antibody titers by ELISA at 14 days post dose 2

FIG. 12 depicts Study B: Anti-H9 IgG antibody titers by ELISA at 14 days post dose 2

FIG. 13 depicts Study A: Anti-H18 IgG antibody titers by ELISA at 14 days post dose 2

FIG. 14 depicts Study B: Anti-H18 IgG antibody titers by ELISA at 14 days post dose 2

FIG. 15 depicts Study C: Anti-H2/Neth/99 IgG antibody titers by ELISA at 14 days post dose 2

FIG. 16 depicts Study C: Anti-H9 IgG antibody titers by ELISA at 14 days post dose 2

FIG. 17 depicts Study C: Anti-H18 IgG antibody titers by ELISA at 14 days post dose 2

FIG. 18 depicts Study B: Anti-H3 IgG antibody titers by ELISA at 14 days post dose 2

FIG. 19 depicts Study B: Anti-H7 IgG antibody titers by ELISA at 14 days post dose 2

FIGS. 20A and 20B depict Study B: Anti-H10 IgG antibody titers by ELISA at 14 days post dose 2

FIG. 21 depicts Study C: Anti-H3 IgG antibody titers by ELISA at 14 days post dose 2

FIG. 22 depicts Study C: Anti-H7 IgG antibody titers by ELISA at 14 days post dose 2

FIGS. 23A and 23B depict Study C: Anti-H10 IgG antibody titers by ELISA at 14 days post dose 2

FIG. 24 depicts Study A: Percentage of stem H1/Mich/2015 specific CD4+ T cell at 14 days post dose 2

FIG. 25 depicts Study B: Percentage of stem H1/Mich/2015 specific CD4+ T cell at 14 days post dose 2

FIG. 26 depicts Study C: Percentage of stem H1/Mich/2015 stem specific CD4+ T cell at 14 days post dose 2

FIG. 27 depicts Study A: Percentage of stem H1/Mich/2015 specific CD8+ T cell at 14 days post dose 2

FIG. 28 depicts Study B: Percentage of stem H1/Mich/2015 specific CD8+ T cell at 14 days post dose 2

FIG. 29 depicts Study C: Percentage of stem H1/Mich/2015 stem specific CD8+ T cell at 14 days post dose 2

FIG. 30 depicts Study B: Percentage of stem H10/Jiangxi-Donghu specific CD4+ T cell at 14 days post dose 2

FIG. 31 depicts Study B: Percentage of stem H10/Jiangxi-Donghu specific CD8+ T cell at 14 days post dose 2

FIG. 32 depicts Study C: Percentage of stem H10 A/Jiangxi/13/2013 stem specific CD4+ T cell at 14 days post dose 2

FIG. 33 depicts Study C: Percentage of stem H10 A/Jiangxi/13/2013 stem specific CD8+ T cell at 14 days post dose 2

FIG. 34 depicts microneutralization titers against H1/Mich/15, H1/NC/99 and H5A/n/04 at 14 days post dose 2

FIG. 35 depicts schematic of HA stem-H. pylori ferritin inserts

### DETAILED DESCRIPTION OF THE INVENTION

### Influenza HA Stem Polypeptide

Influenza hemagglutinin (HA) is the major surface antigen of the virion and the primary target of virus neutralizing antibodies. HA is a homotrimeric surface glycoprotein, with each monomer consisting of two disulfide-linked subunits (HA1, HA2), resulting from the proteolytic cleavage products of a single HA precursor protein. The HA1 chain forms a membrane-distal globular head and a part of the membrane-proximal stem (or ‘stalk’) region. The HA2 chain represents the major component of the stem region. The head of HA mediates receptor binding while the membrane-anchored stem is the main part of membrane fusion machinery. The invention disclosed herein relates to the influenza HA stem region when isolated from the influenza HA head region. The invention disclosed herein does not relate to the influenza HA stem region when comprised within the whole influenza HA polypeptide.

An ‘influenza HA stem polypeptide’ as used herein refers to a polypeptide comprising a full-length influenza HA stem region or an immunogenic fragment or variant of an influenza HA stem region. In one embodiment the influenza HA stem polypeptide is a polypeptide comprising or consisting of a full-length influenza HA stem region or an immunogenic fragment or variant of an influenza HA stem region.

In one embodiment the influenza HA stem polypeptide is desirably 400 residues or fewer in length, especially 300 residues or fewer, in particular 250 residues or fewer, such as 220 residues or fewer. In one embodiment the influenza HA stem polypeptide is desirably 130 residues or more in length, especially 160 residues or more, in particular 180 residues or more, such as 190 residues or more. In one embodiment the influenza HA stem polypeptide is desirably 130 to 400 residues in length, especially 160 to 300, in particular 180 to 250, such as 190 to 220.

In some embodiments, the influenza HA stem polypeptide comprises an amino acid sequence having at least 90%, 95%, 98% or 99% identity to the amino acid sequence set forth in any one of SEQ ID NO:1, SEQ ID NO: 2, SEQ ID NO: 3 or SEQ ID NO: 4.

In some embodiments, the influenza HA stem polypeptide comprises or consists of the amino acid sequence set forth in any one of SEQ ID NO: 1, SEQ ID NO: 2, SEQ ID NO: 3 or SEQ ID NO: 4.

Suitably the influenza HA stem polypeptide is derived from type A or B influenza virus. More suitably the influenza HA stem polypeptide is derived from type A influenza virus.

In one embodiment the influenza HA stem polypeptide is derived from influenza A, such as influenza A Group 1 or Group 2.

In some embodiments, the influenza HA stem polypeptide is derived from influenza A Group1 such as subtypes H1, H2, H5, H6, H8, H9, H11, H12, H13, H16, H17 or H18, more suitably H1 or H10, more suitably H1.

In some embodiments, the influenza HA stem polypeptide comprises or consists of an amino acid sequence having at least 90%, 95%, 98% or 99% identity to the amino acid sequence set forth in any one of SEQ ID NO:1 or SEQ ID NO: 2. In some embodiments, the influenza HA stem polypeptide comprises or consists of an amino acid sequence having at least 90%, 95%, 98% or 99% identity to the amino acid sequence set forth in SEQ ID NO: 2.

In some embodiments, the influenza HA stem polypeptide comprises or consists of the amino acid sequence set forth in any one of SEQ ID NO:1 or SEQ ID NO: 2. In some embodiments, the influenza HA stem polypeptide comprises or consists of the amino acid sequence set forth in SEQ ID NO: 2.

In some embodiments, the influenza HA stem polypeptide is derived from influenza A Group 2, such as subtypes H3, H4, H7, H10, H14 or H15. In some embodiments, the influenza HA stem polypeptide is derived from influenza A H3 or H10. In some embodiments, the influenza HA stem polypeptide is derived from influenza A H10.

In some embodiments, the influenza HA stem polypeptide comprises or consists of an amino acid sequence having at least 90%, 95%, 98% or 99% identity to the amino acid sequence set forth in any one of SEQ ID NO: 3 or SEQ ID NO: 4.

In some embodiments, the influenza HA stem polypeptide comprises or consists of the amino acid sequence set forth in any one of SEQ ID NO: 3 or SEQ ID NO: 4.

In an alternative embodiment the influenza HA stem polypeptide is derived from influenza B. In one embodiment the isolated influenza HA stem polypeptide is not derived from influenza A HA subtype H8, such as not derived from influenza A HA H9 Glade (H8, H9 and H12).

The influenza HA stem polypeptide is not a full-length influenza HA protein. The influenza HA stem polypeptide does not comprise an influenza HA head region, more suitably the influenza HA stem polypeptide does not comprise any additional regions from influenza HA.

The influenza HA stem polypeptide is also referred to herein as an ‘antigen’ or an ‘influenza stem polypeptide’ or ‘antigenic peptides or proteins’.

In some embodiments, the carrier-formulated mRNA comprises two or more coding sequences each encoding an influenza HA stem polypeptide, wherein the coding sequences are encoded on separate mRNA molecules

In some embodiments, the carrier-formulated mRNA comprises two or more coding sequences each encoding an influenza HA stem polypeptide, wherein the coding sequences are encoded on the same mRNA molecule.

In some embodiments, the two or more coding sequences encode different influenza HA stem polypeptides.

In some embodiments, the two or more coding sequences comprise three or four coding sequences each encoding an influenza HA stem polypeptide.

According to some embodiments, the two or more coding sequences encode influenza HA stem polypeptides derived from influenza A, such as influenza A Group 1 and/or influenza A Group 2.

In some embodiments, at least one of the two or more coding sequence encodes an influenza HA stem polypeptide derived from influenza A Group 1, such as influenza A subtype H1, H2, H5, H6, H8, H9, H11, H12, H13, H16, H17 and/or H18; and at least one of the two or more coding sequence encodes an influenza HA stem polypeptide derived from influenza A Group 2, such as influenza A subtype H3, H4, H7, H10, H14 and/or H15.

In some embodiments, at least one of the two or more coding sequence encodes an influenza HA stem polypeptide derived from influenza A H1; and at least one of the two or more coding sequence encodes an influenza HA stem polypeptide derived from influenza A H3 and/or H10.

In some embodiments, at least one of the two or more coding sequence encodes an influenza HA stem polypeptide derived from influenza A H1; and at least one of the two or more coding sequence encodes an influenza HA stem polypeptide derived from influenza A H10.

In some embodiments, the influenza HA stem polypeptide derived from influenza A Group 1 comprises or consists of an amino acid sequence having at least 90%, 95%, 98% or 99% identity to the amino acid sequence set forth in any one of SEQ ID NO:1 or SEQ ID NO: 2. In some embodiments, the influenza HA stem polypeptide derived from influenza A Group 1 comprises or consists of an amino acid sequence having at least 90%, 95%, 98% or 99% identity to the amino acid sequence set forth in SEQ ID NO: 2.

In some embodiments, the influenza HA stem polypeptide derived from influenza A Group 1 comprises or consists of the amino acid sequence set forth in any one of SEQ ID NO:1 or SEQ ID NO: 2. In some embodiments, the influenza HA stem polypeptide derived from influenza A Group 1 comprises or consists of the amino acid sequence set forth in SEQ ID NO: 2.

According to some embodiments, the influenza HA stem polypeptide derived from influenza A Group 2 comprises or consists of an amino acid sequence having at least 90%, 95%, 98% or 99% identity to the amino acid sequence set forth in any one of SEQ ID NO: 3 or SEQ ID NO: 4. According to some embodiments, the influenza HA stem polypeptide derived from influenza A Group 2 comprises or consists of an amino acid sequence having at least 90%, 95%, 98% or 99% identity to the amino acid sequence set forth in SEQ ID NO: 4.

In some embodiments, the influenza HA stem polypeptide derived from influenza A Group 2 comprises or consists of the amino acid sequence set forth in any one of SEQ ID NO: 3 or SEQ ID NO: 4. In some embodiments, the influenza HA stem polypeptide derived from influenza A Group 2 comprises or consists of the amino acid sequence set forth in SEQ ID NO: 4.

The influenza HA stem polypeptide may be comprised within a construct which comprises further polypeptide sequences. The further polypeptide sequences may include, for example, one or more signal peptides and/or one or more linkers and/or one or more protein nanoparticles. Accordingly, in some embodiments, the mRNA comprises at least one additional coding sequence which encodes one or more heterologous peptide or protein elements selected from a signal peptide, a linker and/or a protein nanoparticle.

In some embodiment, the mRNA comprises at least one additional coding sequence which encodes a protein nanoparticle. In some embodiments, the protein nanoparticle is ferritin. In some embodiments, the ferritin is selected from bacterial and insect ferritin.

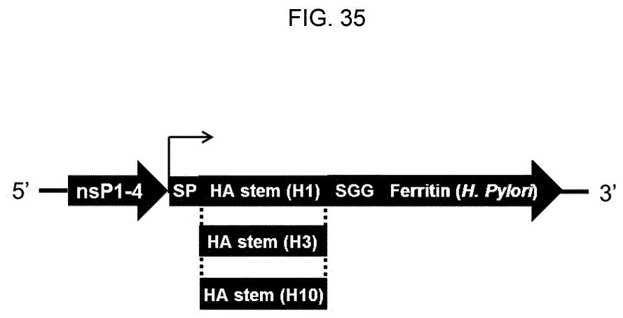

The influenza HA stem polypeptides used in some examples (e.g. Study A and Study B) are comprised within a construct which includes non-structural proteins 1-4 (nsP1-4), a signal peptide (SP), stabilised HA stem, a serine-glycine-glycine (SGG) linker, and H. pylori ferritin. The construct has the format: nsP1-4-SP-stabilised HA stem-SGG-H. pylori ferritin (FIG. 35).

The polypeptide sequences of the specific constructs used in these examples are SEQ ID NO: 7 (signal peptide-stabilised HA stem from A/Michigan/45/2015 (H1N1)-SGG-H. pylori ferritin), SEQ ID NO: 6 (signal peptide-stabilised HA stem from A/New Caledonia/20/1999 (H1N1)-SGG-H. pylori ferritin) and SEQ ID NO: 9 (signal peptide-stabilised HA stem from A/Jiangxi/IPB13/2013 (H10N8)-SGG-H. pylori ferritin). A further analogous construct which comprises an alternative HA stem polypeptide has the polypeptide sequence given in SEQ ID NO: 8 (signal peptide-stabilised HA stem from A/Finland/486/2004 (H3N2)-SGG-H. pylori ferritin).

Accordingly, in one embodiment, the influenza stem polypeptide is comprised within a construct having a polypeptide sequence having 80% or greater, such as 90% or greater, such as 95% or greater, such as 98% or greater, such as 99% or greater sequence identity to any one of SEQ ID NO: 6-9, more suitably SEQ ID NO: 7 or 9. Suitably the construct comprises or consists of any one of SEQ ID NOs: 6-9, more suitably SEQ ID NO: 7 or 9. In some embodiments, the influenza stem polypeptide is comprised within a construct having a polypeptide sequence having 80% or greater, such as 90% or greater, such as 95% or greater, such as 98% or greater, such as 99% or greater sequence identity to SEQ ID NO: 7. In some embodiments, the influenza stem polypeptide is comprised within a construct having a polypeptide sequence having 80% or greater, such as 90% or greater, such as 95% or greater, such as 98% or greater, such as 99% or greater sequence identity to SEQ ID NO: 9.

The influenza HA stem polypeptides used in some further examples (e.g. Study C) are comprised within a construct which comprises a signal peptide (SP), stabilised HA stem, a glycine-glycine-serine-glycine-glycine (GGSGG) linker, a self-cleaving construct and insect ferritin.

In some embodiments, insect ferritin comprises two monomeric subunits, such as a light chain and a heavy chain. According to some further embodiments, each monomeric subunit may be linked to an influenza HA stem polypeptide derived from one or more subtypes. In some embodiments, each monomeric subunit may be linked to an influenza HA stem polypeptide derived from one or more influenza A Group 1 subtypes and/or one or more influenza A Group 2 subtypes.

In some embodiments, each monomeric subunit may be linked to an influenza HA stem polypeptide derived from one influenza A subtype, e.g. from Group 1 or Group 2, to form an homodisplay. In some embodiments, the light chain and the heavy chain are both linked to an influenza HA stem polypeptide derived from the same subtype. In some embodiments, the light chain and the heavy chain are both linked to an influenza HA stem polypeptide derived from H1 or H3 or H10 subtype. In some embodiments, the light chain and the heavy chain are both linked to an influenza HA stem polypeptide derived from H1 or H10.

In some embodiments, the light chain and the heavy chain are both linked to an influenza HA stem polypeptide derived from the same influenza A strain, i.e. the influenza HA stem polypeptides linked to the light chain and the heavy chain are identical. In some embodiments, the light chain and the heavy chain are both linked to an influenza HA stem polypeptide derived from A/Michigan/45/2015 (H1N1), A/Finland/486/2004 (H3N2) or A/Jiangxi/IPB13/2013 (H10N8).

In some embodiments, the influenza HA stem polypeptide is comprised within a construct having a polypeptide sequence having 80% or greater, such as 90% or greater, such as 95% or greater, such as 98% or greater, such as 99% or greater sequence identity to any one of SEQ ID NO: 11-13, more suitably SEQ ID NO: 11 or 13. In one embodiment, the influenza HA stem polypeptide is comprised within a construct having a polypeptide sequence having 80% or greater, such as 90% or greater, such as 95% or greater, such as 98% or greater, such as 99% or greater sequence identity to SEQ ID NO: 11. In one embodiment, the influenza HA stem polypeptide is comprised within a construct having a polypeptide sequence having 80% or greater, such as 90% or greater, such as 95% or greater, such as 98% or greater, such as 99% or greater sequence identity to SEQ ID NO: 13.

In some embodiments, the construct comprises or consists of any one of SEQ ID NOs: 11-13, more suitably SEQ ID NO: 11 or 13. In some embodiments, the construct comprises or consists of SEQ ID NOs: 11. In some embodiments, the construct comprises or consists of SEQ ID NOs: 13.

Alternatively, each monomeric subunit may be linked to the influenza HA stem polypeptide derived from at least two different influenza A subtypes, e.g. from Group 1 and/or Group 2, to form an heterodisplay. In some embodiments, the light chain and the heavy chain are linked to influenza HA stem polypeptides derived from different influenza A subtypes. In some embodiments, the light chain is linked to an influenza HA stem polypeptide derived from influenza A Group 1 and the heavy chain is linked to an influenza HA stem polypeptide derived from influenza A Group 2, or inversely. In some embodiments, the light chain is linked to an influenza HA stem polypeptide derived from influenza A H1 subtype, and the heavy chain is linked to an influenza HA stem polypeptide derived from influenza A H3 or H10 subtype, or inversely. In some embodiments, the light chain is linked to an influenza HA stem polypeptide derived from influenza A H1 subtype, and the heavy chain is linked to an influenza HA stem polypeptide derived from influenza A H10 subtype, or inversely.

According to some embodiments, the heavy chain is linked to an influenza HA stem polypeptide derived from A/Michigan/45/2015 (H1N1) and the light chain is linked to an influenza HA stem polypeptide derived from A/Finland/486/2004 (H3N2) or A/Jiangxi/IPB13/2013 (H10N8). In some embodiments, the heavy chain is linked to an influenza HA stem polypeptide derived from A/Michigan/45/2015 (H1N1) and the light chain is linked to an influenza HA stem polypeptide derived from A/Jiangxi/IPB13/2013 (H10N8).

In some embodiments, the influenza HA stem polypeptide is comprised within a construct having a polypeptide sequence having 80% or greater, such as 90% or greater, such as 95% or greater, such as 98% or greater, such as 99% or greater sequence identity to any one of SEQ ID NO: 14-16, more suitably SEQ ID NO: 15. In some embodiments, the influenza HA stem polypeptide is comprised within a construct having a polypeptide sequence having 80% or greater, such as 90% or greater, such as 95% or greater, such as 98% or greater, such as 99% or greater sequence identity to SEQ ID NO: 15.

In some embodiments, the construct comprises or consists of any one of SEQ ID NOs: 14-16. In some embodiments, the construct comprises or consists of SEQ ID NO: 15.

Suitably the immune response elicited by the influenza HA stem polypeptide produces antibodies to influenza virus. More suitably, the elicited immune response produces anti-stem region antibodies.

A Type of influenza virus refers to influenza Type A, influenza Type B or influenza type C. The designation of a virus as a specific Type relates to sequence difference in the respective M1 (matrix) protein, M2 (ion channel) protein or NP (nucleoprotein). Type A influenza viruses are further divided into Group 1 and Group 2. These Groups are further divided into subtypes, which refers to classification of a virus based on the sequence of its HA protein. Examples of current commonly recognized subtypes are H1, H2, H3, H4, H5, H6, H7, H8, H9, H10, H11, H12, H13, H14, H15, H16, H17 or H18. Group 1 influenza subtypes are H1, H2, H5, H6, H8, H9, H11, H12, H13, H16, H17 and H18. Group 2 influenza subtypes are H3, H4, H7, H10, H14 and H15. Finally, the term strain refers to viruses within a subtype that differ from one another in that they have small, genetic variations in their genome.

In one embodiment the elicited immune response produces anti-Group 1 influenza A stem region antibodies, such as anti-H1, H2, H9 and/or H18 stem region antibodies. In some embodiments, the elicited immune response produces anti-Group 2 influenza A stem region antibodies, such as anti-H3, H7 and/or H10. In some embodiments, the elicited immune response produces anti-H7 and/or H10 stem region antibodies. Suitably the elicited immune response produces both anti-Group 1, such as anti-H1, H2, H9 and/or H18 stem region antibodies, and anti-Group 2, such as anti-H3, H7 and/or H10 influenza A stem region antibodies. In some embodiments, the elicited immune response produces both anti-Group 1, such as anti-H1, H2, H9 and/or H18 stem region antibodies, and anti-H7 and/or H10 influenza A stem region antibodies.

In some embodiments the elicited immune response produces one or more of anti-H1, H2, H3, H7, H9, H10 and/or H18 stem region antibodies. More suitably the elicited immune response produces one or more of anti-H1, H2, H7, H9, H10 and/or H18 stem region antibodies.

Suitably the elicited immune response produces all of anti-H1, H2, H3, H7, H9, H10 and/or H18 stem region antibodies. More suitably the elicited immune response produces all of anti-H1, H2, H7, H9, H10 and/or H18 stem region antibodies.

In some embodiments, the elicited immune response is homologous, heterologous and/or heterosubtypic cross-reactive.

The term “homologous” in the context of an elicited immune response will be recognized and understood by the person of ordinary skill in the art, and is e.g. an immune response which is elicited against the same strain, such as the same Influenza A strain. E.g. the carrier-formulated mRNA may comprise a coding sequence encoding a stem HA polypeptide derived from A/Michigan/45/2015 (H1N1) which may elicit an immune response against A/Michigan/45/2015 (H1N1) strain.

The term “heterologous” in the context of an elicited immune response will be recognized and understood by the person of ordinary skill in the art, and is e.g. an immune response which is elicited against different strains within a subtype, such as different Influenza A strains within a subtype such as H1 or H10 subtypes. E.g. the carrier-formulated mRNA may comprise a coding sequence encoding a stem HA polypeptide derived from A/Michigan/45/2015 (H1N1) which may elicit an immune response against A/New Caledonia/20/1999 (H1N1) strain.

The term “heterosubtypic” in the context of an elicited immune response will be recognized and understood by the person of ordinary skill in the art, and is e.g. an immune response which is elicited against different strains within one or more different subtypes, e.g. from Influenza A Group 1 and/or from Group 2 subtypes. E.g. the carrier-formulated mRNA may comprise a coding sequence encoding a stem HA polypeptide derived from A/Michigan/45/2015 (H1N1) which may elicit an immune response against A/Jiangxi/IPB13/2013 (H10N8).

### Full-Length Influenza HA Stem Region

In one embodiment the influenza HA stem polypeptide is a polypeptide comprising a full-length influenza HA stem region. Suitably the influenza HA stem polypeptide is a polypeptide consisting of a full-length influenza HA stem region.

The full-length influenza HA stem region is desirably 400 residues or fewer in length, especially 300 residues or fewer, in particular 250 residues or fewer, such as 220 residues or fewer. The full-length influenza HA stem region is desirably 130 residues or more in length, especially 160 residues or more, in particular 180 residues or more, such as 190 residues or more.

Suitably the full-length influenza HA stem region comprises or more suitably consists of a polypeptide sequence selected from SEQ ID NOs: 1-4. More suitably the full length influenza HA stem region comprises or more suitably consists of SEQ ID NO: 1, 2 or 4. More suitably the full length influenza HA stem region comprises or more suitably consists of SEQ ID NO: 2 or 4.

Further suitable full-length influenza HA stem regions are those disclosed in WO2013/044203, WO2015/183969 and in particular Table 2 of WO2018/045308.

### Immunogenic Fragments

In one embodiment the influenza HA stem polypeptide is a polypeptide comprising an immunogenic fragment of an influenza HA stem region. Suitably the influenza HA stem polypeptide is a polypeptide consisting of an immunogenic fragment of an influenza HA stem region.

In some embodiments, the immunogenic fragment of an influenza HA stem region of use in the present invention comprises, such as consists of, a fragment of a full length (such as native) influenza HA stem region which is capable of eliciting neutralising antibodies and/or a T cell response (such as a CD4 or CD8 T cell response) to influenza virus, such as to influenza A virus, suitably a protective immune response (e.g. reducing partially or completely the severity of one or more symptoms and/or time over which one or more symptoms are experienced by a subject following infection, reducing the likelihood of developing an established infection after challenge and/or slowing progression of illness (e.g. extending survival)).

Suitably the immunogenic fragment of an influenza HA stem region comprises one or more epitopes from a full-length influenza HA stem region, such as one, two or three or more epitopes.

The sequence of the immunogenic fragment of an influenza HA stem region may share 80% or greater, such as 90% or greater, such as 95% or greater, such as 98% or greater, such as 99% or greater, such as most suitably 100% identity with a corresponding sequence comprised within a full length influenza HA stem region, such as the sequences provided in SEQ ID NOs: 1-4, such as SEQ ID NO: 1, 2 or 4, most suitably SEQ ID NO: 2 or 4.

### Immunogenic Variants

In one embodiment the influenza HA stem polypeptide is a polypeptide comprising an immunogenic variant of an influenza HA stem region. Suitably the influenza HA stem polypeptide is a polypeptide consisting of an immunogenic variant of an influenza HA stem region.

In some embodiments, the immunogenic variant of an influenza HA stem region of use in the present invention comprises, such as consists of, a variant of a full length (such as native) influenza HA stem region which is capable of eliciting neutralising antibodies and/or a T cell response (such as a CD4 or CD8 T cell response) to influenza virus, such as to influenza A virus, suitably a protective immune response (e.g. reducing partially or completely the severity of one or more symptoms and/or time over which one or more symptoms are experienced by a subject following infection, reducing the likelihood of developing an established infection after challenge and/or slowing progression of illness (e.g. extending survival)).

The immunogenic variant of an influenza HA stem region may comprise, such as consist of, an amino acid sequence having at least 90%, such as at least 95%, such as at least 98%, such as at least 99%, such as 100% identity to the amino acid sequence set forth in SEQ ID NOs: 1-4, such as SEQ ID NO: 1, 2 or 4, most suitably SEQ ID NO: 2 or 4.

Suitably the immunogenic variant of an influenza HA stem region comprises one or more epitopes from a full-length influenza HA stem region, such as one, two or three or more epitopes.

### Sequence Alignments

Identity or homology with respect to a sequence is defined herein as the percentage of amino acid residues in the candidate sequence that are identical with the reference amino acid sequence after aligning the sequences and introducing gaps, if necessary, to achieve the maximum percent sequence identity, and not considering any conservative substitutions as part of the sequence identity.

Sequence identity can be determined by standard methods that are commonly used to compare the similarity in position of the amino acids of two polypeptides. Using a computer program such as BLAST or FASTA, two polypeptides are aligned for optimal matching of their respective amino acids (either along the full length of one or both sequences or along a pre-determined portion of one or both sequences). The programs provide a default opening penalty and a default gap penalty, and a scoring matrix such as PAM 250 (a standard scoring matrix; see Dayhoff, 1978) can be used in conjunction with the computer program. For example, the percent identity can then be calculated as: the total number of identical matches multiplied by 100 and then divided by the sum of the length of the longer sequence within the matched span and the number of gaps introduced into the shorter sequences in order to align the two sequences.

### Stability and Nanoparticles

For stable homotrimer assembly in its native environment, the influenza HA stem region requires the head region and the transmembrane domain. Arrangement in a homotrimer formation ensures antigenic conformational epitopes are presented. Accordingly, in one embodiment the influenza HA stem polypeptide is a stable influenza HA stem polypeptide, i.e. the polypeptide substantially retains its native conformation when expressed in a subject.

The influenza HA stem polypeptide may be synthetically stabilised (in the absence of head and transmembrane domains). Stabilisation may be achieved by helix stabilization, loop optimization, disulphide bond addition, and side-chain repacking (as disclosed in Corbett, 2019). Alternatively, or in addition, stabilisation may be achieved by providing the stem region in the form of a multimer, such as a homotrimer or a heterotrimer.

The influenza HA stem polypeptide may be provided ‘naked’ within the carrier-formulated mRNA, i.e. not bound to other stabilizing proteins or components. Alternatively, the influenza HA stem polypeptide may be co-expressed in the host with one or more other stabilizing proteins. In a particular embodiment, the influenza HA stem polypeptide is presented on the surface of nanoparticles, such as protein nanoparticles, such as those disclosed in Diaz et al 2018 including ferritin, lumazine and encapsulin.

When provided in the form of a homotrimer or a heterotrimer, the influenza HA stem polypeptide is most suitably displayed on self-assembling protein nanoparticles, such as most suitably ferritin nanoparticles, such as more suitably insect or bacterial ferritin nanoparticles.

According to some embodiments, the protein nanoparticles are bacterial ferritin nanoparticles. According to some embodiments, the protein nanoparticles are H. pylori ferritin nanoparticles (such as those disclosed in Corbett, 2019, WO2013/044203, WO2015/183969 and WO2018/045308). When co-expressed in the host, a H. pylori ferritin linked to an influenza HA stem polypeptide will self-assembles with other H. pylori ferritins each linked to influenza HA stem polypeptides to form a nanoparticle displaying a plurality of influenza HA stem polypeptides allowing their assembly in one or more homotrimers and/or one or more heterotrimers.

In some embodiments, the protein nanoparticles are insect ferritin nanoparticles. In some embodiments, the protein nanoparticles are Trichoplusia ni ferritin nanoparticles.

In some embodiments, insect ferritin comprises two monomeric subunits, such as a light chain and a heavy chain. In some embodiments the light chain is the light chain of Trichoplusia ni ferritin and the heavy chain is the heavy chain of Trichoplusia ni ferritin (SEQ ID NO: 17 and 18).

According to some further embodiments, each monomeric subunit may be linked to an influenza HA stem polypeptide derived from one or more subtypes. In some embodiments, each monomeric subunit may be linked to an influenza HA stem polypeptide derived from one or more influenza A Group 1 subtypes and/or one or more influenza A Group 2 subtypes. When co-expressed in the host, each monomeric subunit of insect ferritin which is linked to an influenza HA stem polypeptide, e.g. each monomeric may be linked to an influenza HA stem polypeptide that is different or not, will self-assemble with other monomeric subunits of insect ferritin, each monomeric subunit being linked to an influenza HA stem polypeptide, to form a nanoparticle displaying a plurality of influenza HA stem polypeptides allowing their assembly in one or more homotrimers and/or one or more heterotrimers.

Suitably the ferritin, such as the bacterial ferritin, such as the H. pylori ferritin, and the influenza HA stem polypeptide are directly or indirectly connected by a linker, e.g. the linker consists of 1 to 10 residues, e.g. of 2 to 5 residues, such as a linker comprising the polypeptide sequence SGG, such as consisting of the polypeptide sequence SGG.

In some embodiments, the ferritin, such the insect ferritin, and the influenza HA stem polypeptide are directly or indirectly connected by a linker, e.g. the linker consists of 1 to 10 residues, such as a linker comprising the polypeptide sequence GGSGG, such as consisting of the polypeptide sequence GGSGG (SEQ ID NO: 19).

In some embodiments, the two monomeric subunits of insect ferritin, such as the light chain and the heavy chain, are directly or indirectly connected by a self-cleaving construct. In some embodiments, the self-cleaving construct is a furin self-cleaving construct, e.g. the self-cleaving construct is F2A. In some embodiments, the self-cleaving construct is positioned after the linker when considering the sense 5′ to 3′.

### Additional Antigens

The present invention may involve a plurality of antigenic components, for example with the objective to elicit a broad immune response to influenza virus. Consequently, more than one antigen may be present, more than one polynucleotide encoding an antigen may be present, one polynucleotide encoding more than one antigen may be present or a mixture of antigen(s) and polynucleotide(s) encoding antigen(s) may be present. Polysaccharides such as polysaccharide conjugates may also be present.

In some embodiments, by the term antigen is meant a peptide, a protein or a polypeptide which is capable of eliciting an immune response. Suitably the antigen comprises at least one B or T cell epitope. The elicited immune response may be an antigen specific B cell response, which produces neutralizing antibodies. The elicited immune response may be an antigen specific T cell response, which may be a systemic and/or a local response. The antigen specific T cell response may comprise a CD4+ T cell response, such as a response involving CD4+ T cells expressing a plurality of cytokines, e.g. IFNgamma, TNFalpha and/or IL2. Alternatively, or additionally, the antigen specific T cell response comprises a CD8+ T cell response, such as a response involving CD8+ T cells expressing a plurality of cytokines, e.g., IFNgamma, TNFalpha and/or IL2.

mRNA

Messenger RNA (mRNA) can direct the cellular machinery of a subject to produce proteins. mRNA may be circular or branched, but will generally be linear. The mRNA may be circular or linear.

mRNA used herein are preferably provided in purified or substantially purified form i.e. substantially free from proteins (e.g., enzymes), other nucleic acids (e.g. DNA and nucleoside phosphate monomers), and the like, generally being at least about 50% pure (by weight), and usually at least 90% pure, such as at least 95% or at least 98% pure.

mRNA may be prepared in many ways e.g. by chemical synthesis in whole or in part, by digesting longer nucleic acids using nucleases (e.g. restriction enzymes), by joining shorter nucleic acids or nucleotides (e.g. using ligases or polymerases), from genomic or cDNA libraries, etc. In particular, mRNA may be prepared enzymatically using a DNA template.

The term mRNA as used herein includes conventional mRNA or mRNA analogs, such as those containing modified backbones or modified bases (e.g. pseudouridine, or the like). mRNA, may or may not have a 5′ cap.

In some embodiments, the mRNA comprises a sequence which encodes at least one antigen. Typically, the nucleic acids of the invention will be in recombinant form, i.e. a form which does not occur in nature. For example, the mRNA may comprise one or more heterologous nucleic acid sequences (e.g. a sequence encoding another antigen and/or a control sequence such as a promoter or an internal ribosome entry site) in addition to the sequence encoding the antigen.

In some embodiments, the mRNA is an artificial mRNA.

The term “artificial mRNA” as used herein is intended to refer to an mRNA that does not occur naturally. In other words, an artificial mRNA may be understood as a non-natural mRNA molecule. Such mRNA molecules may be non-natural due to its individual sequence (e.g. G/C content modified coding sequence, UTRs) and/or due to other modifications, e.g. structural modifications of nucleotides. Typically, artificial mRNA may be designed and/or generated by genetic engineering to correspond to a desired artificial sequence of nucleotides. In this context, an artificial mRNA is a sequence that may not occur naturally, i.e. a sequence that differs from the wild type sequence/the naturally occurring sequence by at least one nucleotide. The term “artificial mRNA” is not restricted to mean “one single mRNA molecule” but is understood to comprise an ensemble of essentially identical mRNA molecules. Accordingly, it may relate to a plurality of essentially identical mRNA molecules.

Alternatively, or in addition, the sequence or chemical structure of the nucleic acid may be modified compared to a naturally occurring sequence which encodes the antigen. The sequence of the nucleic acid molecule may be modified, e.g. to increase the efficacy of expression or replication of the nucleic acid, or to provide additional stability or resistance to degradation.

In some embodiments, the carrier-formulated mRNA is a modified and/or stabilized nucleic acid, such as a modified and/or stabilized artificial nucleic acid.

According to some embodiments, the mRNA may thus be provided as a “stabilized artificial nucleic acid” or “stabilized coding nucleic acid” that is to say a nucleic acid showing improved resistance to in vivo degradation and/or a nucleic acid showing improved stability in vivo, and/or a nucleic acid showing improved translatability in vivo. In the following, specific suitable modifications/adaptations in this context are described which are suitably to “stabilize” the nucleic acid. In the following, suitable modifications are described that are capable of “stabilizing” the mRNA.

mRNA may also be codon optimised. In some embodiments, the mRNA comprises at least one codon modified coding sequence. In some embodiments, the at least one coding sequence of the mRNA is a codon modified coding sequence. Suitably, the amino acid sequence encoded by the at least one codon modified coding sequence is not being modified compared to the amino acid sequence encoded by the corresponding wild type or reference coding sequence.

The term “codon modified coding sequence” relates to coding sequences that differ in at least one codon (triplets of nucleotides coding for one amino acid) compared to the corresponding wild type or reference coding sequence.

In some embodiments, mRNA may be codon optimised for expression in human cells. By “codon optimised” is intended modification with respect to codon usage may increase translation efficacy and/or half-life of the nucleic acid.

A poly A tail (e.g., of about 30 adenosine residues or more) may be attached to the 3′ end of the RNA to increase its half-life. Accordingly, in some embodiments, the mRNA comprises at least one poly(A) sequence.

The terms “poly(A) sequence”, “poly(A) tail” or “3′-poly(A) tail” as used herein will be recognized and understood by the person of ordinary skill in the art, and are e.g. intended to be a sequence of adenosine nucleotides, typically located at the 3′-end of a linear RNA (or in a circular RNA), of up to about 1000 adenosine nucleotides. In some embodiments, the poly(A) sequence is essentially homopolymeric, e.g. a poly(A) sequence of e.g. 100 adenosine nucleotides has essentially the length of 100 nucleotides. In other embodiments, the poly(A) sequence is interrupted by at least one nucleotide different from an adenosine nucleotide, e.g. a poly(A) sequence of e.g. 100 adenosine nucleotides may have a length of more than 100 nucleotides (comprising 100 adenosine nucleotides and in addition said at least one nucleotide—or a stretch of nucleotides—different from an adenosine nucleotide).

The poly(A) sequence may comprise about 10 to about 500 adenosine nucleotides, about 10 to about 200 adenosine nucleotides, about 40 to about 200 adenosine nucleotides, or about 40 to about 150 adenosine nucleotides. Suitably, the length of the poly(A) sequence may be at least about or even more than about 10, 50, 64, 75, 100, 200, 300, 400, or 500 adenosine nucleotides. In certain embodiments the mRNA comprises at least one poly(A) sequence comprising 30 to 200 adenosine nucleotides, wherein the 3′ terminal nucleotide of said mRNA is an adenosine.

In some embodiments, the mRNA comprises at least one poly(A) sequence comprising about 30 to about 200 adenosine nucleotides. In some embodiments, the poly(A) sequence comprises about 64 adenosine nucleotides (A64). In some embodiments, the poly(A) sequence comprises about 100 adenosine nucleotides (A100). In other embodiments, the poly(A) sequence comprises about 150 adenosine nucleotides.

In further embodiments, the mRNA comprises at least one poly(A) sequence comprising about 100 adenosine nucleotides, wherein the poly(A) sequence is interrupted by non-adenosine nucleotides, suitably by 10 non-adenosine nucleotides (A30-N10-A70).

The poly(A) sequence as defined herein may be located at the 3′ terminus of the mRNA. In some embodiments, the poly(A) sequence as defined herein may be located directly at the 3′ terminus of the mRNA.

In some embodiments, the 3′-terminal nucleotide (that is the last 3′-terminal nucleotide in the polynucleotide chain) is the 3′-terminal A nucleotide of the at least one poly(A) sequence. The term “directly located at the 3′ terminus” has to be understood as being located exactly at the 3′ terminus—in other words, the 3′ terminus of the nucleic acid consists of a poly(A) sequence terminating with an A nucleotide.

In an embodiment, the mRNA sequence comprises a poly(A) sequence of at least 70 adenosine nucleotides, wherein the 3′-terminal nucleotide is an adenosine nucleotide.

In some embodiments, the poly(A) sequence of the RNA is obtained from a DNA template during RNA in vitro transcription. In other embodiments, the poly(A) sequence is obtained in vitro by common methods of chemical synthesis without being necessarily transcribed from a DNA template. In other embodiments, poly(A) sequences are generated by enzymatic polyadenylation of the RNA (after RNA in vitro transcription) using commercially available polyadenylation kits and corresponding protocols known in the art, or alternatively, by using immobilized poly(A)polymerases e.g. using a methods and means as described in WO2016/174271, the entire contents of which are hereby incorporated by reference.

In some embodiments, the RNA comprises a poly(A) sequence obtained by enzymatic polyadenylation, wherein the majority of RNA molecules comprise about 100 (+/−20) to about 500 (+/−50). In some embodiments, the RNA comprises a poly(A) sequence obtained by enzymatic polyadenylation, wherein the majority of RNA molecules comprise about 250 (+/−20) adenosine nucleotides.

In other embodiments, the RNA comprises a poly(A) sequence derived from a template DNA and at least one additional poly(A) sequence generated by enzymatic polyadenylation, e.g. as described in WO2016/091391, the entire contents of which are hereby incorporated by reference.

In further embodiments, the RNA comprises at least one poly(C) sequence.

The term “poly(C) sequence” as used herein is intended to be a sequence of cytosine nucleotides of up to about 200 cytosine nucleotides. In some embodiments, the poly(C) sequence comprises about 10 to about 200 cytosine nucleotides, about 10 to about 100 cytosine nucleotides, about 20 to about 70 cytosine nucleotides, about 20 to about 60 cytosine nucleotides, or about 10 to about 40 cytosine nucleotides. In an embodiment, the poly(C) sequence comprises about 30 cytosine nucleotides.

In some embodiments, the mRNA comprises a poly A tail, such as comprising 30 to 200 adenosine nucleotides and/or at least one poly(C) sequence, such as comprising 10 to 40 cytosine nucleotides.

The 5′ end of the RNA may be capped, for example with a modified ribonucleotide with the structure m7G (5′) ppp (5′) N (cap 0 structure) or a derivative thereof, which can be incorporated during RNA synthesis or can be enzymatically engineered after RNA transcription (e.g., by using Vaccinia Virus Capping Enzyme (VCE) consisting of mRNA triphosphatase, guanylyl-transferase and guanine-7-methytransferase, which catalyzes the construction of N7-monomethylated cap 0 structures). Cap 0 structure plays an important role in maintaining the stability and translational efficacy of the RNA molecule. The 5′ cap of the mRNA molecule may be further modified by a 2′-O-Methyltransferase which results in the generation of a cap 1 structure (m7Gppp [m2′-O] N), which may further increase translation efficacy. In some embodiments, the mRNA comprises a 5′ cap. In some embodiments, the 5′ cap is m7G, cap0, cap1, cap2, a modified cap0 or a modified cap1 structure.

The term “5′-cap structure” as used herein will be recognized and understood by the person of ordinary skill in the art, and is e.g. intended to refer to a 5′ modified nucleotide, particularly a guanine nucleotide, positioned at the 5′-end of an RNA, e.g. an mRNA. In some embodiments, the 5′-cap structure is connected via a 5′-5′-triphosphate linkage to the mRNA.

5′-cap structures which may be suitable are cap0 (methylation of the first nucleobase, e.g. m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse cap analogue), modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

A 5′-cap (cap0 or cap1) structure may be formed in chemical RNA synthesis or in RNA in vitro transcription (co-transcriptional capping) using cap analogues.

In some embodiments, the mRNA comprises at least one 5′ untranslated region (UTR) and/or at least one 3′ UTR.

In some embodiments, the at least one heterologous 5′-UTR comprises or consists of a nucleic acid sequence derived from a 5′-UTR of a gene selected from HSD17B4, RPL32, ASAH1, ATP5A1, MP68, NDUFA4, NOSIP, RPL31, SLC7A3, TUBB4B and UBQLN2, or from a homolog, a fragment or variant of any one of these genes.

In some embodiments, the at least one heterologous 3′-UTR comprises or consists of a nucleic acid sequence derived from a 3′-UTR of a gene selected from PSMB3, ALB7, CASP1, COX6B1, GNAS, NDUFA1 and RPS9, or from a homolog, a fragment or a variant of any one of these genes.

mRNA may comprise one or more nucleotide analogs or modified nucleotides. As used herein, “nucleotide analog” or “modified nucleotide” refers to a nucleotide that contains one or more chemical modifications (e.g., substitutions) in or on the nitrogenous base of the nucleoside (e.g. cytosine (C), thymine (T) or uracil (U)), adenine (A) or guanine (G)). A nucleotide analog can contain further chemical modifications in or on the sugar moiety of the nucleoside (e.g. ribose, modified ribose, six-membered sugar analog, or open-chain sugar analog), or the phosphate. The preparation of nucleotides and modified nucleotides and nucleosides are well-known in the art, see the following references: U.S. Pat. Nos. 4,373,071, 4,458,066, 4,500,707, 4,668,777, 4,973,679, 5,047,524, 5,132,418, 5,153,319, 5,262,530, 5,700,642. Many modified nucleosides and modified nucleotides are commercially available.

Modified nucleobases (chemical modifications) which can be incorporated into modified nucleosides and nucleotides and be present in the mRNA molecules include: m5C (5-methylcytidine), m5U (5-methyluridine), m6A (N6-methyladenosine), s2U (2-thiouridine), Um (2′-O-methyluridine), m1A (1-methyladenosine); m2A (2-methyladenosine); Am (2-1-O-methyladenosine); ms2m6A (2-methylthio-N6-methyladenosine); i6A (N6-isopentenyladenosine); ms2i6A (2-methylthio-N6isopentenyladenosine); io6A (N6-(cis-hydroxyisopentenyl)adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A (N6-hydroxynorvalylcarbamoyl adenosine); ms2hn6A (2-methylthio-N6-hydroxynorvalyl carbamoyladenosine); Ar(p) (2′-O-ribosyladenosine (phosphate)); I (inosine); mil (1-methylinosine); m'Im (I,2′-O-dimethylinosine); m3C (3-methylcytidine); Cm (2′-O-methylcytidine); s2C (2-thiocytidine); ac4C (N4-acetylcytidine); f5C (5-fonnylcytidine); m5Cm (5,2-O-di methylcytidine); ac4Cm (N4-acetyl-2-O-methylcytidine); k2C (lysidine); m1G (1-methylguanosine); m2G (N2-methylguanosine); m7G (7-methylguanosine); Gm (2′-O-methylguanosine); m22G (N2,N2-dimethylguanosine); m2Gm (N2,2′-O-dimethylguanosine); m22Gm (N2,N2,2′-O-trimethylguanosine); Gr(p) (2′-O-ribosylguanosine (phosphate)); yW (wybutosine); o2yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-O-dimethyluridine); s4U (4-thiouridine); m5s2U (5-methyl-2-thiouridine); s2Um (2-thio-2′-O-methyluridine); acp3U (3-(3-amino-3-carboxypropyl)uridine); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl)uridine)); mchm5U (5-(carboxyhydroxymethyl)uridine methyl ester); mcm5U (5-methoxycarbonyl methyluridine); mcm5Um (S-methoxycarbonylmethyl-2-O-methyluridine); mcm5s2U (5-methoxycarbonylmethyl-2-thiouridine); nm5s2U (5-aminomethyl-2-thiouridine); mnm5U (5-methylaminomethyluridine); mnm5s2U (5-methylaminomethyl-2-thiouridine); mnm5se2U (5-methylaminomethyl-2-selenouridine); ncm5U (5-carbamoylmethyl uridine); ncm5Um (5-carbamoylmethyl-2′-O-methyluridine); cmnm5U (5-carboxymethylaminomethyluridine); cnmm5Um (5-carboxymethy 1 aminomethyl-2-L-O-methyl uridine); cmnm5s2U (5-carboxymethylaminomethyl-2-thiouridine); m62A (N6,N6-dimethyladenosine); Tm (2′-O-methylinosine); m4C (N4-methylcytidine); m4Cm (N4,2-O-dimethylcytidine); hm5C (5-hydroxymethylcytidine); m3U (3-methyluridine); cm5U (5-carboxymethyluridine); m6Am (N6,2′-O-dimethyladenosine); rn62Am (N6,N6,0-2-trimethyladenosine); m2′7G (N2,7-dimethylguanosine); m2′2′7G (N2,N2,7-trimethylguanosine); m3Um (3,2′-O-dimethyluridine); m5D (5-methyldihydrouridine); f5Cm (5-formyl-2′-O-methylcytidine); mlGm (I,2′-0-dimethylguanosine); m′Am (1,2-O-dimethyl adenosine) irinomethyluridine); tm5s2U (S-taurinomethyl-2-thiouridine)); iniG-14 (4-demethyl guanosine); imG2 (isoguanosine); ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-methyluracil, 5-(02-06)-alkenyluracil, 5-(02-06)-alkynyluracil, 5-(hydroxymethyl)uracil, 5-chlorouracil, 5-fluorouracil, 5-bromouracil, 5-hydroxycytosine, 5-(C₁-C₆)-alkylcytosine, 5-methylcytosine, 5-(C₂-C₆)-alkenylcytosine, 5-(C₂-C₆)-alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C₂-C₆)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), m5C, m5U, m6A, s2U, W, or 2′-O-methyl-U. Many of these modified nucleobases and their corresponding ribonucleosides are available from commercial suppliers.

According to some embodiments, the mRNA comprises at least one chemical modification.

In some embodiments, the chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In one embodiment, the chemical modification is N1-methylpseudouridine.

The mRNA may encode more than one antigen. For example, the mRNA encoding an antigen protein may encode only the antigen or may encode additional proteins.

According to some embodiment, the mRNA may be non-replicating.

According to some embodiments, the mRNA is replicating, also known as self-amplifying. A self-amplifying mRNA (SAM) molecule may be an alphavirus-derived mRNA replicon. mRNA amplification can also be achieved by the provision of a non-replicating mRNA encoding an antigen in conjunction with a separate mRNA encoding replication machinery.

Self-replicating RNA molecules are well known in the art and can be produced by using replication elements derived from, e.g., alphaviruses, and substituting the structural viral proteins with a nucleotide sequence encoding a protein of interest. A self-replicating RNA molecule is typically a +-strand molecule which can be directly translated after delivery to a cell, and this translation provides a RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded antigen, or may be transcribed to provide further transcripts with the same sense as the delivered RNA which are translated to provide in situ expression of the antigen. The overall result of this sequence of transcriptions is a huge amplification in the number of the introduced replicon RNAs and so the encoded antigen becomes a major polypeptide product of the cells.

Suitable alphavirus replicons can use a replicase from a Sindbis virus, a Semliki forest virus, an eastern equine encephalitis virus, a Venezuelan equine encephalitis virus, etc. Mutant or wild-type virus sequences can be used e.g. the attenuated TC83 mutant of VEEV has been used in replicons, see the following reference: WO2005/113782.

In certain embodiments, the self-replicating RNA molecule described herein encodes (i) a RNA-dependent RNA polymerase which can transcribe RNA from the self-replicating RNA molecule and (ii) an antigen, e.g. the influenza HA stem polypeptide. The polymerase can be an alphavirus replicase e.g. comprising one or more of alphavirus proteins nsPl, nsP2, nsP3 and nsP4 (wherein nsP stands for non-structural protein).

Whereas natural alphavirus genomes encode structural virion proteins in addition to the non-structural replicase polyprotein, the self-replicating RNA molecules do not encode alphavirus structural proteins. Thus, the self-replicating RNA can lead to the production of genomic RNA copies of itself in a cell, but not to the production of RNA-containing virions. The inability to produce these virions means that, unlike a wild-type alphavirus, the self-replicating RNA molecule cannot perpetuate itself in infectious form. The alphavirus structural proteins which are necessary for perpetuation in wild-type viruses are absent from self-replicating RNAs of the present disclosure and their place is taken by gene(s) encoding the immunogen of interest, such that the subgenomic transcript encodes the immunogen rather than the structural alphavirus virion proteins.

Thus, a self-replicating RNA molecule useful with the invention may have two open reading frames. The first (5′) open reading frame encodes a replicase, such as an alphavirus replicase; the second (3′) open reading frame encodes an antigen, e.g. the influenza HA stem polypeptide. In some embodiments the RNA may have additional (e.g. downstream) open reading frames e.g. to encode further antigens or to encode accessory polypeptides. In some embodiments, the RNA molecule comprises three open reading frames, the first of which encodes an alphavirus replicase, the second of which encodes the influenza HA stem polypeptide and the third of which encodes a protein nanoparticle.

In certain embodiments, the self-replicating RNA molecule disclosed herein has a 5′ cap (e.g. a 7-methylguanosine). This cap can enhance in vivo translation of the RNA. In some embodiments the 5′ sequence of the self-replicating RNA molecule must be selected to ensure compatibility with the encoded replicase.

A self-replicating RNA molecule may have a 3′ poly-A tail. It may also include a poly-A polymerase recognition sequence (e.g. AAUAAA) near its 3′ end.

Self-replicating RNA molecules can have various lengths, but they are typically 5000-25000 nucleotides long. Self-replicating RNA molecules will typically be single-stranded. Single-stranded RNAs can generally initiate an adjuvant effect by binding to TLR7, TLR8, RNA helicases and/or PKR. RNA delivered in double-stranded form (dsRNA) can bind to TLR3, and this receptor can also be triggered by dsRNA which is formed either during replication of a single-stranded RNA or within the secondary structure of a single-stranded RNA.

In another embodiment, a self-replicating RNA may comprise two separate RNA molecules, each comprising a nucleotide sequence derived from an alphavirus: one RNA molecule comprises a RNA construct for expressing alphavirus replicase, and one RNA molecule comprises a RNA replicon that can be replicated by the replicase in trans. The RNA construct for expressing alphavirus replicase comprises a 5′-cap. See WO2017/162265.

The self-replicating RNA can conveniently be prepared by in vitro transcription (IVT). IVT can use a (cDNA) template created and propagated in plasmid form in bacteria, or created synthetically (for example by gene synthesis and/or polymerase chain-reaction (PCR) engineering methods). For instance, a DNA-dependent RNA polymerase (such as the bacteriophage T7, T3 or SP6 RNA polymerases) can be used to transcribe the self-replicating RNA from a DNA template. Appropriate capping and poly-A addition reactions can be used as required (although the replicon's poly-A is usually encoded within the DNA template). These RNA polymerases can have stringent requirements for the transcribed 5′ nucleotide(s) and in some embodiments these requirements must be matched with the requirements of the encoded replicase, to ensure that the IVT-transcribed RNA can function efficiently as a substrate for its self-encoded replicase.

A self-replicating RNA can include (in addition to any 5′ cap structure) one or more nucleotides having a modified nucleobase. An RNA used with the invention ideally includes only phosphodiester linkages between nucleosides, but in some embodiments, it can contain phosphoramidate, and/or methylphosphonate linkages.

The self-replicating RNA molecule may encode a single heterologous polypeptide antigen (i.e. the antigen) or, optionally, two or more heterologous polypeptide antigens linked together in a way that each of the sequences retains its identity (e.g., linked in series) when expressed as an amino acid sequence. The heterologous polypeptides generated from the self-replicating RNA may then be produced as a fusion polypeptide or engineered in such a manner to result in separate polypeptide or peptide sequences.

The self-replicating RNA molecules described herein may be engineered to express multiple nucleotide sequences, from two or more open reading frames, thereby allowing co-expression of proteins, such as one, two or more antigens (e.g. one, two or more stem proteins) together with cytokines or other immunomodulators, which can enhance the generation of an immune response. Such a self-replicating RNA molecule might be particularly useful, for example, in the production of various gene products (e.g., proteins) at the same time, for example, as a bivalent or multivalent vaccine.

If desired, the self-replicating RNA molecules can be screened or analyzed to confirm their therapeutic and prophylactic properties using various in vitro or in vivo testing methods that are known to those of skill in the art. For example, vaccines comprising self-replicating RNA molecule can be tested for their effect on induction of proliferation or effector function of the particular lymphocyte type of interest, e.g., B cells, T cells, T cell lines, and T cell clones. For example, spleen cells from immunized mice can be isolated and the capacity of cytotoxic T lymphocytes to lyse autologous target cells that contain a self-replicating RNA molecule that encodes an antigen. In addition, T helper cell differentiation can be analyzed by measuring proliferation or production of TH1 (IL-2 and IFN-γ) and/or TH2 (IL-4 and IL-5) cytokines by ELISA or directly in CD4+ T cells by cytoplasmic cytokine staining and flow cytometry.

Self-replicating RNA molecules that encode an antigen can also be tested for ability to induce humoral immune responses, as evidenced, for example, by induction of B cell production of antibodies specific for the antigen of interest. These assays can be conducted using, for example, peripheral B lymphocytes from immunized individuals. Such assay methods are known to those of skill in the art. Other assays that can be used to characterize the self-replicating RNA molecules can involve detecting expression of the encoded antigen by the target cells. For example, FACS can be used to detect antigen expression on the cell surface or intracellularly. Another advantage of FACS selection is that one can sort for different levels of expression; sometimes-lower expression may be desired. Other suitable method for identifying cells which express a particular antigen involve panning using monoclonal antibodies on a plate or capture using magnetic beads coated with monoclonal antibodies.

In one embodiment the mRNA has the configuration 5′cap-5′UTR-non-structural proteins (NSP) 1-4-signal peptide-influenza HA stem polypeptide-linker-protein nanoparticle-3′UTR-polyA.

In some embodiments, the mRNA comprises or consists of a nucleic acid sequence having at least 90%, 95%, 98%, 99% or 100% identity to the nucleic sequence set forth in any one of SEQ ID NO: 20-22. In some embodiments, the mRNA comprises or consists of a nucleic acid sequence having at least 90%, 95%, 98%, 99% or 100% identity to the nucleic sequence set forth in any one of SEQ ID NO: 20. In some embodiments, the mRNA comprises or consists of a nucleic acid sequence having at least 90%, 95%, 98%, 99% or 100% identity to the nucleic sequence set forth in any one of SEQ ID NO: 22.

In some embodiments, the mRNA comprises or consists of a nucleic acid sequence having at least 90%, 95%, 98%, 99% or 100% identity to the nucleic sequence set forth in any one of SEQ ID NO: 23 or 24. In some embodiments, the mRNA comprises or consists of a nucleic acid sequence having at least 90%, 95%, 98%, 99% or 100% identity to the nucleic sequence set forth in any one of SEQ ID NO: 23.

A non-replicating mRNA will typically contain 10000 bases or fewer, especially 8000 bases or fewer, in particular 5000 base or fewer, especially 2500 bases or fewer. A replicating mRNA will typically contain 25000 bases or fewer, especially 20000 bases or fewer, in particular 15000 bases or fewer.

A single dose of mRNA may be 0.001 to 1000 ug, 0.01 to 1000 ug, especially 1 to 500 ug, in particular 10 to 250 ug of total mRNA. A single dose of mRNA may be 0.01 to 1 ug, especially 0.05 to 0.5 ug, in particular about 0.1 ug. A single dose of mRNA may be 0.1 to 10 ug, especially 0.5 to 5 ug, in particular about 1 ug. A single dose of mRNA may be 1 to 20 ug, especially 5 to 15 ug, in particular about 10 ug.

In one embodiment the mRNA is non-replicating mRNA. In a second embodiment the mRNA is replicating mRNA.

### Carriers

A range of carrier systems have been described which encapsulate or complex mRNA in order to facilitate mRNA delivery and consequent expression of encoded antigens as compared to mRNA which is not encapsulated or complexed. The present invention may utilise any suitable carrier system. Particular carrier systems of note are further described below.

### LNP

Lipid nanoparticles (LNPs) are non-virion liposome particles in which mRNA can be encapsulated. LNP delivery systems and methods for their preparation are known in the art. The particles can include some external mRNA (e.g. on the surface of the particles), but desirably at least half of the RNA (and suitably at least 85%, especially at least 95%, such as all of it) is encapsulated.

In some embodiments, the carrier is a lipid nanoparticle (LNP). In some embodiments, at least half of the mRNA is encapsulated in the LNP, suitably at least 85%, especially at least 95%, such as all of it.

LNP can, for example, be formed of a mixture of (i) a PEG-modified lipid (ii) a non-cationic lipid (iii) a sterol (iv) an ionisable cationic lipid. Alternatively, LNP can for example be formed of a mixture of (i) a PEG-modified lipid (ii) a non-cationic lipid (iii) a sterol (iv) a non-ionisable cationic lipid.

In some embodiments, the LNP comprises a PEG-modified lipid and a non-ionisable cationic lipid. In some embodiments, the LNP comprises a PEG-modified lipid and an ionisable cationic lipid.

The cationic lipid of an LNP may be cationisable, i.e. it becomes protonated as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

Such lipids include, but are not limited to, DSDMA, N,N-dioleyl-N,N-dimethylammonium chloride (DODAC), N,N-distearyl-N,N-dimethylammonium bromide (DDAB), 1,2-dioleoyltrimethyl ammonium propane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), ckk-E12, ckk, 1,2-DiLinoleyloxy-N, N-dimethylaminopropane (DLinDMA), 1,2-Dilinolenyloxy-N,N-dimethylaminopropane (DLenDMA), 1,2-di-y-linolenyloxy-N,N-dimethylaminopropane (y-DLenDMA), 98N12-5, 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA·Cl), ICE (Imidazol-based), HGT5000, HGT5001, DM DMA, CLinDMA, CpLinDMA, DMOBA, DOcarbDAP, DLincarbDAP, DLinCDAP, KLin-K-DMA, DLin-K-XTC2-DMA, XTC (2,2-Dilinoleyl-4-dimethylaminoethyl[1,3]-dioxolane) HGT4003, 1,2-Dilinoleoyl-3-trimethylaminopropane chloride salt (DLin-TAP·Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl-4-(dimethylamino)butanoate (MC3), ALNY-100 ((3aR,5s,6aS)-N,N-dimethyl-2,2-di((9Z,12Z)-octadeca-9,12-dienyl)tetrahydro-3aH-cyclopenta[d] [1,3]dioxol-5-amine)), 1,1′-(2-(4-(24(2-(bis(2-hydroxydodecyl)amino)ethyl)(2-hydroxydodecyl)amino)ethyl)piperazin-1-AethylazanediAdidodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl[1,3]-dioxolane (DLin-K-DMA), NC98-5 (4,7, 13-tris(3-oxo-3-(undecylamino)propyl)-N1,N 16-diundecyl-4,7, 10,13-tetraazahexadecane-1,16-diamide), (6Z,9Z,28Z,31Z)-heptatriaconta-6,9,28,31-tetraen-19-yl 4-(dimethylamino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31Z)-heptatriaconta-6,9,28,31-tetraen-19-yloxy)-N,N-dimethylbutan-1-amine (MC4 Ether), LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.) or any combination of any of the foregoing. Further suitable cationic lipids for use in the compositions and methods of the invention include those described in international patent publications WO2010/053572 (and particularly, Cl 2-200 described at paragraph [00225]) and WO2012/170930, both of which are incorporated herein by reference, HGT4003, HGT5000, HGTS001, HGT5001, HGT5002 (see US2015/0140070A1).

In embodiments, the cationic lipid may be an amino lipid.

Representative amino lipids include, but are not limited to, 1,2-dilinoleyoxy-3-(dimethylamino)acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA·Cl), 1,2-dilinoleoyl-3-trimethylaminopropane chloride salt (DLin-TAP·Cl), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA), 2,2-dilinoleyl-4-(2-dimethylaminoethyl)[1,3]-dioxolane (DLin-KC2-DMA); dilinoleyl-methyl-4-dimethylaminobutyrate (DLin-MC3-DMA); MC3 (US20100324120).

In embodiments, the cationic lipid may an aminoalcohol lipidoid.

Aminoalcohol lipidoids which may be used in the present invention may be prepared by the methods described in U.S. Pat. No. 8,450,298, herein incorporated by reference in its entirety. Suitable (ionizable) lipids can also be the compounds as disclosed in Tables 1, 2 and 3 and as defined in claims 1-24 of WO2017/075531A1, hereby incorporated by reference.

In another embodiment, suitable lipids can also be the compounds as disclosed in WO2015/074085A1 (i.e. ATX-001 to ATX-032 or the compounds as specified in claims 1-26), U.S. Appl. Nos. 61/905,724 and 15/614,499 or U.S. Pat. Nos. 9,593,077 and 9,567,296 hereby incorporated by reference in their entirety.

In other embodiments, suitable cationic lipids can also be the compounds as disclosed in WO2017/117530A1 (i.e. lipids 13, 14, 15, 16, 17, 18, 19, 20, or the compounds as specified in the claims), hereby incorporated by reference in its entirety.

In some embodiments, ionizable or cationic lipids may also be selected from the lipids disclosed in WO2018/078053A1 (i.e. lipids derived from formula I, II, and III of WO2018/078053A1, or lipids as specified in claims 1 to 12 of WO2018/078053A1), the disclosure of WO2018/078053A1 hereby incorporated by reference in its entirety. In that context, lipids disclosed in Table 7 of WO2018/078053A1 (e.g. lipids derived from formula I-1 to I-41) and lipids disclosed in Table 8 of WO2018/078053A1 (e.g. lipids derived from formula II-1 to II-36) may be suitably used herein. Accordingly, formula I-1 to formula I-41 and formula II-1 to formula II-36 of WO2018/078053A1, and the specific disclosure relating thereto, are herewith incorporated by reference.

In some embodiments, cationic lipids may be derived from formula III of published PCT patent application WO2018/078053A1. Accordingly, formula III of WO2018/078053A1, and the specific disclosure relating thereto, are herewith incorporated by reference.

In some embodiments, the at least one mRNA of the composition is complexed with one or more lipids thereby forming LNPs, wherein the cationic lipid of the LNP is selected from structures III-1 to III-36 of Table 9 of published PCT patent application WO2018/078053A1. Accordingly, formula III-1 to III-36 of WO2018/078053A1, and the specific disclosure relating thereto, are herewith incorporated by reference

In some embodiments, the LNP comprises a cationic lipid having the following structure:

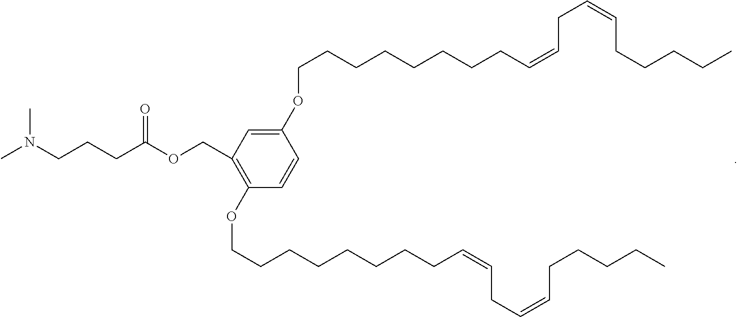 [SMILES: CCCCC/C=C\C/C=C\CCCCCCCCOc1ccc(OCCCCCCCC/C=C\C/C=C\CCCCC)c(COC(=O)CCCN(C)C)c1]

In some embodiments, the LNP comprises a cationic lipid according to formula III-3:

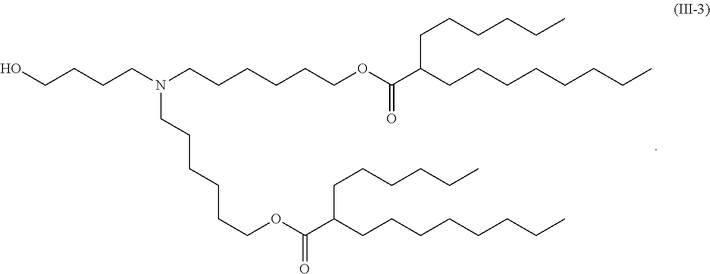 [SMILES: CCCCCCCCC(CCCCCC)C(=O)OCCCCCCN(CCCCO)CCCCCCOC(=O)C(CCCCCC)CCCCCCCC]

The lipid of formula III-3 as used herein in some embodiments has the chemical term ((4-hydroxybutypazanediyl)bis(hexane-6,1-diyl)bis(2-hexyldecanoate), also referred to as ALC-0315.

In certain embodiments, the cationic lipid as defined herein, such as the cationic lipid compound III-3, is present in the LNP in an amount from about 30 to about 95 mole percent, relative to the total lipid content of the LNP. If more than one cationic lipid is incorporated within the LNP, such percentages apply to the combined cationic lipids.

In embodiments, the cationic lipid is present in the LNP in an amount from about 30 to about 70 mole percent. In one embodiment, the cationic lipid is present in the LNP in an amount from about 40 to about 60 mole percent, such as about 40, 41, 42, 43, 44, 45, 46, 47, 48, 49, 50, 51, 52, 53, 54, 55, 56, 57, 58, 59 or 60 mole percent, respectively. In embodiments, the cationic lipid is present in the LNP in an amount from about 47 to about 48 mole percent, such as about 47.0, 47.1, 47.2, 47.3, 47.4, 47.5, 47.6, 47.7, 47.8, 47.9, 50.0 mole percent, respectively, wherein 47.7 mole percent are particularly suitable.

In some embodiments, the cationic lipid is present in a ratio of from about 20 mol % to about 70 or 75 mol % or from about 45 to about 65 mol % or about 20, 25, 30, 35, 40, 45, 50, 55, 60, 65, or about 70 mol % of the total lipid present in the LNP. In further embodiments, the LNPs comprise from about 25% to about 75% on a molar basis of cationic lipid, e.g., from about 20 to about 70%, from about 35 to about 65%, from about 45 to about 65%, about 60%, about 57.5%, about 57.1%, about 50% or about 40% on a molar basis (based upon 100% total moles of lipid in the lipid nanoparticle). In some embodiments, the ratio of cationic lipid to nucleic acid (e.g. coding RNA or DNA) is from about 3 to about 15, such as from about 5 to about 13 or from about 7 to about 11.

The PEG-modified lipid may comprise a PEG molecule with a molecular weight of 10000 Da or less, especially 5000 Da or less, in particular 3000 Da, such 2000 Da or less. Examples of PEG-modified lipids include PEG-distearoyl glycerol, PEG-dipalmitoyl glycerol and PEG-dimyristoyl glycerol. The PEG-modified lipid is typically present at around 0.5 to 15 molar %. In some embodiments, the LNPs comprise a polymer conjugated lipid. The term “polymer conjugated lipid” refers to a molecule comprising both a lipid portion and a polymer portion. An example of a polymer conjugated lipid is a PEGylated lipid. The term “PEGylated lipid” refers to a molecule comprising both a lipid portion and a polyethylene glycol portion. PEGylated lipids are known in the art and include 1-(monomethoxy-polyethyleneglycol)-2,3-dimyristoylglycerol (PEG-s-DMG) and the like.

In certain embodiments, the LNP comprises a stabilizing-lipid which is a polyethylene glycol-lipid (PEGylated lipid). Suitable polyethylene glycol-lipids include PEG-modified phosphatidylethanolamine, PEG-modified phosphatidic acid, PEG-modified ceramides (e.g. PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol)2000)carbamyl]-1,2-dimyristyloxlpropyl-3-amine (PEG-c-DMA). In an embodiment, the polyethylene glycol-lipid is PEG-2000-DMG. In one embodiment, the polyethylene glycol-lipid is PEG-c-DOMG). In other embodiments, the LNPs comprise a PEGylated diacylglycerol (PEG-DAG) such as 1-(monomethoxy-polyethyleneglycol)-2,3-dimyristoylglycerol (PEG-DMG), a PEGylated phosphatidylethanoloamine (PEG-PE), a PEG succinate diacylglycerol (PEG-S-DAG) such as 4-O-(2′, 3′-di(tetradecanoyloxy)propyl-1-O-(ω-methoxy(polyethoxy)ethyl)butanedioate (PEG-S-DMG), a PEGylated ceramide (PEG-cer), or a PEG dialkoxypropylcarbamate such as w-methoxy(polyethoxy)ethyl-N-(2,3di(tetradecanoxy)propyl)carbamate or 2,3-di(tetradecanoxy)propyl-N-(w-methoxy(polyethoxy)ethyl)carbamate.

In some embodiments, the LNP comprises a PEGylated lipid selected from PEG-PE and PEG-DMG. In some embodiments, the PEGylated lipid is PEG-DMG.

In some embodiments, the LNP comprises a PEG lipid of formula (IVa):

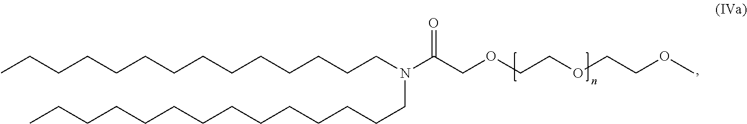

wherein n is an integer selected such that the average molecular weight of the PEG lipid is about 2500 g/mol.

In some embodiments, the LNP comprises a PEG lipid of formula (IVa):

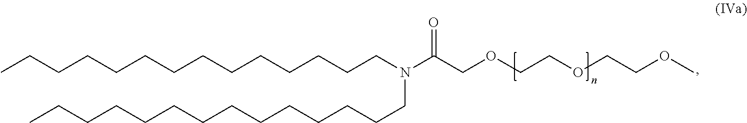

wherein n has a mean value ranging from 30 to 60, such as wherein n has a mean value of about 45, 46, 47, 48, 49, 50, 51, 52, 53, 54, such as wherein n has a mean value of 49 or 45.

The lipid of formula IVa as used herein in some embodiments has the chemical term 2[(polyethylene glycol)-2000]-N,N-ditetradecylacetamide, also referred to as ALC-0159.

In some embodiments, LNPs include less than about 3, 2, or 1 mole percent of PEG or PEG-modified lipid, based on the total moles of lipid in the LNP. In further embodiments, LNPs comprise from about 0.1% to about 20% of the PEG-modified lipid on a molar basis, e.g., about 0.5 to about 10%, about 0.5 to about 5%, about 10%, about 5%, about 3.5%, about 3%, about 2.5%, about 2%, about 1.5%, about 1%, about 0.5%, or about 0.3% on a molar basis (based on 100% total moles of lipids in the LNP). In some embodiments, LNPs comprise from about 1.0% to about 2.0% of the PEG-modified lipid on a molar basis, e.g., about 1.2 to about 1.9%, about 1.2 to about 1.8%, about 1.3 to about 1.8%, about 1.4 to about 1.8%, about 1.5 to about 1.8%, about 1.6 to about 1.8%, in particular about 1.4%, about 1.5%, about 1.6%, about 1.7%, about 1.8%, about 1.9%, most suitably 1.7% (based on 100% total moles of lipids in the LNP).

In some embodiments, the LNP comprises one or more additional lipids, (e.g. a non-cationic lipid and/or one or more steroid or steroid analogue, such as a sterol).

In some embodiments, the LNP comprises one or more non-cationic lipid, such as neutral lipids and/or one or more steroid or steroid analogues, such as sterol.

In some embodiments, the LNP comprises a PEG-modified lipid, optionally a non-cationic lipid, optionally a sterol and a non-ionisable cationic lipid. In some embodiments, the LNP comprises a PEG-modified lipid, optionally a non-cationic lipid, optionally a sterol and an ionisable cationic lipid.

The non-cationic lipid may be a neutral lipid, such as 1,2-distearoyl-sn-glycero-3-phosphocholine (DSPC), 1,2-dipalmitoyl-sn-glycero-3-phosphocholine (DPPC), 1-palmitoyl-2-oleoyl-sn-glycero-3-phosphocholine (POPC), 1,2-dioleoyl-sn-glycero-3-phosphoethanolamine (DOPE) and sphingomyelin (SM). The non-cationic lipid is typically present at around 5 to 25 molar %.

The sterol may be cholesterol. The sterol is typically present at around 25 to 55 molar %.

A range of suitable ionizable cationic lipids are known in the art, which are typically present at around 20 to 60 molar %.

In some embodiments, the LNP comprise a PEG-modified lipid at around 0.5 to 15 molar %, a non-cationic lipid at around 5 to 25 molar %, a sterol at around 25 to 55 molar % and an ionisable cationic lipid at around 20 to 60 molar %.

In some embodiments, the molar ratio of the cationic lipid to cholesterol is in the range from about 2:1 to about 1:1.

In some embodiments, the neutral lipid is 1,2-distearoyl-sn-glycero-3-phosphocholine (DSPC). In some embodiments, the neutral lipid is 1,2-distearoyl-sn-glycero-3-phosphocholine (DSPC) wherein the molar ratio of the cationic lipid to DSPC is in the range from about 2:1 to about 8:1.

In one embodiment the lipid nanoparticles comprise about 40% cationic lipid LKY750, about 10% zwitterionic lipid DSPC, about 48% cholesterol, and about 2% PEGylated lipid DMG (w/w).

The ratio of RNA to lipid can be varied (see for example WO2013/006825). “N:P ratio” refers to the molar ratio of protonatable nitrogen atoms in the cationic lipids (typically solely in the lipid's headgroup) to phosphates in the RNA. The ratio of nucleotide (N) to phospholipid (P) can be in the range of, e.g., 1N:1P to 20N:1P, 1N:1P to 10N:1P, 2N:1P to 8N:1P, 2N:1P to 6N:1P or 3N:1P to 5N:1P. The ratio of nucleotide (N) to phospholipid (P) can be in the range of, e.g., 1N:1P, 2N:1P, 3N:1P, 4N:1P, 5N:1P, 6N:1P, 7N:1P, 8N:1P, 9N:1P, or 10N:1P. Alternatively, or additionally, the ratio of nucleotide (N) to phospholipid (P) is 4N:1P. In some embodiments, the ratio of nucleotide (N) to phospholipid (P) is in the range of 1N:1P to 20N:1P, 1N:1P to 10N:1P, 2N:1P to 8N:1P, 2N:1P to 6N:1P or 3N:1P to 5N:1P.

WO2017/070620 provides general information on LNP compositions and is incorporated herein by reference. Other useful LNPs are described in the following references: WO2012/006376; WO2012/030901; WO2012/031046; WO2012/031043; WO2012/006378; WO2011/076807; WO2013/033563; WO2013/006825; WO2014/136086; WO2015/095340; WO2015/095346; WO2016/037053, which are also incorporated herein by reference.

LNPs are typically 50 to 200 nm in diameter (Z-average). Suitably the LNPs have a polydispersity of 0.4 or less, such as 0.3 or less.

### CNE

The carrier may be a cationic nanoemulsion (CNE) delivery system. Such cationic oil-in-water emulsions can be used to deliver the mRNA to the interior of a cell. The emulsion particles comprise a hydrophobic oil core and a cationic lipid, the latter of which can interact with the mRNA, thereby anchoring it to the emulsion particle. In a CNE delivery system, the mRNA which encodes the antigen is complexed with a particle of a cationic oil-in-water emulsion. CNE carriers and methods for their preparation are described in WO2012/006380, WO2013/006837 and WO2013/006834 which are incorporated herein by reference.

Thus, the mRNA may be complexed with a particle of a cationic oil-in-water emulsion. The particles typically comprise an oil core (e.g. a plant oil or squalene) that is in liquid phase at 25° C., a cationic lipid (e.g. phospholipid) and, optionally, a surfactant (e.g. sorbitan trioleate, polysorbate 80); polyethylene glycol can also be included. Alternatively or additionally, the CNE comprises squalene and a cationic lipid, such as 1,2-dioleoyloxy-3-(trimethylammonio)propane (DOTAP) (see e.g. Brito, 2014). In an embodiment, the CNE is an oil-in-water emulsion of DOTAP and squalene stabilised with polysorbate 80 and/or sorbitan trioleate.

Desirably at least half of the RNA (and suitably at least 85%, such as all of it) is complexed with the cationic oil-in-water emulsion carrier.

CNE are typically 50 to 200 nm in diameter (Z-average). Suitably the CNE have a polydispersity of 0.4 or less, such as 0.3 or less.

In one embodiment the carrier is a cationic nanoemulsion (CNE). In some embodiments, the CNE is an oil-in-water emulsion of DOTAP and squalene stabilised with polysorbate 80 and/or sorbitan trioleate.

### LION

A lipidoid-coated iron oxide nanoparticle (LION) is capable of delivering mRNA into cells and may be aided after administration to a subject by application of an external magnetic field. A LION is an iron oxide a particle with one or more coatings comprising lipids and/or lipidoids wherein mRNA encoding the antigen is incorporated into or associated with the lipid and/or lipidoid coating(s) through electrostatic interactions. The mRNA being embedded within the coating(s) may offer protection from enzymatic degradation. The lipids and/or lipidoids comprised within a LION may for example include those included in Figure S1 of Jiang, 2013, especially lipidoids comprising alkyl tails of 12 to 14 carbons in length and in particular lipidoid C14-200 as disclosed in Jiang, 2013. A LION may typically comprise 200 to 5000, such as 500 to 2000, in particular about 1000 about 1000 lipid and/or lipidoid molecules. Typically the LIONs are 20 to 200 nm in diameter, especially 50 to 100 nm in diameter. The lipid/lipidoid to mRNA weight ratio may be about 1:1 to 10:1, especially about 5:1. Particularly suitable LIONs, and methods for preparation of LIONs are disclosed in Jiang, 2013.

In one embodiment the carrier is a lipidoid-coated iron oxide nanoparticle (LION).

### Assays

The in vitro efficacy of vaccines which target the head region may be established by assays which investigate whether or not the vaccine prevents influenza virus from binding to target cells. An example of such an assay is the hemagglutination inhibition (HAI) assay, which is considered to be the gold standard in the field, and which provides a correlate of protection in vivo. However, vaccines which target the stem region, while being potentially protective, may not prevent influenza virus from binding to target cells. The above assays are therefore inappropriate for investigating the efficacy of a vaccine targeting the stem region.

Suitable assays for investigating the efficacy of a vaccine targeting the stem region which has been administered to mice are as follows. Implementations of these assays are used in the examples provided herein.

### Anti-HA IgG Antibodies by ELISA

Quantification of mouse anti-HA IgG antibodies are performed by ELISA using HA antigen (full length or stem only) as coating. The plates are then incubated. Diluted sera are added to the coated plates and incubated. The plates are washed prior to the adding of diluted peroxidase conjugated goat anti-mouse IgG. The reaction is stopped with H₂SO₄ and optical densities are read. The titers are expressed as ELISA Units Titers.

### Stem Specific T Cell Frequencies

Spleens are collected and cell suspensions are prepared. The splenic cell suspensions are filtered, harvested and centrifuged. Fresh splenocytes are then plated in the presence of an overlapping peptide pool covering the sequence of stem protein. Following stimulation, cells are washed and stained with anti-CD16/32, anti-CD4-V450 and anti-CD8-PerCp-Cy5.5 antibodies. Living/dead cell stain is added. Cells are permeabilized and stained with anti-IL2-FITC, anti-IFNγ-APC and anti-TNFα-PE antibodies. Stained cells are analyzed by flow cytometry.

### Neutralization Antibody Titers

Mouse sera are diluted and incubated in the presence of reporter influenza virus. After incubation, the serum-virus mix is added to cell culture. Influenza-positive cells are analysed and quantified by flow cytometry. Titers are expressed as 50% neutralization titers (IC50), corresponding to reduction titers calculated by regression analysis of the inverse dilution of serum that provides 50% cell infected reduction compared to control wells (virus only, no serum).

More specific implementations of the above assays are detailed in the examples. These more specific assays may also be used for investigating the efficacy of a vaccine targeting the stem region.

### Subjects

The present invention is generally intended for mammalian subjects, in particular human subjects. The subject may be a wild or domesticated animal. Mammalian subjects include for example cats, dogs, pigs, sheep, horses or cattle. In one embodiment, the subject is human.

The subject to be treated using the method of the invention may be of any age.

In one embodiment the subject is a human infant (up to 12 months of age). In one embodiment the subject is a human child (less than 18 years of age). In one embodiment the subject is an adult human (aged 18-59). In one embodiment the subject is an older human (aged 60 or greater).

Doses administered to younger children, such as less than 12 years of age, may be reduced relative to an equivalent adult dose, such as by 50%.

The methods of the invention are suitably intended for prophylaxis, i.e. for administration to a subject which is not infected with influenza virus.

### Formulation and Administration

The carrier-formulated mRNA may be administered via various suitable routes, including parenteral, such as intramuscular or subcutaneous administration. Suitably the carrier-formulated mRNA is administered intramuscularly.

The carrier-formulated mRNA may be provided in liquid or dry (e.g. lyophilised) form. The preferred form will depend on factors such as the precise nature of the carrier-formulated mRNA, e.g. if the carrier-formulated mRNA is amenable to drying, or other components which may be present.

The carrier-formulated mRNA is typically provided in liquid form.

In embodiments, the carrier-formulated mRNA may be lyophilized or spray-dried or spray-freeze dried.

A composition comprising carrier-formulated mRNA intended for combination with other compositions prior to administration need not itself have a physiologically acceptable pH or a physiologically acceptable tonicity; a formulation intended for administration should have a physiologically acceptable pH and should have a physiologically acceptable osmolality.

The pH of a liquid preparation is adjusted in view of the components of the composition and necessary suitability for administration to the human subject. The pH of a formulation is generally at least 4, especially at least 5, in particular at least 5.5 such as at least 6. The pH of a formulation is generally 9 or less, especially 8.5 or less, in particular 8 or less, such as 7.5 or less. The pH of a formulation may be 4 to 9, especially 5 to 8.5, in particular 5.5 to 8, such as 6.5 to 7.4 (e.g. 6.5 to 7.1).

For parenteral administration, solutions should have a physiologically acceptable osmolality to avoid excessive cell distortion or lysis. A physiologically acceptable osmolality will generally mean that solutions will have an osmolality which is approximately isotonic or mildly hypertonic. Suitably the formulations for administration will have an osmolality of 250 to 750 mOsm/kg, especially 250 to 550 mOsm/kg, in particular 270 to 500 mOsm/kg, such as 270 to 400 mOsm/kg. Osmolality may be measured according to techniques known in the art, such as by the use of a commercially available osmometer, for example the Advanced® Model 2020 available from Advanced Instruments Inc. (USA).

Liquids used for reconstitution will be substantially aqueous, such as water for injection, phosphate buffered saline and the like. As mentioned above, the requirement for buffer and/or tonicity modifying agents will depend on the on both the contents of the container being reconstituted and the subsequent use of the reconstituted contents. Buffers may be selected from acetate, citrate, histidine, maleate, phosphate, succinate, tartrate and TRIS. The buffer may be a phosphate buffer such as Na/Na₂PO₄, Na/K₂PO₄ or K/K₂PO₄.

Suitably, the formulations used in the present invention have a dose volume of between 0.05 ml and 1 ml, such as between 0.1 and 0.6 ml, in particular a dose volume of 0.45 to 0.55 ml, such as 0.5 ml. The volumes of the compositions used may depend on the subject, delivery route and location, with smaller doses being given by the intradermal route. A typical human dose for administration through routes such as intramuscular, is in the region of 200 ul to 750 ml, such as 400 to 600 ul, in particular about 500 ul, such as 500 ul.

The carrier-formulated mRNA may be provided in various physical containers such as vials or pre-filled syringes.

In some embodiments the carrier-formulated mRNA is provided in the form of a single dose. In other embodiments the carrier-formulated mRNA is provided in multidose form such containing 2, 5 or 10 doses.

It is common where liquids are to be transferred between containers, such as from a vial to a syringe, to provide ‘an overage’ which ensures that the full volume required can be conveniently transferred. The level of overage required will depend on the circumstances but excessive overage should be avoided to reduce wastage and insufficient overage may cause practical difficulties. Overages may be of the order of 20 to 100 ul per dose, such as 30 ul or 50 ul.

Stabilisers may be present. Stabilisers may be of particular relevance where multidose containers are provided as doses of the final formulation(s) may be administered to subjects over a period of time.

Formulations are preferably sterile.

Approaches for establishing strong and lasting immunity often include repeated immunisation, i.e. boosting an immune response by administration of one or more further doses. Such further administrations may be performed with the same immunogenic compositions (homologous boosting) or with different immunogenic compositions (heterologous boosting). The present invention may be applied as part of a homologous or heterologous prime/boost regimen, as either the priming or a/the boosting immunisation.

Administration of the carrier-formulated mRNA may therefore be part of a multi-dose administration regime. For example, the carrier-formulated mRNA may be provided as a priming dose in a multidose regime, especially a two- or three-dose regime, in particular a two-dose regime. The carrier-formulated mRNA may be provided as a boosting dose in a multidose regime, especially a two- or three-dose regime, such as a two-dose regime.

Priming and boosting doses may be homologous or heterologous. Consequently, the carrier-formulated mRNA may be provided as a priming dose and boosting dose(s) in a homologous multidose regime, especially a two- or three-dose regime, in particular a two-dose regime. Alternatively, the carrier-formulated mRNA may be provided as a priming dose or boosting dose in a heterologous multidose regime, especially a two- or three-dose regime, in particular a two-dose regime, and the boosting dose(s) may be different (e.g. carrier-formulated mRNA; or an alternative antigen presentation such as protein or virally vectored antigen—with or without adjuvant, such as squalene emulsion adjuvant).

The time between doses may be two weeks to six months, such as three weeks to three months. Periodic longer-term booster doses may be also be provided, such as every 2 to 10 years.

Also provided is an immunogenic composition comprising the carrier-formulated mRNA as described herein, wherein the composition optionally comprises at least one pharmaceutically acceptable carrier.

The at least one pharmaceutically acceptable carrier or excipient of the immunogenic composition may be selected to be suitable for intramuscular or intradermal delivery/administration of said immunogenic composition. The immunogenic composition is in some embodiments a composition suitable for intramuscular administration to a subject.

Subjects to which administration of the immunogenic compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

In some embodiments, the composition is a multivalent composition comprising a plurality or at least one further mRNA in addition to the mRNA as described herein.

In some embodiments, the multivalent composition comprises two or more mRNA as defined herein. In some embodiments, the multivalent composition comprises two mRNA as defined herein. In some embodiments, the multivalent composition comprises two or more mRNA as defined herein, such as two mRNA as defined herein, each encoding a different influenza HA stem polypeptide.

In some embodiments, the two or more mRNA encode influenza HA stem polypeptides derived from influenza A, such as influenza A Group 1 and/or influenza A Group 2.

In some embodiments, at least one of the two or more mRNA encodes an influenza HA stem polypeptide derived from influenza A Group 1, such as influenza A subtype H1, H2, H5, H6, H8, H9, H11, H12, H13, H16, H17 and/or H18; and at least one of the two or more mRNA encodes an influenza HA stem polypeptide derived from influenza A Group 2, such as influenza A subtype H3, H4, H7, H10, H14 and/or H15.

In some embodiments, at least one of the two or more mRNA encodes an influenza HA stem polypeptide derived from influenza A H1; and at least one of the two or more mRNA encodes an influenza HA stem polypeptide derived from influenza A H3 or H10.

In some embodiments, at least one of the two or more mRNA encodes an influenza HA stem polypeptide derived from influenza A H1; and at least one of the two or more mRNA encodes an influenza HA stem polypeptide derived from influenza A H10.

In some embodiments, the influenza HA stem polypeptide derived from influenza A Group 1 comprises or consists of an amino acid sequence having at least 90%, 95%, 98% or 99% identity to the amino acid sequence set forth in any one of SEQ ID NO:1 or SEQ ID NO: 2. In some embodiments, the influenza HA stem polypeptide derived from influenza A Group 1 comprises or consists of an amino acid sequence having at least 90%, 95%, 98% or 99% identity to the amino acid sequence set forth in SEQ ID NO: 2.

In some embodiments, the influenza HA stem polypeptide derived from influenza A Group 1 comprises or consists of the amino acid sequence set forth in any one of SEQ ID NO:1 or SEQ ID NO: 2. In some embodiments, the influenza HA stem polypeptide derived from influenza A Group 1 comprises or consists of the amino acid sequence set forth SEQ ID NO: 2.

In some embodiments, the influenza HA stem polypeptide derived from influenza A Group 2 comprises or consists of an amino acid sequence having at least 90%, 95%, 98% or 99% identity to the amino acid sequence set forth in any one of SEQ ID NO: 3 or SEQ ID NO: 4. In some embodiments, the influenza HA stem polypeptide derived from influenza A Group 2 comprises or consists of an amino acid sequence having at least 90%, 95%, 98% or 99% identity to the amino acid sequence set forth in SEQ ID NO: 4.

In some embodiments, the influenza HA stem polypeptide derived from influenza A Group 2 comprises or consists of the amino acid sequence set forth in any one of SEQ ID NO: 3 or SEQ ID NO: 4. In some embodiments, the influenza HA stem polypeptide derived from influenza A Group 2 comprises or consists of the amino acid sequence set forth in SEQ ID NO: 4.

In some embodiments, at least one of the two or more mRNA are self-replicating. In some embodiments, each of the two or more mRNA are self-replicating.

Also provided is a vaccine comprising the mRNA and/or the immunogenic composition as defined herein.

In some embodiments, the vaccine is a multivalent vaccine comprising a plurality or at least more than one of the RNA as described herein, or a plurality or at least more than one of the composition as defined herein.

Also provided is a kit or kit of parts comprising the RNA, and/or the composition, and/or the vaccine as defined herein, optionally comprising a liquid vehicle for solubilising, and, optionally, technical instructions providing information on administration and dosage of the components.

The technical instructions of said kit may contain information about administration and dosage and patient groups. Such kits, suitably kits of parts, may be applied e.g. for any of the applications or uses mentioned herein, such as for the use of the immunogenic composition of the invention or the vaccine of the invention, for the treatment or prophylaxis of an infection or diseases caused by an Influenza virus, suitably Influenza A virus.

In some embodiments, the immunogenic composition or the vaccine is provided in a separate part of the kit, wherein the immunogenic composition or the vaccine is suitably lyophilised or spray-dried or spray-freeze dried.

The kit may further contain as a part a vehicle (e.g. buffer solution) for solubilising the dried or lyophilized nucleic composition or the vaccine.

Any of the above kits may be used in a treatment or prophylaxis as defined herein.

Also provided is the carrier-formulated mRNA, the immunogenic composition, the vaccine or the kit or kit of parts as defined herein for use as a medicament.

Also provided is the carrier-formulated mRNA, the immunogenic composition, the vaccine or the kit or kit of parts as described herein for use in the treatment or prophylaxis of an infection with an influenza virus.

Also provided is the carrier-formulated mRNA, the immunogenic composition, the vaccine or the kit or kit of parts as described herein for use in the treatment or prophylaxis of an infection with an influenza A virus.

In some embodiments, the amount of carrier-formulated mRNA for each carrier-formulated mRNA is essentially equal in mass. In other embodiments, the amount of nucleic acid for each nucleic acid species is selected to be equimolar.

In some embodiments, a single dose of the carrier-formulated mRNA is 0.001 to 1000 μg, 0.01 to 1000 μg, especially 1 to 500 μg, in particular 10 to 250 μg of total mRNA.

In some embodiments, the carrier-formulated mRNA, the immunogenic composition, the vaccine, the kit or kit of parts for use as defined herein is for intramuscular administration. In some embodiments, the carrier-formulated mRNA, the immunogenic composition, the vaccine, the kit or kit of parts for use as defined herein is for intradermal administration.

In some embodiments, an immune response is elicited. In some embodiments, an adaptive immune response is elicited. In some embodiments, a protective adaptive immune response against an influenza virus is elicited. In some embodiments, a protective adaptive immune response against an influenza A virus is elicited. In some embodiments, a protective adaptive immune response against one or more influenza A virus subtype from Group 1 and/or Group 2 is elicited.

In some embodiments, the elicited immune response comprises neutralizing antibody titers against an influenza virus. In some embodiments, the elicited immune response comprises neutralizing antibody titers against an influenza A virus. In some embodiments, the elicited immune response comprises neutralizing antibody titers against one or more influenza A virus subtype from Group 1 and/or Group 2.

In some embodiments, the elicited immune response comprises functional antibodies that can effectively neutralize the respective viruses.

In some embodiments, the elicited immune response comprises broad, functional cellular T-cell responses against the respective viruses. In particular, the elicited immune response comprises a CD4+ T cell immune response and/or a CD8+ T cell immune response.

In some embodiments, the elicited immune response comprises a well-balanced B cell and T cell response against the respective viruses.

In some embodiments, the elicited immune response comprises antigen-specific immune responses.

In some embodiments, the elicited immune response reduces partially or completely the severity of one or more symptoms and/or time over which one or more symptoms of influenza virus infection are experienced by the subject.

In some embodiments, the elicited immune response reduces the likelihood of developing an established influenza virus infection after challenge.

In some embodiments, the elicited immune response slows progression of influenza.

Also provided is a method of treating or preventing a disorder, wherein the method comprises applying or administering to a subject in need thereof the carrier-formulated mRNA, the composition, the vaccine or the kit or kit of parts as defined herein.

Preventing (Inhibiting) or treating a disease, in particular a virus infection relates to inhibiting the full development of a disease or condition, for example, in a subject who is at risk for a disease such as a virus infection. “Treatment” refers to a therapeutic intervention that ameliorates a sign or symptom of a disease or pathological condition after it has begun to develop. The term “ameliorating”, with reference to a disease or pathological condition, refers to any observable beneficial effect of the treatment. Inhibiting a disease can include preventing or reducing the risk of the disease, such as preventing or reducing the risk of viral infection. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology.

In some embodiments, the carrier-formulated mRNA, the composition, the vaccine or the kit or kit of parts of the invention is administered at a therapeutically effective amount.

In some embodiments, the disorder is an infection with an influenza virus. In some embodiments, the disorder is an infection with an influenza A virus.

In some embodiments, the subject in need is a mammalian subject, such as a human subject.

Also provided is a method of eliciting an immune response, wherein the method comprises applying or administering to a subject in need thereof the carrier-formulated mRNA, the composition, the vaccine or the kit or kit as defined herein.

In some embodiments, the immune response is an adaptive immune response. In some embodiments, the immune response is a protective adaptive immune response against an influenza virus. In some embodiments, the immune response is a protective adaptive immune response against an influenza A virus. In some embodiments, the immune response is a protective adaptive immune response against an influenza A virus one or more influenza A virus subtype from Group 1 and/or Group 2.

In embodiments, the elicited immune response comprises functional antibodies that can effectively neutralize the respective viruses.

In further embodiments, the elicited immune response comprises broad, functional cellular T-cell responses against the respective viruses.

In further embodiments, the elicited immune response comprises a well-balanced B cell and T cell response against the respective viruses.

In some embodiments, the immune response comprises a homologous, a heterologous and/or a heterosubtypic cross-reactive immunogenic responses against Influenza virus, such as against Influenza A virus, such as against Influenza A virus subtypes of Group 1 and/or Group 2.

In some embodiments, the subject in need is a mammalian subject, such as a human subject.

In embodiments, administration of the carrier-formulated mRNA, the composition, the vaccine or the kit or kit of the invention to a subject elicits neutralizing antibodies and does not elicit disease enhancing antibodies. In particular, administration of the carrier-formulated mRNA, the composition, the vaccine or the kit or kit of the invention to a subject does not elicit immunopathological effects, like e.g. enhanced disease and/or antibody dependent enhancement (ADE).

It has to be noted that specific features and embodiments that are described in the context of the carrier-formulated mRNA and/or the immunogenic composition are likewise applicable to the vaccine, the kit or kit of parts or further aspects including e.g. medical uses (first and second medical uses) and e.g. method of treatments.

### Definitions

Throughout the specification, including the claims, where the context permits, the term “comprising” and variants thereof such as “comprises” are to be interpreted as including the stated element (e.g., integer) or elements (e.g., integers) without necessarily excluding any other elements (e.g., integers). Thus, a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in or “approximately” in relation to a numerical value x is optional and means, for example, x±10% of the given figure, such as x±5% of the given figure.

As used herein, the singular forms “a,” “an” and “the” include plural references unless the content clearly dictates otherwise.

Unless specifically stated, a process comprising a step of mixing two or more components does not require any specific order of mixing. Thus components can be mixed in any order. Where there are three components then two components can be combined with each other, and then the combination may be combined with the third component, etc.

Percentages in the context of numbers should be understood as relative to the total number of the respective items. In other cases, and unless the context dictates otherwise, percentages should be understood as percentages by weight (wt.-%).

Adaptive immune response: The term “adaptive immune response” as used herein will be recognized and understood by the person of ordinary skill in the art, and is e.g. intended to refer to an antigen-specific response of the immune system (the adaptive immune system). Antigen specificity allows for the generation of responses that are tailored to specific pathogens or pathogen-infected cells. The ability to mount these tailored responses is usually maintained in the body by “memory cells” (B-cells).

Antigen: The term “antigen” as used herein will be recognized and understood by the person of ordinary skill in the art, and is e.g. intended to refer to a substance which may be recognized by the immune system, suitably by the adaptive immune system, and is capable of triggering an antigen-specific immune response, e.g. by formation of antibodies and/or antigen-specific T cells as part of an adaptive immune response. Typically, an antigen may be or may comprise a peptide or protein, which may be presented by the MHC to T-cells. Also fragments, variants and derivatives of peptides or proteins comprising at least one epitope are understood as antigens in the context of the invention. In the context of the present invention, an antigen may be the product of translation of a provided RNA as specified herein.

Antigenic peptide or protein: The term “antigenic peptide or protein” or “immunogenic peptide or protein” will be recognized and understood by the person of ordinary skill in the art, and is e.g. intended to refer to a peptide, protein derived from a (antigenic or immunogenic) protein which stimulates the body's adaptive immune system to provide an adaptive immune response. Therefore, an antigenic/immunogenic peptide or protein comprises at least one epitope or antigen of the protein it is derived from.

Cationic: Unless a different meaning is clear from the specific context, the term “cationic” means that the respective structure bears a positive charge, either permanently or not permanently, but in response to certain conditions such as pH. Thus, the term “cationic” covers both “permanently cationic” and “cationisable”.

Cationisable: The term “cationisable” as used herein means that a compound, or group or atom, is positively charged at a lower pH and uncharged at a higher pH of its environment. Also, in non-aqueous environments where no pH value can be determined, a cationisable compound, group or atom is positively charged at a high hydrogen ion concentration and uncharged at a low concentration or activity of hydrogen ions. It depends on the individual properties of the cationisable or polycationisable compound, in particular the pKa of the respective cationisable group or atom, at which pH or hydrogen ion concentration it is charged or uncharged. In diluted aqueous environments, the fraction of cationisable compounds, groups or atoms bearing a positive charge may be estimated using the so-called Henderson-Hasselbalch equation, which is well-known to a person skilled in the art. E.g., in some embodiments, if a compound or moiety is cationisable, it is suitable that it is positively charged at a pH value of about 1 to 9, such as 4 to 9, 5 to 8 or even 6 to 8, more suitably of a pH value of or below 9, of or below 8, of or below 7, most suitably at physiological pH values, e.g. about 7.3 to 7.4, i.e. under physiological conditions, particularly under physiological salt conditions of the cell in vivo. In other embodiments, it is suitable that the cationisable compound or moiety is predominantly neutral at physiological pH values, e.g. about 7.0-7.4, but becomes positively charged at lower pH values. In some embodiments, the suitable range of pKa for the cationisable compound or moiety is about 5 to about 7.

Coding sequence/coding region: The terms “coding sequence” or “coding region” and the corresponding abbreviation “cds” as used herein will be recognized and understood by the person of ordinary skill in the art, and are e.g. intended to refer to a sequence of several nucleotide triplets, which may be translated into a peptide or protein. A coding sequence in the context of the present invention may be an RNA sequence consisting of a number of nucleotides that may be divided by three, which starts with a start codon and which suitably terminates with a stop codon.

Epitope: The term “epitope” (also called “antigen determinant” in the art) as used herein will be recognized and understood by the person of ordinary skill in the art, and is e.g. intended to refer to T cell epitopes and B cell epitopes. T cell epitopes or parts of the antigenic peptides or proteins and may comprise fragments suitably having a length of about 6 to about 20 or even more amino acids, e.g. fragments as processed and presented by MHC class I molecules, suitably having a length of about 8 to about 10 amino acids, e.g. 8, 9, or 10, (or even 11, or 12 amino acids), or fragments as processed and presented by MHC class II molecules, suitably having a length of about 13 to about 20 or even more amino acids. These fragments are typically recognized by T cells in form of a complex consisting of the peptide fragment and an MHC molecule, i.e. the fragments are typically not recognized in their native form. B cell epitopes are typically fragments located on the outer surface of (native) protein or peptide antigens, suitably having 5 to 15 amino acids, more preferably having 5 to 12 amino acids, even more suitably having 6 to 9 amino acids, which may be recognized by antibodies, i.e. in their native form. Such epitopes of proteins or peptides may furthermore be selected from any of the herein mentioned variants of such proteins or peptides. In this context epitopes can be conformational or discontinuous epitopes which are composed of segments of the proteins or peptides as defined herein that are discontinuous in the amino acid sequence of the proteins or peptides as defined herein but are brought together in the three-dimensional structure or continuous or linear epitopes which are composed of a single polypeptide chain.

Fragment: The term “fragment” as used throughout the present specification in the context of a nucleic acid sequence (e.g. RNA or a DNA) or an amino acid sequence may typically be a shorter portion of a full-length sequence of e.g. a nucleic acid sequence or an amino acid sequence, while still retaining its intended function. Accordingly, a fragment, typically, consists of a sequence that is identical to the corresponding stretch within the full-length sequence. A suitable fragment of a sequence, consists of a continuous stretch of entities, such as nucleotides or amino acids corresponding to a continuous stretch of entities in the molecule the fragment is derived from, which represents at least 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, 99.5% of the total (i.e. full-length) molecule from which the fragment is derived. The term “fragment” as used throughout the present specification in the context of proteins or peptides may, typically, comprise a sequence of a protein or peptide as defined herein, which is, with regard to its amino acid sequence, N-terminally and/or C-terminally truncated compared to the amino acid sequence of the original protein. Such truncation may thus occur either on the amino acid level or correspondingly on the nucleic acid level. A sequence identity with respect to such a fragment as defined herein may therefore suitably refer to the entire protein or peptide as defined herein or to the entire (coding) nucleic acid molecule of such a protein or peptide. Fragments of proteins or peptides may comprise at least one epitope of those proteins or peptides.

Humoral immune response: The terms “humoral immunity” or “humoral immune response” will be recognized and understood by the person of ordinary skill in the art, and are e.g. intended to refer to B-cell mediated antibody production and optionally to accessory processes accompanying antibody production. A humoral immune response may be typically characterized, e.g. by Th2 activation and cytokine production, germinal center formation and isotype switching, affinity maturation and memory cell generation. Humoral immunity may also refer to the effector functions of antibodies, which include pathogen and toxin neutralization, classical complement activation, and opsonin promotion of phagocytosis and pathogen elimination.

Immunogen, immunogenic: The terms “immunogen” or “immunogenic” will be recognized and understood by the person of ordinary skill in the art, and are e.g. intended to refer to a compound that is able to stimulate/induce an immune response. An immunogen may be a peptide, polypeptide, or protein. An immunogen in the sense of the present invention is the product of translation of a provided RNA comprising at least one coding sequence encoding at least one antigenic peptide, protein, polypeptide. Typically, an immunogen elicits an adaptive immune response.

Immune response: The term “immune response” will be recognized and understood by the person of ordinary skill in the art, and is e.g. intended to refer to a specific reaction of the adaptive immune system to a particular antigen (so called specific or adaptive immune response) or an unspecific reaction of the innate immune system (so called unspecific or innate immune response), or a combination thereof. A suitable vaccine induces an efficient immune response in a normal healthy recipient to whom the vaccine is administered. With an efficient immune response one vaccination will result in virus-neutralizing antibody titers. In addition, or alternatively, an efficient immune response will elicit an adaptive immune response. In some embodiments the efficient immune response will reduce Influenza infection by at least 50% relative to a neutralizing antibody titer of an unvaccinated control subject. In some embodiments, an efficient immune response will be one where the neutralizing antibody titer and/or a T cell immune response is sufficient to reduce the rate of asymptomatic viral infection relative to the neutralizing antibody titer of unvaccinated control subjects. An efficient immune response may also be one where the neutralizing antibody titer and/or a T cell immune response is sufficient to prevent viral latency in the subject and/or the neutralizing antibody titer is sufficient to block fusion of virus with epithelial cells of the subject. In some embodiments an efficient immune response is one in which administration of a therapeutically effective amount of the nucleic acid, the composition, or the vaccine to a subject induces a T cell immune response against coronavirus in the subject. In some embodiments, the T cell immune response comprises a CD4+ T cell immune response and/or a CD8+ T cell immune response. In further aspects, an efficient immune response is one in which the immune response protects the subject from Influenza disease for at least about 6 months and/or reduce the incidence of hospitalization compared to an unvaccinated person. An efficient immune response may also reduce the transmission of virus due compared to transmission from an unvaccinated person infected with the virus. An efficient immune response may also be considered as one which provide some protection against variants due to heterologous immune responses.

T-cell responses: The terms “cellular immunity” or “cellular immune response” or “cellular T-cell responses” as used herein will be recognized and understood by the person of ordinary skill in the art, and are for example intended to refer to the activation of macrophages, natural killer cells (NK), antigen-specific cytotoxic T-lymphocytes, and the release of various cytokines in response to an antigen. In more general terms, cellular immunity is not based on antibodies, but on the activation of cells of the immune system. Typically, a cellular immune response may be characterized e.g. by activating antigen-specific cytotoxic T-lymphocytes that are able to induce apoptosis in cells, e.g. specific immune cells like dendritic cells or other cells, displaying epitopes of foreign antigens on their surface.

UTR: The term “untranslated region” or “UTR” or “UTR element” will be recognized and understood by the person of ordinary skill in the art, and are e.g. intended to refer to a part of a nucleic acid molecule typically located 5′ or 3′ located of a coding sequence. An UTR is not translated into protein. An UTR may be part of a nucleic acid, e.g. a DNA or an RNA. An UTR may comprise elements for controlling gene expression, also called regulatory elements. Such regulatory elements may be, e.g., ribosomal binding sites, miRNA binding sites etc.

3′-UTR: The term “3′-untranslated region” or “3′-UTR” or “3′-UTR element” will be recognized and understood by the person of ordinary skill in the art, and are e.g. intended to refer to a part of a nucleic acid molecule located 3′ (i.e. downstream) of a coding sequence and which is not translated into protein. A 3′-UTR may be part of an RNA, located between a coding sequence and an (optional) poly(A) sequence. A 3′-UTR may comprise elements for controlling gene expression, also called regulatory elements. Such regulatory elements may be, e.g., ribosomal binding sites, miRNA binding sites etc.

5′-UTR: The term “5′-untranslated region” or “5′-UTR” or “5′-UTR element” will be recognized and understood by the person of ordinary skill in the art, and are e.g. intended to refer to a part of a nucleic acid molecule located 5′ (i.e. upstream) of a coding sequence and which is not translated into protein. A 5′-UTR may be part of an RNA, located between a coding sequence and an (optional) 5′ cap. A 5′-UTR may comprise elements for controlling gene expression, also called regulatory elements. Such regulatory elements may be, e.g., ribosomal binding sites, miRNA binding sites etc.

Variant (of a sequence): The term “variant” as used throughout the present specification in the context of a nucleic acid sequence will be recognized and understood by the person of ordinary skill in the art, and is e.g. intended to refer to a variant of a nucleic acid sequence derived from another nucleic acid sequence. E.g., a variant of a nucleic acid sequence may exhibit one or more nucleotide deletions, insertions, additions and/or substitutions compared to the nucleic acid sequence from which the variant is derived. A variant of a nucleic acid sequence may at least 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, 99.5% identical to the nucleic acid sequence the variant is derived from. The variant is a functional variant in the sense that the variant has retained at least 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, 99.5% or more of the function of the sequence where it is derived from. In one embodiment a “variant” of a nucleic acid sequence may have at least 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, 99.5% nucleotide identity over a stretch of at least 10, 20, 30, 50, 75 or 100 nucleotide of such nucleic acid sequence.

The term “variant” as used throughout the present specification in the context of polypeptides, proteins or peptides is e.g. intended to refer to a polypeptide, proteins or peptide variant having an amino acid sequence which differs from the original sequence in one or more mutation(s)/substitution(s), such as one or more substituted, inserted and/or deleted amino acid(s). For example, in some aspects an insertion in a protein sequence comprises an insertion of 1 to 10 amino acids, such 1, 2, 3, 4, 5, 6, 7 8, 9 or 10 consecutive amino acids. These fragments and/or variants may have the same, or a comparable specific antigenic property (immunogenic variants, antigenic variants). Insertions and substitutions are possible, in particular, at those sequence positions which cause no modification to the three-dimensional structure or do not affect the binding region. Modifications to a three-dimensional structure by insertion(s) or deletion(s) can easily be determined e.g. using CD spectra (circular dichroism spectra). A “variant” of a protein or peptide may have at least 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, 99.5% amino acid identity over a stretch of at least 10, 20, 30, 50, 75 or 100 amino acids or over the entire length of such protein or peptide. A variant of a protein may comprise a functional variant of the protein, which means, in the context of the invention, that the variant exerts essentially the same, or at least 40%, 50%, 60%, 70%, 75%, 80%, 85%, 90%, 95%, 98% or more of the immunogenicity as the protein it is derived from.

### EXAMPLES

### Example 1—LNP Details and Mouse Immunisation

The LNPs used in the examples herein were ‘RV39’ lipid nanoparticles (composed of 40% cationic lipid LKY750, 10% zwitterionic lipid DSPC, 48% cholesterol, and 2% PEGylated lipid DMG (w/w)). These LNPs were used to produce LNP-formulated recombinant self-amplifying mRNA (SAM) replicons, encoding the HA stem from various influenza strains stabilized on a bacteria ferritin from H. pylori (monodisplay). The HA stem-ferritin fusion gene was generated by fusing the ectodomain of HA to H. pylori ferritin with a Ser-Gly-Gly linker or to either the light or the heavy chain of insect ferritin with a Gly-Gly-Ser-Gly-Gly linker.

### Study A

The immunogenicity of a stem HA H1 candidate vaccine was evaluated in CB6F1 mice. Ten female CB6F1 mice were immunized at days 0 and 28 with:
(a) SAM-stem H1 A/Michigan/45/2015 (a SAM encoding the stem HA H1 A/Michigan/45/2015 polypeptide and H. pylori ferritin (SEQ ID NO: 7)) comprised within LNPs,
(b) QIV (commercially available quadrivalent influenza vaccine comprising inactivated split influenza virions of the strains A/Brisbane/02/2018 H1N1pdm09, A/Kansas/14/2017 H3N2, B/Colorado/06/2017 (B/Victoria) and B/Phuket/3073/2013 (B/Yamagata)) without adjuvant,
(c) QIV formulated with AS03, or
(d) NaCl solution.

Serum samples were collected and analysed as described in examples 3 to 7 below using the assay protocols described in example 2.

Non-inferiority can be concluded if the lower limit (LL) of the 90% Cl for the ratio of the GMTs (GMR) between the compared groups is ≥0.5. Biological/clinical significance (non-inferiority margin) can be concluded if the GMR+90% Cl is >0.5. Statistical superiority can be concluded if the GMR+90% Cl is ≥2.

### Study B

A further subsequent study, analogous to Study A above, was conducted to investigate the impact of administering different doses of SAM encoded stem HA and SAM encoded stem HA polypeptide derived from different strains of influenza. Female CB6F1 mice were immunized with:
(a) SAM-stem H1 A/Michigan/45/2015 (a SAM encoding the stem HA H1 A/Michigan/45/2015 polypeptide and H. pylori ferritin (SEQ ID NO: 7)) comprised within LNPs,
(b) SAM-stem H1 A/New Caledonia/20/99 (a SAM encoding the stem HA H1 A/New Caledonia/20/99 polypeptide and H. pylori ferritin (SEQ ID NO: 6)) comprised within LNPs,
(c) SAM-stem H10 A/Jiangxi-Donghu/346/2013 (a SAM encoding the stem HA H10 A/Jiangxi-Donghu/346/2013 polypeptide and H. pylori ferritin (SEQ ID NO: 9)) comprised within LNPs,
(d) QIV without adjuvant,
(e) QIV formulated with 25 uL AS03, or
(f) NaCl solution.

Fourteen mice were included per groups (a)-(e) and four mice were included in group (f). Serum samples were collected and analysed as described in examples 3 to 7 below using the assay protocols described in example 2.

Non-inferiority can be concluded if the lower limit (LL) of the 90% Cl for the ratio of the GMTs (GMR) between the compared groups is ≥0.5. Biological/clinical significance (non-inferiority margin) can be concluded if the GMR+90% Cl is >0.5. Statistical superiority can be concluded if the GMR+90% Cl is ≥2.

### Study C

Another study was conducted to investigate the heterotypic expression of stem HA from H1 and/or H10 with insect ferritin and assess non-inferiority for the heterotypic (insect) compared to the homotypic (H. pylori) constructs.

In addition, the immunogenicity of different dual (co-mixed) or combo (co-formulated) (stem HA from H1 and H10) candidate vaccines were evaluated in CB6F1 mice. Female CB6F1 mice were immunized at days 0 and 28 with:
(a) SAM encoding the stem HA H1 A/Michigan/45/2015 25 polypeptide and H. pylori ferritin (SEQ ID NO: 7) comprised within LNPs,
(b) SAM encoding the stem HA H10 A/Jiangxi Donghu/346/2013 polypeptide and H. pylori ferritin (SEQ ID NO: 9) comprised within LNPs,
(c) SAM encoding the stem HA H1 A/Michigan/45/2015 25 polypeptide, the stem HA H10 A/Jiangxi Donghu/346/2013 polypeptide and insect ferritin (SEQ ID NO: 15) comprised within LNPs,
(d) SAM encoding the stem HA H1 A/Michigan/45/2015 25 polypeptide and H. pylori ferritin (SEQ ID NO: 7) comprised within LNPs mixed with SAM encoding the stem HA H10 A/Jiangxi Donghu/346/2013 polypeptide and H. pylori ferritin (SEQ ID NO: 9) comprised within LNPs,
(e) SAM encoding the stem HA H1 A/Michigan/45/2015 25 polypeptide and H. pylori ferritin (SEQ ID NO: 7) comprised within LNPs co-formulated with SAM encoding the stem HA H10 A/Jiangxi Donghu/346/2013 polypeptide and H. pylori ferritin (SEQ ID NO: 9) comprised within LNPs,
(f) QIV (commercially available quadrivalent influenza vaccine comprising inactivated split influenza virions of the strains A/Brisbane/02/2018 H1N1pdm09, A/Kansas/14/2017 H3N2, B/Colorado/06/2017 (B/Victoria) and B/Phuket/3073/2013 (B/Yamagata) without adjuvant,
(g) QIV formulated with AS03, or
(h) NaCl solution.

Ten mice were included per groups (a)-(e), eight mice were included per groups (f and g) and four mice were included in group (h). Serum samples were collected and analyzed as described in results section below using the assay protocols described in example 3 to 7 using the assay protocols described in example 2.

Non-inferiority can be concluded if the lower limit (LL) of the 90% Cl for the ratio of the GMTs (GMR) between the compared groups is >0.5. Statistical superiority can be concluded if the LL of the 90% Cl for the ratio of the GMTs (GMR) between the compared groups is >1.

### Example 2—Assay Protocols

### Anti-HA IgG Antibodies by ELISA

Quantification of mouse anti-HA IgG antibodies was performed by ELISA using HA antigen (full length or stem only) as coating diluted at a concentration of 4 μg/ml in PBS (50 μl/well). The plates were then incubated for 1 hour at 37° C. in saturation buffer. Diluted sera were added to the coated plates (50 μl/well) and incubated for 90 minutes at 37° C. The plates were washed prior to the adding of diluted peroxydase conjugated goat anti-mouse IgG. The reaction was stopped with H2SO4 2N and optical densities were read at 490-620 nm. The titers were expressed as ELISA Units Titers (EU/ml).

### Stem Specific T Cell Frequencies

Spleens were collected and placed in complemented RPMI Cell suspensions were prepared from each spleen using a tissue grinder. The splenic cell suspensions were filtered, harvested, centrifuged and resuspended in Complete Medium. Fresh splenocytes were then plated in 96-well plates in presence of overlapping peptide pool covering the sequence of H1 Mich 15 stem. Following stimulation, cells were stained and analyzed using a 5-colour ICS assay. Cells were washed and stained with anti-CD16/32, anti-CD4-V450 and anti-CD8-PerCp-Cy5.5 antibodies. Live/dead-PO was added for 30 min at 4° C. Cells were permeabilized and stained with anti-IL2-FITC, anti-IFNγ-APC and anti-TNFα-PE antibodies. Stained cells were analyzed by flow cytometry using a LSRII and the FlowJo software.

### Neutralization Antibody Titers

Quantification of mouse neutralizing antibody titers was assessed by microneutralization assay. Briefly, mouse sera were diluted and incubated in presence of reporter influenza virus. After incubation, the serum-virus mix were added on cell culture. Influenza-positive cells were analysed and quantified by flow cytometry. Titers are expressed as 50% neutralization titers (1050), corresponding to reduction titers calculated by regression analysis of the inverse dilution of serum that provided 50% cell infected reduction compared to control wells (virus only, no serum).

### Example 3—Anti-H1 Stem IgG Antibody Titers by ELISA at 14 Days Post Dose 2

IgG antibody titers directed towards H1-stem were measured by ELISA assay at 14 days post second immunization (day 42).

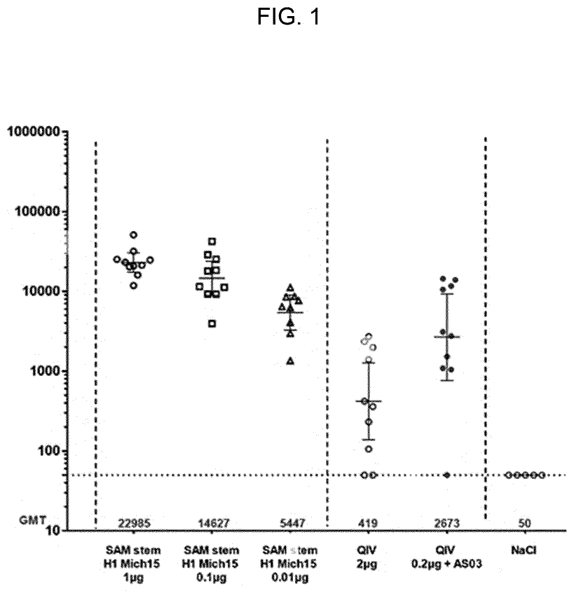

The results from Study A are shown in FIG. 1 High anti-H1 stem IgG antibodies were induced by SAM H1 stem, comparable to and even improved (1 μg) compared to titers induced by QIV/AS03 immunisation (SAM stem H1 1 μg/QIV: GMR 54.83 and LL 21.94; SAM stem H1 1 μg/QIV+AS03: GMR 8.60 and LL 3.08). ELISA titers are expressed as midpoint values (Geomean with 95% Cl).

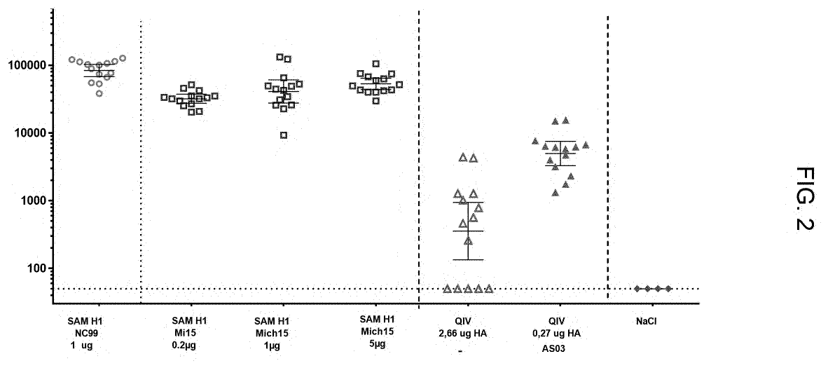

The results from Study B are shown in FIG. 2. High anti-H1 stem IgG antibodies were induced by SAM stem H1/NC/99, comparable to and even improved compared to titers induced by QIV/AS03 immunisation (SAM stem H1/NC/99/QIV: GMR 235.88 and LL 100.78; SAM stem H1/NC/99/QIV+AS03: GMR 16.86 and LL 12.34). High anti-H1 stem IgG antibodies were induced by SAM stem H1/Mich/15, comparable to and even improved (0.2 μg, 1 μg and 5 μg) compared to titers induced by QIV/AS03 immunisation (SAM stem H1/Mich/15 0.2 μg/QIV: GMR 90.17 and LL 38.79; SAM stem H1/Mich/15 0.2 μg/QIV+AS03: GMR 6.45 and LL 4.83).

ELISA titers are expressed as 50% endpoint titers (individual animals with GMT and IC95).

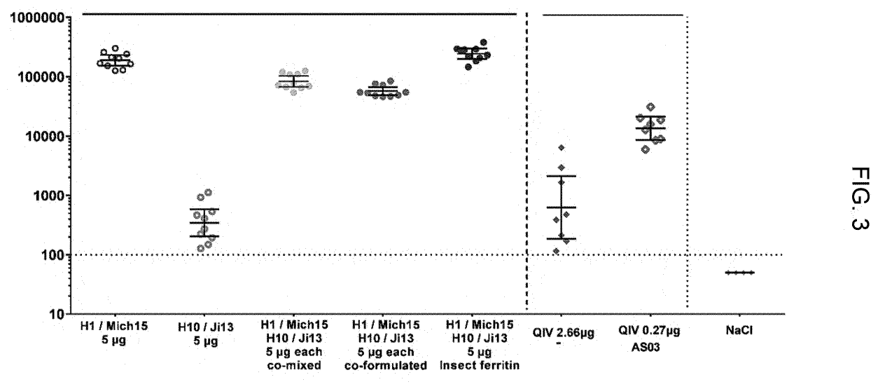

The results from Study C are shown in FIG. 3. ELISA titers are expressed as endpoint values (individual values with GMT and 95% Cl). Anti-H1 stem IgG antibody titers were measured by using the conserved HA stem region from A/H1N1/New Caledonia/20/1999 as coating antigen. The non-inferiority of SAM H1/H10 stem heterotypic construct (insect ferritin) over SAM H1/Mich15 homotypic construct (H. pylori ferritin) was shown (LL of 90% Cl of GMR above 0.5-fold), and even improved compared to SAM H1/Mich15 homotypic construct (H. pylori ferritin) co-mixed with SAM H10/Ji/13 homotypic construct (H. pylori ferritin) (GMR 2.93 and LL 2.39).

The dotted horizontal line on the figures corresponds to the threshold of detection.

### Example 4—Anti-H1/NC/99 and Anti-H1/Mich/15 IgG Antibody Titers by ELISA at 14 Days Post Dose 2

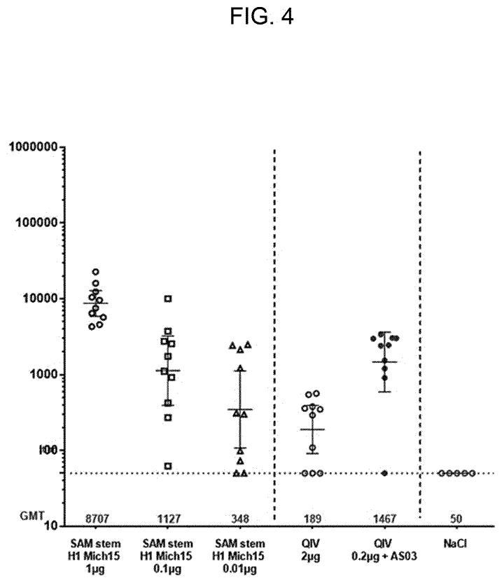

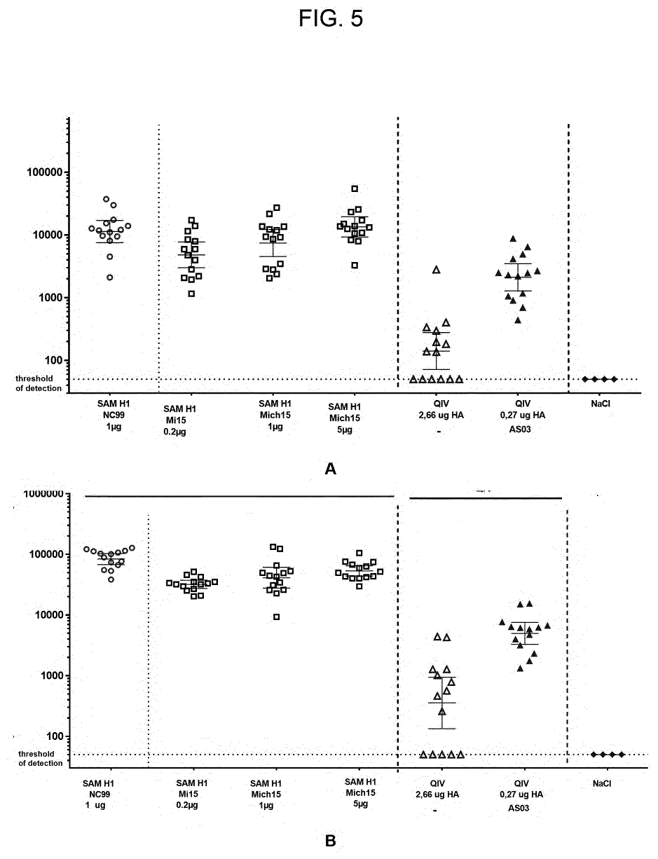

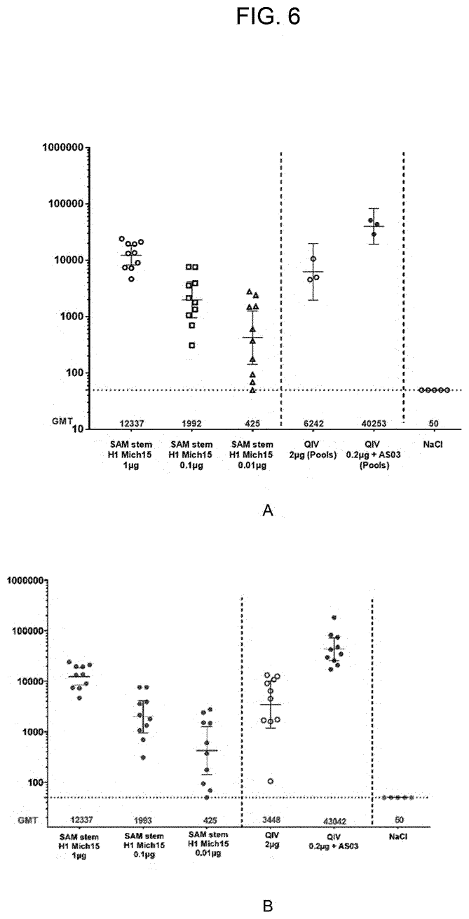

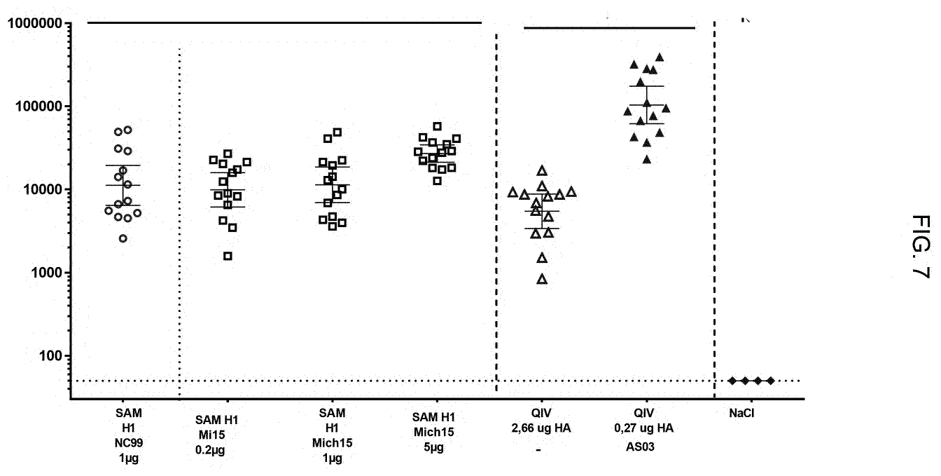

IgG antibody titers directed towards H1 were measured by ELISA assay using a full-length (trimeric protein with foldon and without transmembrane domain) A/H1N1/New Caledonia/20/1999 polypeptide (Study A, FIG. 4 and Study B, FIG. 5A) or a full-length A/H1N1/Michigan/2015 polypeptide (Study A, FIGS. 6A and 6B and Study B, FIG. 7) at 14 days post second immunization (day 42).

Study A has revealed that high anti-H1 NC99 IgG antibodies were induced by SAM H1 stem, improved (1 μg) compared to titers induced by QIV/AS03 immunisation (SAM stem H1 1 μg/QIV: GMR 46.10 and LL 21.00; SAM stem H1 1 μg/QIV+AS03: GMR 5.94 and LL 2.30). High anti-H1 Mich15 IgG antibodies were induced by SAM H1 stem, improved (1 μg) compared to titers induced by QIV immunisation (SAM stem H1/QIV: GMR 3.58 and LL 1.19).

Study B has revealed that high anti-H1/NC/99 IgG antibodies were induced by SAM stem H1/NC/99 and H1/Mich/15 (0.2 μg, 1 μg and 5 μg), and even improved compared to titers induced by QIV/AS03 immunisation (SAM H1/NC/99/QIV: GMR 80.64 and LL 42.74; SAM H1/NC/99/QIV+AS03: GMR 5.37 and LL 3.19; SAM H1/Mich/15 0.2 μg/QIV: GMR 34.20 and LL 17.60; SAM H1/Mich/15 0.2 μg/QIV+AS03: GMR 2.28 and LL 1.30). High anti-H1/Mich/15 IgG antibodies were induced by SAM stem H1/NC/99 and H1/Mich/15 (1 μg and 5 μg), improved compared to titers induced by QIV immunisation (SAM H1/NC/99/QIV: GMR 2.05 and LL 1.12; SAM H1/Mich/15 1 μg/QIV: GMR 2.08 and LL 1.19).

In Study B only, the experiment was repeated using a stem-only A/H1N1/New Caledonia/20/1999 polypeptide as coating antigen. The results are shown in FIG. 5B.

For FIG. 4 and FIGS. 6A et 6B, ELISA titers are expressed as midpoint values (Geomean with 95% Cl). For FIGS. 5A and 5B and FIG. 7, ELISA titers are expressed as 50% endpoint titers (individual animals with GMT and IC95).

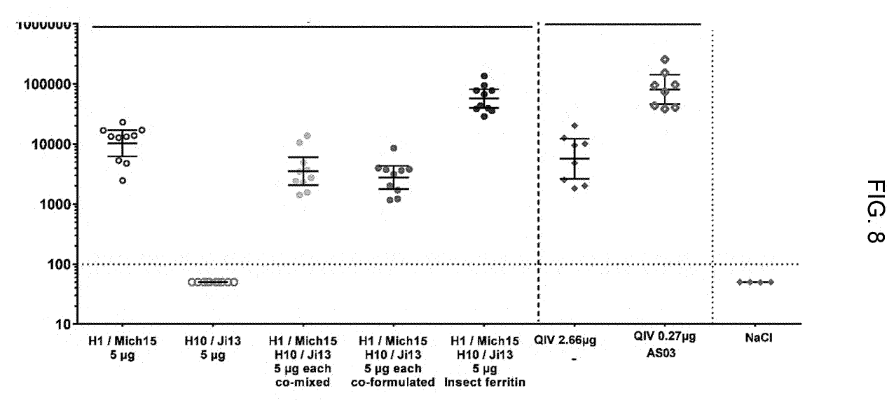

The results from Study C are shown in FIG. 8. IgG antibody titers directed towards H1 were measured by ELISA assay using a full-length (trimeric protein with foldon and without transmembrane domain) A/H1N1/Michigan/2015 polypeptide at 14 days post second immunization (day 42). ELISA titers are expressed as endpoint values (individual values with GMT and 95% Cl). The non-inferiority of SAM H1/H10 stem heterotypic construct (insect ferritin) over SAM H1/Mich15 homotypic construct (H. pylori ferritin) was shown (LL of 90% Cl of GMR above 0.5-fold). The data even suggest that this heterotypic construct induced higher responses compared to SAM H1/Mich15 homotypic construct (H. pylori ferritin): increase is closed to 5-fold (estimated GMR of 5.55 with 90% Cl from 3.54-fold to 8.72-fold). The data further show that this heterotypic construct induced higher response compared to SAM H1/Mich15 homotypic construct (H. pylori ferritin) co-mixed with SAM H1/Mich15 homotypic construct (H. pylori ferritin): GMR of 16.13 and LL of 9.79.

The dotted horizontal line on the figures corresponds to the threshold of detection.

### Example 5—Anti-Group A1 (H2, H9, H18) IgG Antibody Titers by ELISA at 14 Days Post Dose 2

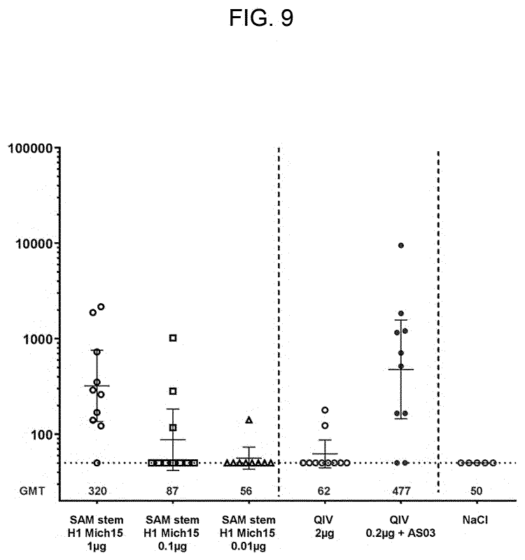

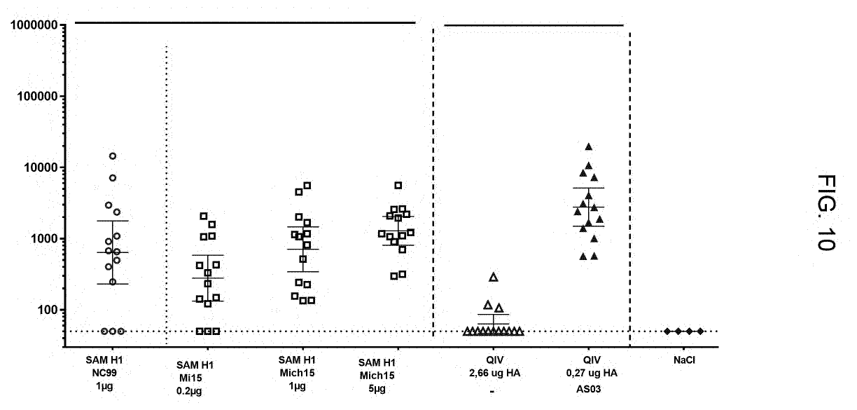

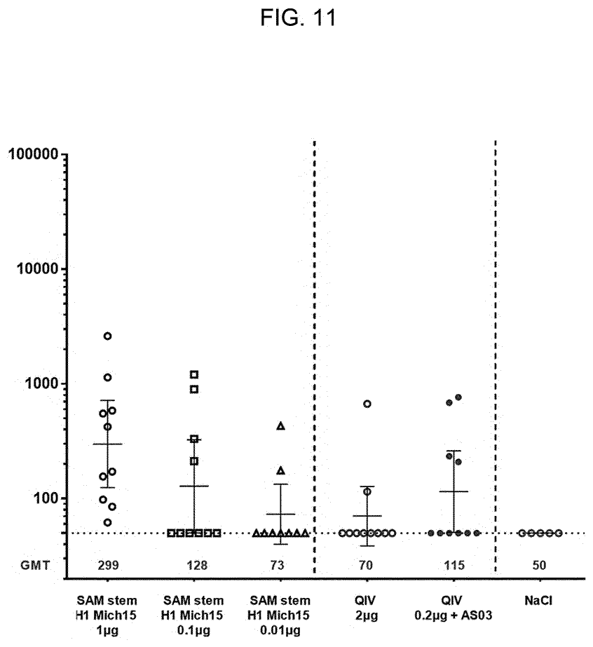

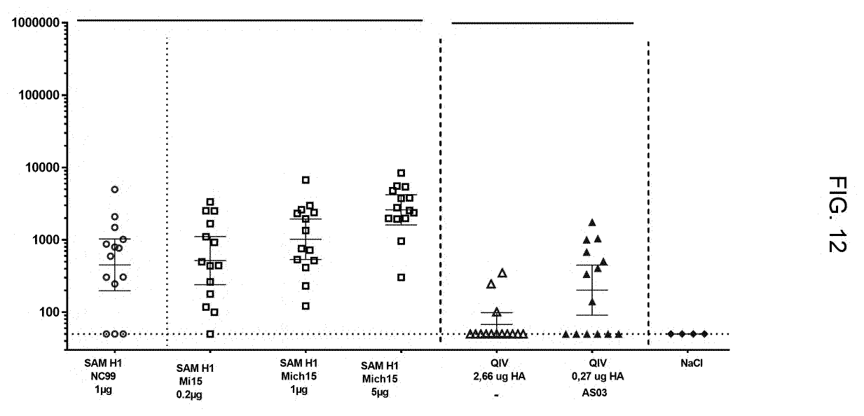

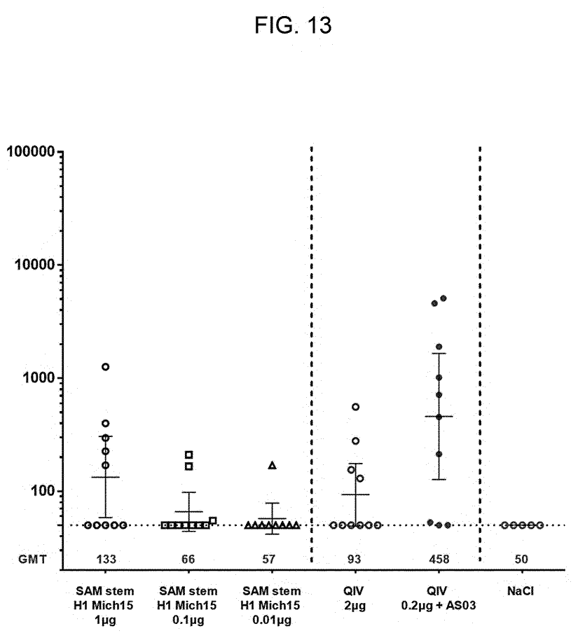

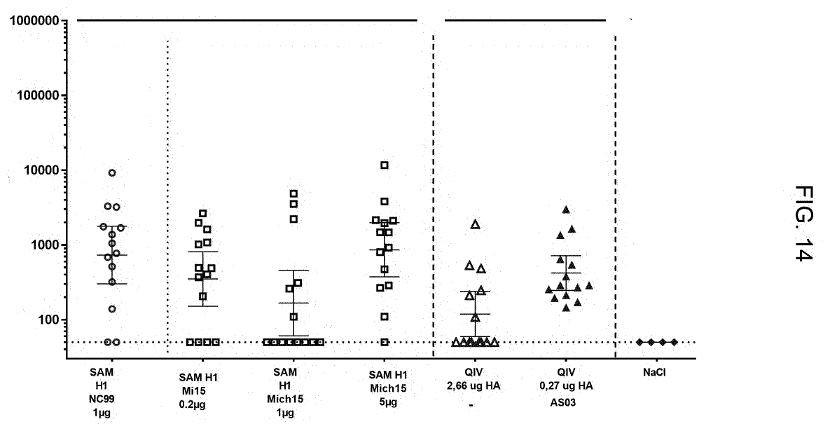

IgG antibody titers directed towards group A1 HA were measured by ELISA assay using a full-length H2 (Study A, FIG. 9 and Study B, FIG. 10), a full-length H9 (Study A, FIG. 11 and Study B, FIG. 12) or a full-length H18 (Study A, FIG. 13 and Study B, FIG. 14) at 14 days post second immunization (day 42).

Study A has revealed that anti-H2, anti-H9 and anti-H18 IgG antibodies are induced by SAM-stem H1.

Study B has revealed that anti-H2 IgG antibodies are induced by SAM-stem H1/NC/99 and H1/Mich/15 (0.2 μg, 1 μg and 5 μg), and even improved compared to titers induced by QIV immunisation (SAM H1/NC/99/QIV: GMR 10.07 and LL 4.09; SAM H1/Mich/15 0.2 μg/QIV: GMR 4.38 and LL 2.25).

Study B has further revealed that anti-H9 IgG antibodies are induced by SAM-stem H1/NC/99 and H1/Mich/15 (0.2 μg, 1 μg and 5 μg), and even improved compared to titers induced by QIV/AS03 immunisation (SAM H1/NC/99/QIV: GMR 6.66 and LL 3.11; SAM H1/Mich/15 0.2 μg/QIV: GMR 7.63 and LL 3.74; SAM H1/NC/99/QIV+AS03: GMR 2.23 and LL 0.89; SAM H1/Mich/15 0.2 μg/QIV+AS03: GMR 2.55 and LL 1.06).

Study B has further revealed that anti-H18 IgG antibodies are induced by SAM-stem H1/NC/99 and H1/Mich/15 (0.2 μg, 1 μg and 5 μg), and even improved compared to titers induced by QIV immunisation (SAM H1/NC/99/QIV: GMR 6.17 and LL 2.62; SAM H1/Mich/15 0.2 μg/QIV: GMR 2.96 and LL 1.26).

For FIG. 9, FIG. 11 and FIG. 13, ELISA titers are expressed as midpoint values (Geomean with 95% Cl). For FIG. 10, FIG. 12 and FIG. 14, ELISA titers are expressed as 50% endpoint titers (individual animals with GMT and 1095).

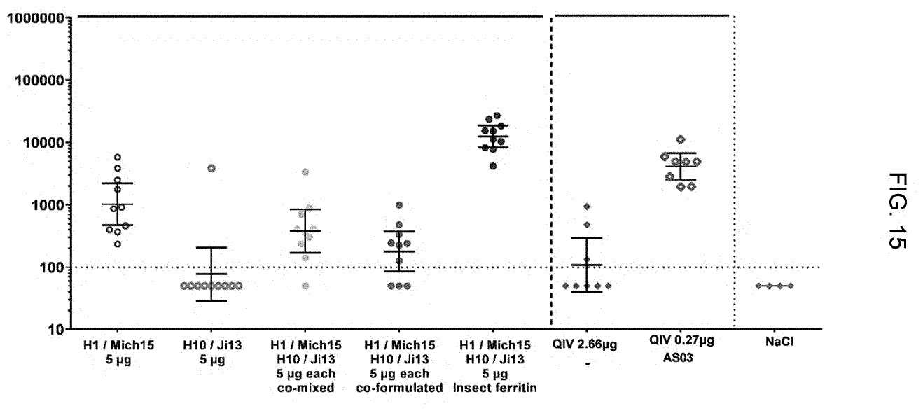

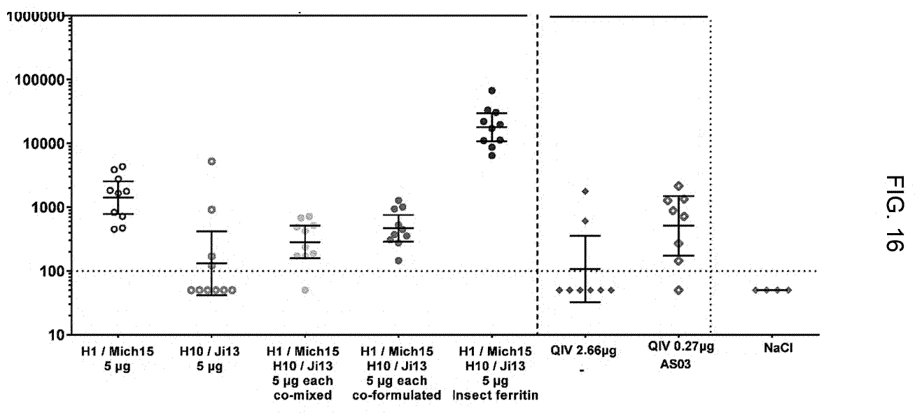

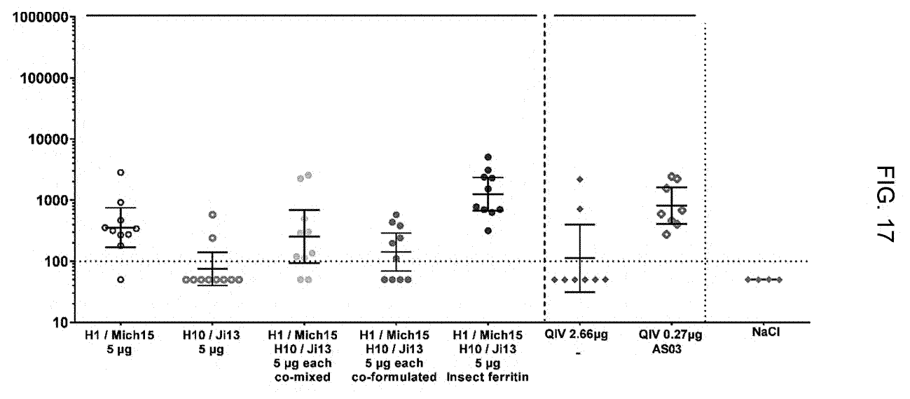

The results from Study C are shown in FIGS. 15, 16 and 17. IgG antibody titers directed towards group A1 HA were measured by ELISA assay using a full-length H2 (FIG. 15), a full-length H9 (FIG. 16) or a full-length H18 (FIG. 17) at 14 days post second immunization (day 42). ELISA titers are expressed as endpoint values (individual values with GMT and 95% Cl). The non-inferiority of SAM H1/H10 stem heterotypic construct (insect ferritin) over SAM H1/Mich15 homotypic construct (H. pylori ferritin) was shown (LL of 90% Cl of GMR above 0.5-fold). High anti-H2, anti-H9 and anti-H18 IgG antibodies are induced by SAM H1/H10 insect ferritin. The data even suggest that this heterotypic construct induced higher responses compared to SAM H1/Mich15 homotypic construct (H. pylori ferritin): anti-H2-GMR of 12.16 and LL of 7.15; anti-H9-GMR of 12.78 and LL of 7.03; anti-H18-GMR of 3.55 and LL of 1.76. The data further show that this heterotypic construct induced higher response compared to SAM H1/Mich15 homotypic construct (H. pylori ferritin) co-mixed with SAM H1/Mich15 homotypic construct (H. pylori ferritin): anti-H2-GMR of 32.79 and LL of 16.78; anti-H9-GMR of 63.61 and LL of 36.01; anti-H18-GMR of 4.95 and LL of 2.08.

The dotted horizontal line on the figures corresponds to the threshold of detection.

### Example 6—Anti-Group A2 (H3, H7, H10) IgG Antibody Titers by ELISA at 14 Days Post Dose 2

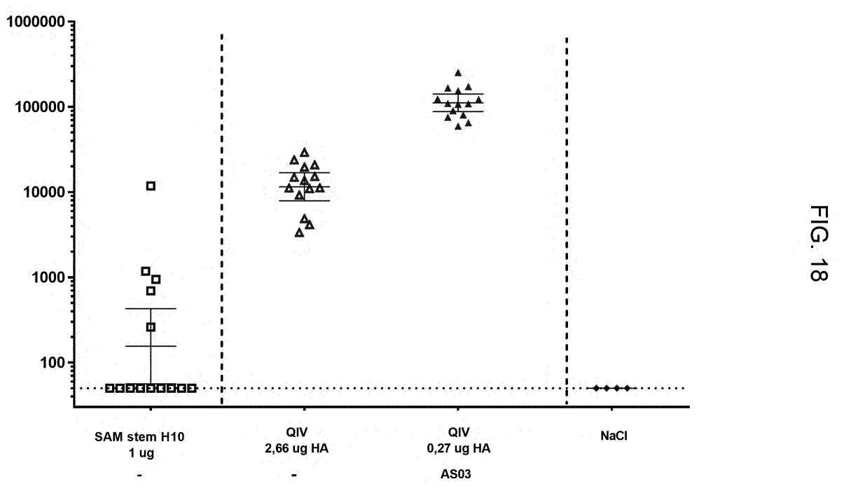

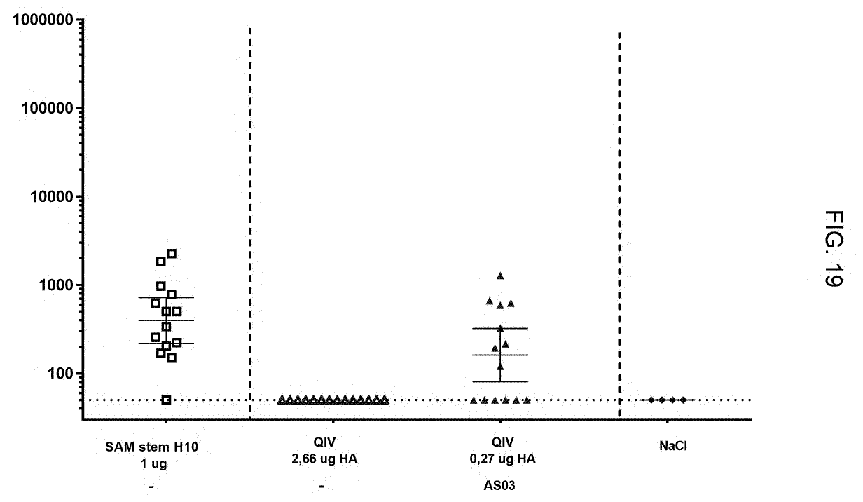

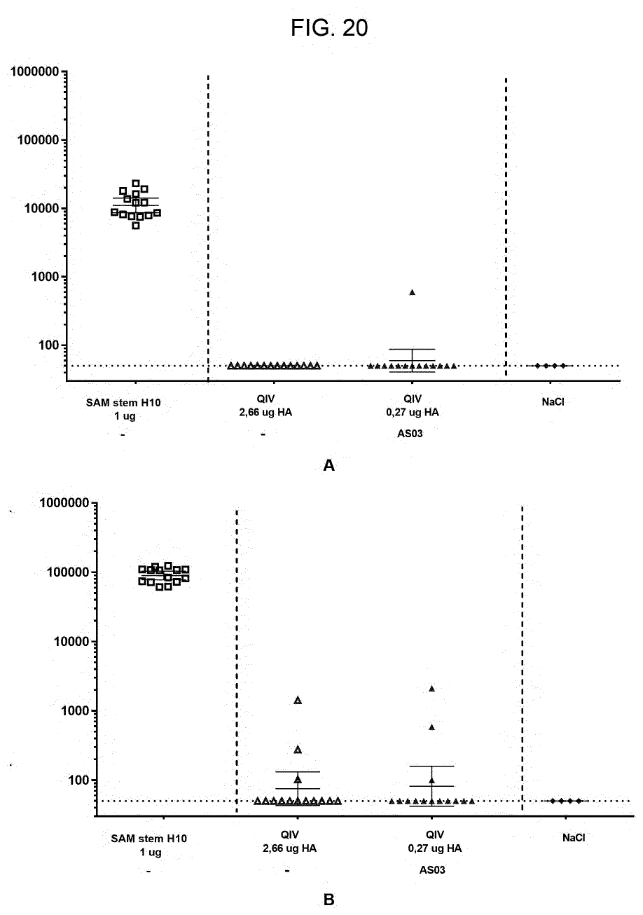

This experiment was carried out for Study B. IgG antibody titers directed towards group A2 HA were measured by ELISA assay using a full-length H3 protein (FIG. 18), a full-length H7 protein (FIG. 19) or a full length H10 protein (FIG. 20A) at 14 days post second immunization (day 42).

Study B has revealed that anti-H3 and anti-H10 IgG antibodies are induced by SAM-stem H10/Ji/13.

Study B has further revealed that anti-H7 IgG antibodies are induced by SAM-stem H10/Ji/13, and even improved compared to titers induced by QIV/AS03 immunisation (SAM H10/Ji/13/QIV+AS03: GMR 2.46 and LL 1.16).

The H10 ELISA experiment was repeated using a stem-only polypeptide as coating antigen. The results are shown in FIG. 20B.

ELISA titers are expressed as 50% endpoint titers (individual animals with GMT and IC95).

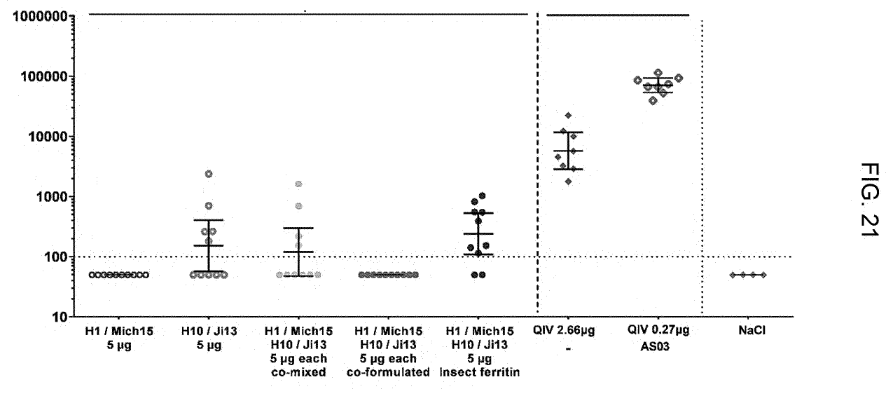

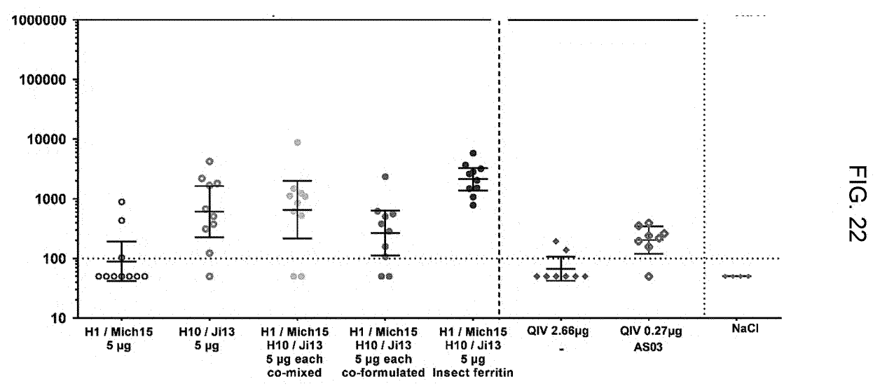

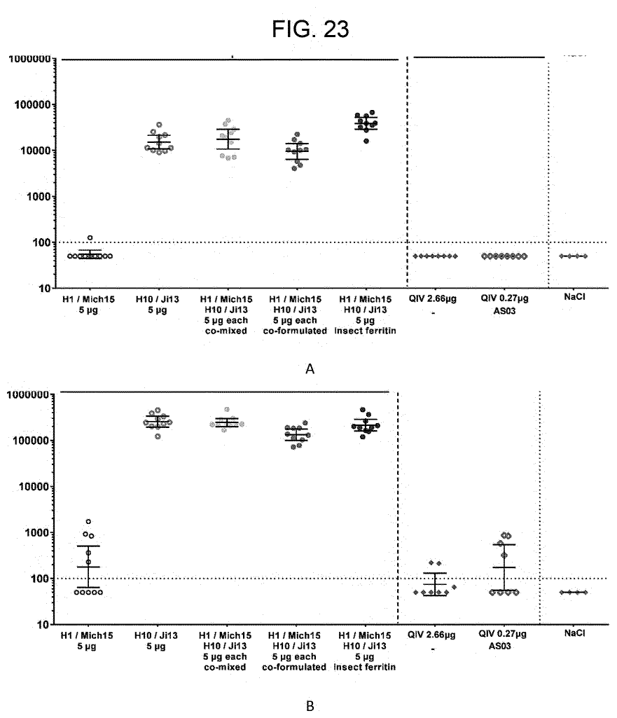

This experiment was further carried out for Study C. IgG antibody titers directed towards group A2 HA were measured by ELISA assay using a full-length H3 protein (FIG. 21), a full-length H7 protein (FIG. 22) or a full length H10 protein (FIG. 23A) at 14 days post second immunization (day 42). The H10 ELISA experiment was repeated using a stem-only polypeptide as coating antigen (FIG. 23B). ELISA titers are expressed as endpoint values (individual values with GMT and 95% Cl). The non-inferiority of the SAM H1/H10 stem heterotypic construct (insect ferritin) over H1/Mich15 homotypic construct (H. pylori ferritin) was shown (LL of 90% Cl of GMR above 0.5-fold). High anti-H3, anti-H7 and anti-H10 IgG antibodies were induced by SAM H1/H10 insect ferritin. The data even suggest that this heterotypic construct induced higher responses compared to SAM H1/Mich15 homotypic construct (H. pylori ferritin): anti-H10-GMR of 2.54 and LL of 1.79; anti-H7-GMR of 3.50 and LL of 1.53. The data further show that this heterotypic construct induced higher response compared to SAM H1/Mich15 homotypic construct (H. pylori ferritin) co-mixed with SAM H1/Mich15 homotypic construct (H. pylori ferritin): anti-H10-GMR of 2.21 and LL of 1.48; anti-H7-GMR of 3.27 and LL of 1.32.

The non-inferiority of the SAM H1/Mich15 homotypic construct (H. pylori ferritin) co-mixed with SAM H10/Jiangxi/13 homotypic construct (H. pylori ferritin) over SAM H10/Jiangxi/13 homotypic construct (H. pylori ferritin) was shown (LL of 90% Cl of GMR above 0.5-fold).

The dotted horizontal line on the figures corresponds to the threshold of detection.

### Example 7—H1/Mich/15 Stem Specific CD4+ and CD8+ T Cells Frequencies at 14 Days Post Dose 2

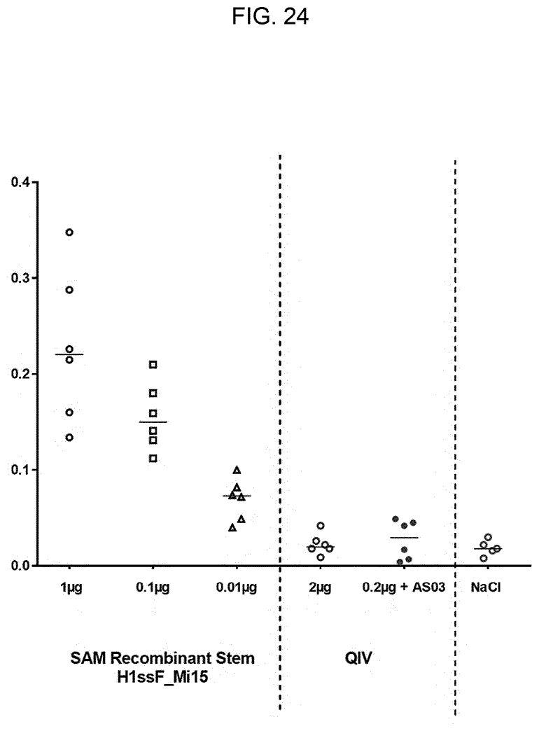

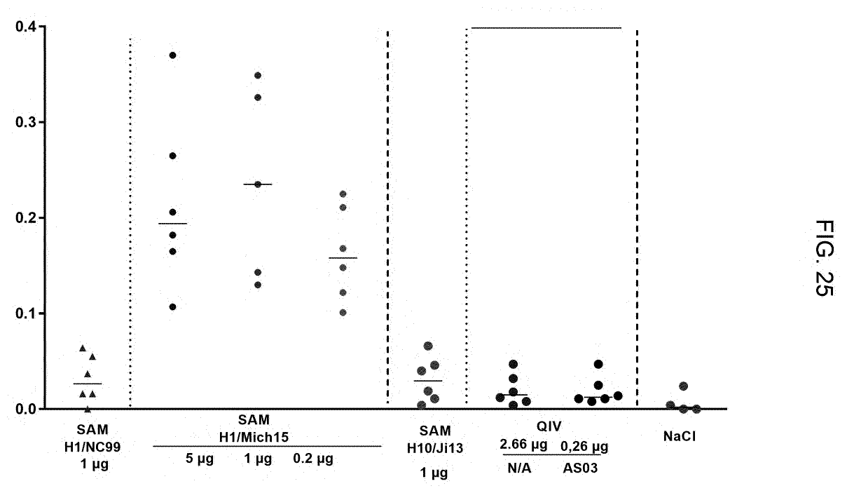

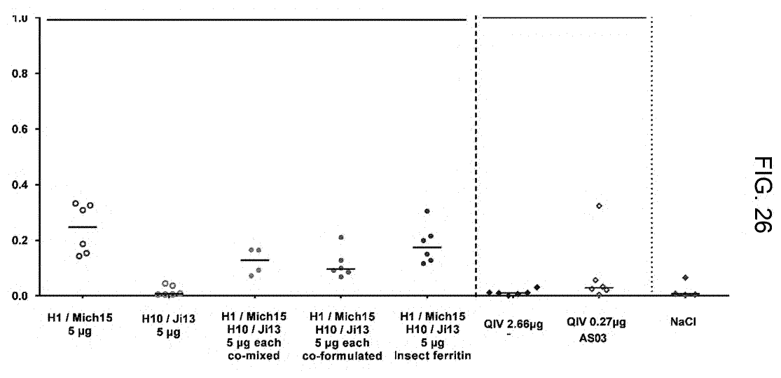

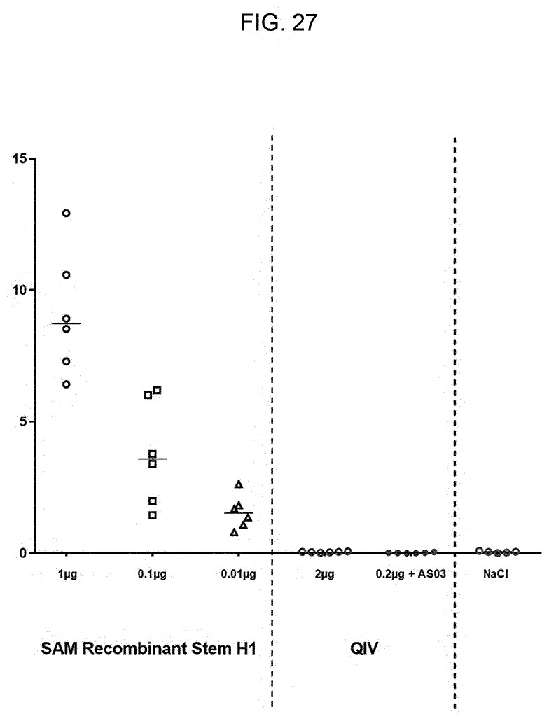

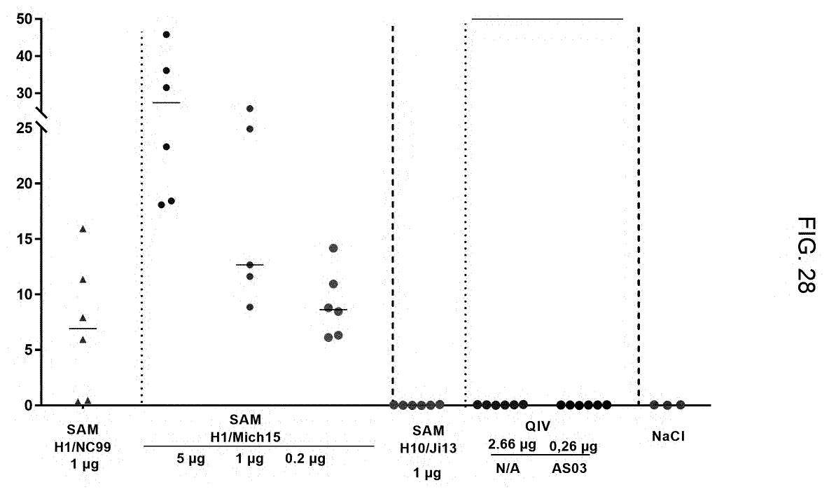

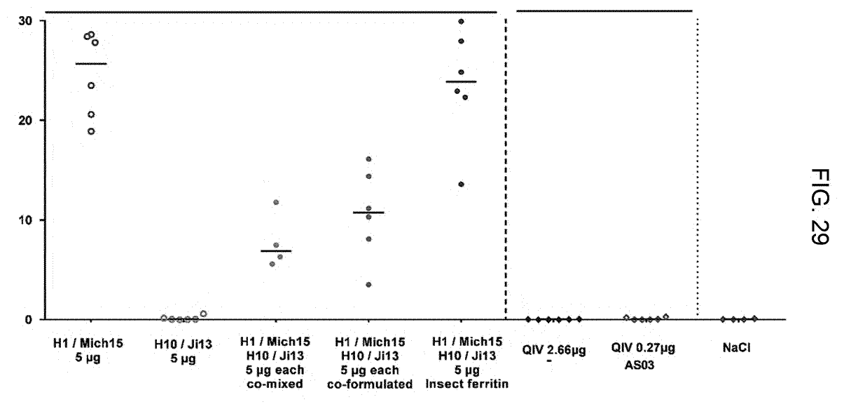

The T cell response induced by the stem H1 candidate vaccine was evaluated. The percentage of H1 stem-specific CD4+ T cells (Study A, FIG. 24; Study B, FIG. 25 and Study C, FIG. 26) and CD8+ T cells (Study A, FIG. 27; Study B, FIG. 28 and Study C, FIG. 29) were measured 14 days after the second immunization. Intracellular staining was performed on splenocytes after a 6 hours re-stimulation with peptide pools covering the sequence of H1 stem (A/Michigan/45/2015).

For all the studies, higher frequencies of H1/mich/15 stem specific CD4+ T cell were observed with the SAM-stem H1 antigen as compared to QIV with or without AS03 (e.g. Study B—SAM H1/Mich/15 0.2 μg/QIV: GMR 10.58 and LL 6.52; SAM H1/Mich/15 0.2 μg/QIV+AS03: GMR 9.85 and LL 6.39).

For FIG. 24, the results are expressed as percentage of H1 A/Michigan/45/2015 stem-specific CD4+ T cells expressing IFNγ and/or IL2 and/or TNFα and/or IL13 and/or IL17 (individual animals with medians).

For FIG. 25, the results are expressed as percentage of stem H1 FLU pool of peptides-specific CD4+ T cells expressing IFNγ and/or IL2 and/or TNFα (individual animals with medians).

For FIG. 26, the results are expressed as percentage of H1 A/Michigan/45/2015 stem-specific CD4+ T cells expressing IFNγ and/or IL2 and/or TNFα (individual animals with medians).

For all the studies, higher frequencies of H1/Mich/15 stem specific CD8+ T cell were observed with the SAM-stem H1 antigen as compared to QIV with or without AS03 (e.g. Study B—SAM H1/NC/99/QIV: GMR 59.82 and LL 19.56; SAM H1/NC/99/QIV+AS03: GMR 106.61 and LL 32.30; H1/Mich/15 0.2 μg/QIV: GMR 158.44 and LL 110.40; SAM H1/Mich/15 0.2 μg/QIV+AS03: GMR 282.38 and LL 134.11). In addition, for Study C, a higher frequency of the SAM H1/H10 insect ferritin construct compared to SAM H1/Mich15 homotypic construct (H. pylori ferritin) co-mixed with SAM H1/Mich15 homotypic construct (H. pylori ferritin) has been observed: GMR of 3.07 and LL of 2.03.

For FIG. 27, the results are expressed as percentage of H1 A/Michigan/45/2015 stem-specific CD8+ T cells expressing IFNγ and/or IL2 and/or TNFα and/or IL13 and/or IL17 (individual animals with medians).

For FIG. 28, the results are expressed as percentage of stem H1 FLU pool of peptides-specific CD8+ T cells expressing IFNγ and/or IL2 and/or TNFα (individual animals with medians).

For FIG. 29, the results are expressed as percentage of H1 A/Michigan/45/2015 stem-specific CD8+ T cells expressing IFNγ and/or IL2 and/or TNFα (individual animals with medians).

The dotted horizontal line on the figures corresponds to the threshold of detection.

### Example 8—H1 0/Jiangxi-Donghu Stem Specific CD4+ and CD8+ T Cells Frequencies at 14 Days Post Dose 2

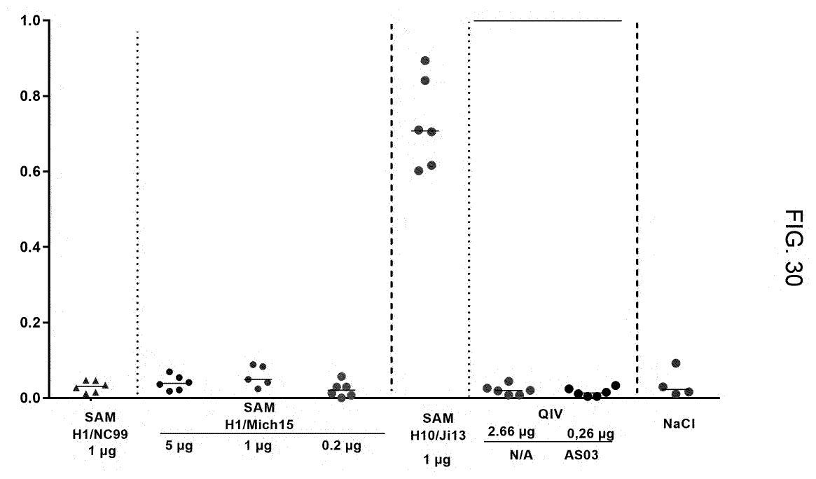

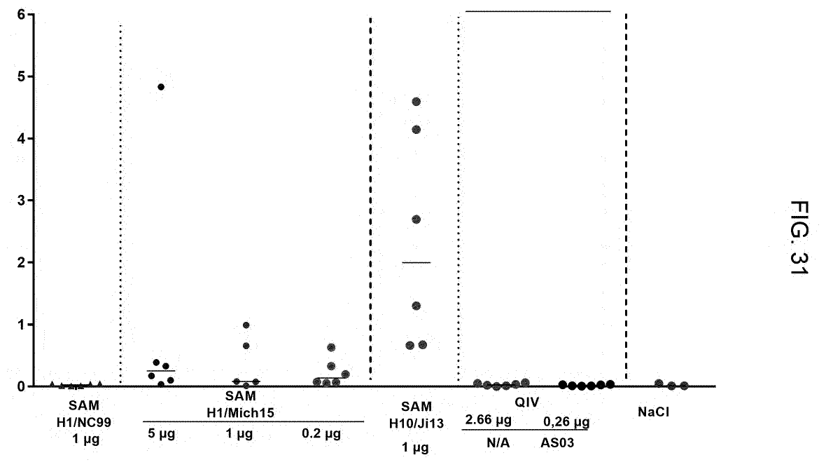

This experiment was carried out for Study B. The percentage of H10 stem-specific CD4+ T cells (FIG. 30) and CD8+ T cells (FIG. 31) were measured 14 days after the second immunization. Intracellular staining was performed on splenocytes after a 6 hours re-stimulation with peptide pools covering the sequence of H10 stem (H10/Jiangxi-Donghu).

Higher frequencies of H1/NC/99, H1/mich/15 (1 μg) and H10/Ji/13 stem specific CD4+ T cell were observed with the SAM-stem H10 antigen as compared to QIV with AS03 (SAM H1/NC/99/QIV+AS03: GMR 2.32 and LL 1.19; SAM H1/Mich/15 1 μg/QIV+AS03: GMR 4.65 and LL 2.23; SAM H10/Ji/13/QIV+AS03: GMR 63.69 and LL 35.53).

Higher frequencies of H1/Mich/15 and H10/Ji/13 stem specific CD8+ T cell were observed with the SAM-stem H10 antigen as compared to QIV with or without AS03 (SAM H10/Ji/13/QIV: GMR 112.08 and LL 23.70; SAM H10/Ji13/QIV+AS03: GMR 101.58 and LL 47.44; H1/Mich/15 0.2 μg/QIV: GMR 9.63 and LL 1.91; SAM H1/Mich/15 0.2 μg/QIV+AS03: GMR 8.72 and LL 3.23).

For FIG. 30, the results are expressed as percentage of stem H10 FLU pool of peptides-specific CD4+ T cells expressing IFNγ and/or IL2 and/or TNFα (individual animals with medians).

For FIG. 31, the results are expressed as percentage of stem H10 FLU pool of peptides-specific CD8+ T cells expressing IFNγ and/or IL2 and/or TNFα (individual animals with medians).

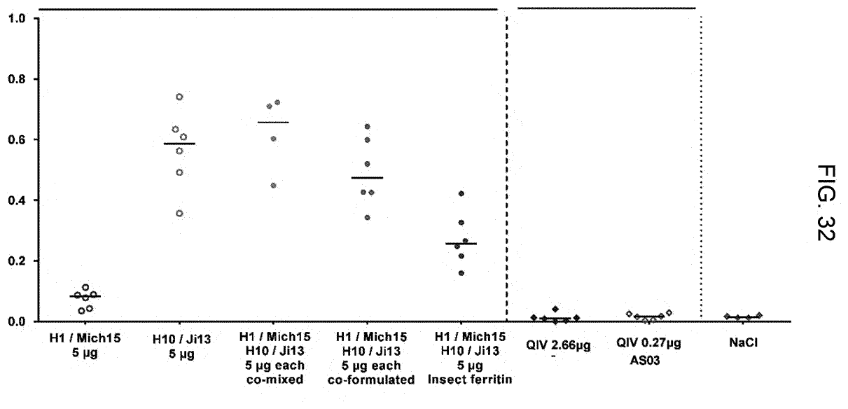

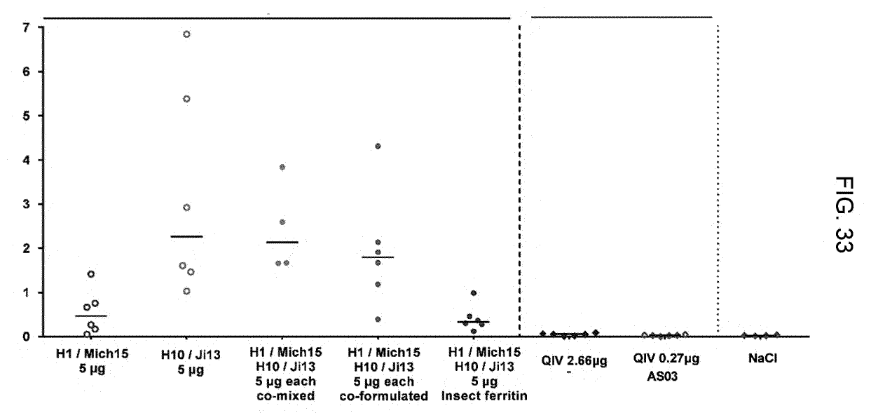

This experiment was further carried out for Study C. The percentage of H10 stem-specific CD4+ T cells (FIG. 32) and CD8+ T cells (FIG. 33) were measured 14 days after the second immunization. Intracellular staining was performed on splenocytes after a 6 hours restimulation with peptide pools covering the sequence of H10 stem (H10/Jiangxi-Donghu). Higher frequencies of H10/Jiangxi/13/2013 stem specific CD4+ and CD8+ T cell were observed with the SAM-stem H10 antigen compared to QIV with or without AS03.

For FIG. 32, the results are expressed as percentage of H10 A/Jiangxi/13/2013 stem-specific CD4+ T cells expressing IFNγ and/or IL2 and/or TNFα (individual animals with medians).

For FIG. 33, the results are expressed as percentage of H10 A/Jiangxi/13/2013 stem-specific CD8+ T cells expressing IFNγ and/or IL2 and/or TNFα (individual animals with medians).

The dotted horizontal line on the figures corresponds to the threshold of detection.

### Example 9—Group A1 H1/Mich/15, H1/NC/99 and H5/Vn/04 Microneutralization Titers at 14 Days Post Dose 2

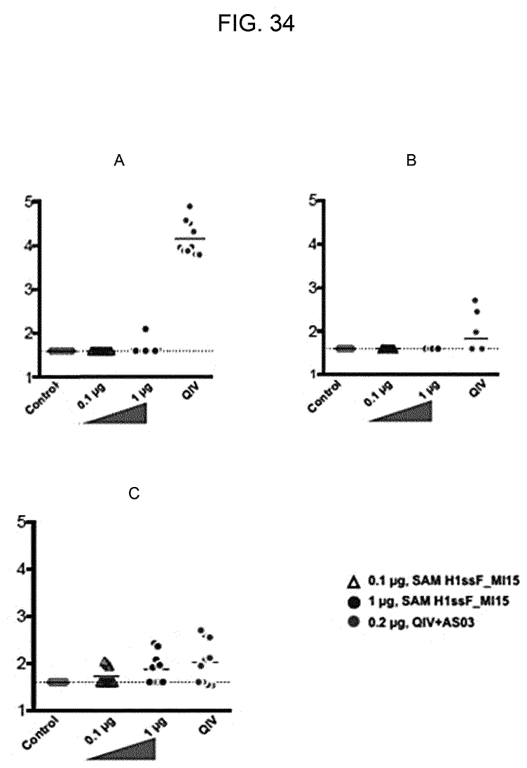

Microneutralization titers towards group A1 influenza virus were measured by microneutralisation assay using H1/Mich/15 (panel A), H1/NC/99 (panel B) or H5/Vn/04 (panel C) reporter viruses (FIG. 34). The results are expressed as 1050 (log₁₀ dilution).

The dotted horizontal line on the figures corresponds to the threshold of detection.
Corbett, et al. Design of Nanoparticulate Group 2 Influenza Virus Hemagglutinin Stem Antigens That Activate Unmutated Ancestor B Cell Receptors of Broadly Neutralizing Antibody Lineages, mBio. 2019 10(1):e02810-18
Corti, et al. A neutralizing antibody selected from plasma cells that binds to group 1 and group 2 influenza A hemagglutinins, Science 2011 333, 850-856
Dayhoff M, O et al. A model of evolutionary change in proteins, Atlas of Protein Sequence and Structure, 1978 vol 5, supp. 3.
Jiang S, et al., Lipidoid-Coated Iron Oxide Nanoparticles for Efficient DNA and siRNA delivery Nano Lett., 13:1059-1064 (2013)
Steel J, et al. Influenza virus vaccine based on the conserved hemagglutinin stalk domain, mBio. 2010 1(1):e00018-10
Zhang, C et al. Advances in mRNA vaccines for infectious diseases, Front. Immunol. 2019, 10:594.

1-94. (canceled)

95. A carrier-formulated mRNA comprising at least one coding sequence encoding an influenza HA stem polypeptide.

96. The carrier-formulated mRNA according to claim 95, wherein the carrier is a lipid nanoparticle (LNP).

97. The carrier-formulated mRNA according to claim 96, wherein the LNP comprise a PEG-modified lipid, a non-cationic lipid, a sterol, and a non-ionisable cationic lipid.

98. The carrier-formulated mRNA according to claim 96, wherein the LNP comprise a PEG-modified lipid, a non-cationic lipid, a sterol, and an ionisable cationic lipid.

99. The carrier-formulated mRNA according to claim 95, wherein the mRNA comprises at least one additional coding sequence which encodes a protein nanoparticle, and wherein the protein nanoparticle is ferritin.

100. The carrier-formulated mRNA according to claim 99, wherein the ferritin is insect ferritin.

101. The carrier-formulated mRNA according to claim 95, wherein the influenza HA stem polypeptide is derived from influenza A Group 1, or influenza A Group 2.

102. The carrier-formulated mRNA according to claim 101, wherein influenza A Group 1 comprises:
(i) subtype H1, H2, H5, H6, H8, H9, H11, H12, H13, H16, H17 or H18; or
(ii) subtype H1.

103. The carrier-formulated mRNA according to claim 101, wherein influenza A Group 2 comprises:
(i) subtype H3, H4, H7, H10, H14 or H15;
(ii) H3 or H10; or
(iii) H10.

104. The carrier-formulated mRNA according to claim 95, wherein the influenza HA stem polypeptide is 130 to 400 residues in length, 160 to 300 in length, 180 to 250 in length, or 190 to 220 in length.

105. The carrier-formulated mRNA according to claim 95, comprising two or more coding sequences each encoding an influenza HA stem polypeptide, wherein the coding sequences are encoded on the same mRNA molecule.

106. The carrier-formulated mRNA according to claim 105, wherein the two or more coding sequences encode different influenza HA stem polypeptides.

107. The carrier-formulated mRNA according to claim 105, wherein the two or more coding sequences that encode influenza HA stem polypeptides are derived from influenza A Group 1 and/or influenza A Group 2.

108. The carrier-formulated mRNA according to claim 106, wherein the two or more coding sequences that encode influenza HA stem polypeptides are derived from influenza A Group 1 and/or influenza A Group 2.

109. The carrier-formulated mRNA according to claim 107, wherein at least one of the two or more coding sequence that encodes an influenza HA stem polypeptide derived from influenza A Group 1 and influenza A Group 2.

110. The carrier-formulated mRNA according to claim 109, wherein influenza A Group 1 comprises:
(i) subtype H1, H2, H5, H6, H8, H9, H11, H12, H13, H16, H17 and/or H18; or
(ii) subtype H1.

111. The carrier-formulated mRNA according to claim 109, wherein influenza A Group 2 comprises:
(i) subtype H3, H4, H7, H10, H14 and/or H15;
(ii) H3 or H10; or
(iii) H10.

112. The carrier-formulated mRNA according to claim 109, wherein at least one of said two or more coding sequence that encodes an influenza HA stem polypeptide derived influenza A subtype H1 and at least one of said two or more coding sequence that encodes an influenza HA stem polypeptide derived from influenza A subtype H10.

113. The carrier-formulated mRNA according to claim 95, wherein the mRNA comprises at least one chemical modification selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

114. The carrier-formulated mRNA, according to claim 95, wherein the mRNA is self-replicating.

115. An immunogenic composition comprising the carrier-formulated mRNA according to claim 95, wherein the composition comprises at least one pharmaceutically acceptable carrier.

116. A vaccine comprising the mRNA of claim 95 and/or the immunogenic composition of claim 15.

117. A kit or kit of parts comprising the RNA of claim 95, and/or the composition of claim 115, and/or the vaccine of claim 116.

118. A method of treating or preventing a disorder, wherein the method comprises applying or administering to a subject in need thereof the carrier-formulated mRNA of claim 95, the composition of claim 115, the vaccine of claim 116, or the kit or kit of parts of claim 117.

119. A method of eliciting an immune response, wherein the method comprises applying or administering to a subject in need thereof the carrier-formulated mRNA of claim 95, the composition of claim 115, the vaccine of claim 116, or the kit or kit of parts of claim 117.


SEQUENCE LISTING (24 sequences)

SEQ ID NO: 1 - protein, 198 aa, Influenza A virus
DTICIGYHANNSTDTVDTVLEKNVTVTHSVNLGSGLRMVTGLRNIPQRETRGLFGAIAGFIEGGWTGMVDGWYGYHHQNEQGSGYAADQKSTQNAINGITNMVNSVIEKMGSGGSGTDLAELLVLLLNERTLDFHDSNVKNLYEKVKSQLKNNAKEIGNGCFEFYHKCNNECMESVKNGTYDYPKYSEESKLNREKID

SEQ ID NO: 2 - protein, 198 aa, Influenza A virus
DTLCIGYHANNSTDTVDTVLEKNVTVTHSVNLGSGLRLATGLRNVPSIQSRGLFGAIAGFIEGGWTGMVDGWYGYHHQNEQGSGYAADLKSTQNAIDKITNMVNSVIEKMGSGGSGTYNAELLVLLLNERTLDYHDSNVKNLYEKVRNQLKNNAKEIGNGCFEFYHKCDNTCMESVKNGTYDYPKYSEEAKLNREKID

SEQ ID NO: 3 - protein, 212 aa, Influenza A virus
QKLPGNDNSTATLCLGHHAVPNGTIVKTITNDQIEVTNATELVFPGCGVLKLATGMRNVPEKQTRGIFGAIAGFIENGWEGMVDGWYGFRHQNSEGIGQAADLKSTQAAINQINGMVNRVIELMEQGGPDCYLAELLVALLNQHVIDLTDSEMRKLFERTKKQLRENAEDMGNGCFKIYHKCDNACIGSIRNGTYDHDVYRDEALNNRFQIK

SEQ ID NO: 4 - protein, 203 aa, Influenza A virus
DKICLGHHAVANGTIVKTLTNEQEEVTNATELVFPGCGVLMLATGMRNVPELIQGRGLFGAIAGFLENGWEGMVDGWYGFRHQNAQGTGQAADYKSTQAAIDQITGMVNRVVELMEQGGPDCYLAELLVAMLNQHVIDMADSEMRNLYERVRKQLRQNAEEDGKGCFEIYHACDDSCMESIRNNTYDHSQYREEALLNRLNIN

SEQ ID NO: 5 - protein, 163 aa, Helicobacter pylori
DIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 6 - protein, 383 aa, Artificial Sequence
  Polypeptide sequence of H1ssF_pylori (signal peptide-stabilised HA stem from A/New Caledonia/20/1999 (H1N1)-SGG-H. pylori ferritin)
MKAKLLVLLCTFTATYADTICIGYHANNSTDTVDTVLEKNVTVTHSVNLGSGLRMVTGLRNIPQRETRGLFGAIAGFIEGGWTGMVDGWYGYHHQNEQGSGYAADQKSTQNAINGITNMVNSVIEKMGSGGSGTDLAELLVLLLNERTLDFHDSNVKNLYEKVKSQLKNNAKEIGNGCFEFYHKCNNECMESVKNGTYDYPKYSEESKLNREKIDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 7 - protein, 381 aa, Artificial Sequence
  Polypeptide sequence of H1ssF_pylori (signal peptide-stabilised HA stem from A/Michigan/45/2015 (H1N1)-SGG-H. pylori ferritin)
MKAILVVLLYTFTTANADTLCIGYHANNSTDTVDTVLEKNVTVTHSVNLGSGLRLATGLRNVPSIQSRGLFGAIAGFIEGGWTGMVDGWYGYHHQNEQGSGYAADLKSTQNAIDKITNMVNSVIEKMGSGGSGTYNAELLVLLLNERTLDYHDSNVKNLYEKVRNQLKNNAKEIGNGCFEFYHKCDNTCMESVKNGTYDYPKYSEEAKLNREKIDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 8 - protein, 399 aa, Artificial Sequence
  Polypeptide sequence of H3ssF_pylori (signal peptide-stabilised HA stem from A/Finland/486/2004 (H3N2)-SGG-H. pylori ferritin)
MKTIIALSYILCLVFAQKLPGNDNSTATLCLGHHAVPNGTIVKTITNDQIEVTNATELVFPGCGVLKLATGMRNVPEKQTRGIFGAIAGFIENGWEGMVDGWYGFRHQNSEGIGQAADLKSTQAAINQINGMVNRVIELMEQGGPDCYLAELLVALLNQHVIDLTDSEMRKLFERTKKQLRENAEDMGNGCFKIYHKCDNACIGSIRNGTYDHDVYRDEALNNRFQIKSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGSRKS

SEQ ID NO: 9 - protein, 386 aa, Artificial Sequence
  Polypeptide sequence of H10ssF_pylori (signal peptide-stabilised HA stem from A/Jiangxi/IPB13/2013 (H10N8)-SGG-H. pylori ferritin)
MYKIVVIIALLGAVKGLDKICLGHHAVANGTIVKTLTNEQEEVTNATELVFPGCGVLMLATGMRNVPELIQGRGLFGAIAGFLENGWEGMVDGWYGFRHQNAQGTGQAADYKSTQAAIDQITGMVNRVVELMEQGGPDCYLAELLVAMLNQHVIDMADSEMRNLYERVRKQLRQNAEEDGKGCFEIYHACDDSCMESIRNNTYDHSQYREEALLNRLNINSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 10 - protein, 450 aa, Artificial Sequence
  Polypeptide sequence of insect ferritin (iFH-F2A-iFL-6R; single polypeptide; insect ferritin heavy chain-self-cleaving construct-insect ferritin light chain)
MDSKGSSQKGSRLLLLLVVSNLLLPQGVVARSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDVRRRRRRSGSGAPVKQTLNFDLLKLAGDVESNPGPMDSKGSSQKGSRLLLLLVVSNLLLPQGVVAEYGSHGNVATELQAYAKLHLERSYDYLLSAAYFNNYQTNRAGFSKLFKKLSDEAWSKTIDIIKHVTKRGDKMNFDQHSTMKTERKNYTAENHELEALAKALDTQKELAERAFYIHREATRNSQHLHDPEIAQYLEEEFIEDHAEKIRTLAGHTSDLKKFITANNGHDLSLALYVFDEYLQKTV

SEQ ID NO: 11 - protein, 830 aa, Artificial Sequence
  Polypeptide sequence of H1ss_iH-F2A-H1ss_IL-6R (single polypeptide; self-cleaving construct)
MKAILVVLLYTFTTANADTLCIGYHANNSTDTVDTVLEKNVTVTHSVNLGSGLRLATGLRNVPSIQSRGLFGAIAGFIEGGWTGMVDGWYGYHHQNEQGSGYAADLKSTQNAIDKITNMVNSVIEKMGSGGSGTYNAELLVLLLNERTLDYHDSNVKNLYEKVRNQLKNNAKEIGNGCFEFYHKCDNTCMESVKNGTYDYPKYSEEAKLNREKIDGGSGGRSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDVRRRRRRSGSGAPVKQTLNFDLLKLAGDVESNPGPMKAILVVLLYTFTTANADTLCIGYHANNSTDTVDTVLEKNVTVTHSVNLGSGLRLATGLRNVPSIQSRGLFGAIAGFIEGGWTGMVDGWYGYHHQNEQGSGYAADLKSTQNAIDKITNMVNSVIEKMGSGGSGTYNAELLVLLLNERTLDYHDSNVKNLYEKVRNQLKNNAKEIGNGCFEFYHKCDNTCMESVKNGTYDYPKYSEEAKLNREKIDGGSGGEYGSHGNVATELQAYAKLHLERSYDYLLSAAYFNNYQTNRAGFSKLFKKLSDEAWSKTIDIIKHVTKRGDKMNFDQHSTMKTERKNYTAENHELEALAKALDTQKELAERAFYIHREATRNSQHLHDPEIAQYLEEEFIEDHAEKIRTLAGHTSDLKKFITANNGHDLSLALYVFDEYLQKTV

SEQ ID NO: 12 - protein, 856 aa, Artificial Sequence
  Polypeptide sequence of H3ss_iH-F2A-H3ss_IL-6R (single polypeptide; self-cleaving construct)
MKTIIALSYILCLVFAQKLPGNDNSTATLCLGHHAVPNGTIVKTITNDQIEVTNATELVFPGCGVLKLATGMRNVPEKQTRGIFGAIAGFIENGWEGMVDGWYGFRHQNSEGIGQAADLKSTQAAINQINGMVNRVIELMEQGGPDCYLAELLVALLNQHVIDLTDSEMRKLFERTKKQLRENAEDMGNGCFKIYHKCDNACIGSIRNGTYDHDVYRDEALNNRFQIKGGSGGRSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDVRRRRRRSGSGAPVKQTLNFDLLKLAGDVESNPGPMKTIIALSYILCLVFAQKLPGNDNSTATLCLGHHAVPNGTIVKTITNDQIEVTNATELVFPGCGVLKLATGMRNVPEKQTRGIFGAIAGFIENGWEGMVDGWYGFRHQNSEGIGQAADLKSTQAAINQINGMVNRVIELMEQGGPDCYLAELLVALLNQHVIDLTDSEMRKLFERTKKQLRENAEDMGNGCFKIYHKCDNACIGSIRNGTYDHDVYRDEALNNRFQIKGGSGGEYGSHGNVATELQAYAKLHLERSYDYLLSAAYFNNYQTNRAGFSKLFKKLSDEAWSKTIDIIKHVTKRGDKMNFDQHSTMKTERKNYTAENHELEALAKALDTQKELAERAFYIHREATRNSQHLHDPEIAQYLEEEFIEDHAEKIRTLAGHTSDLKKFITANNGHDLSLALYVFDEYLQKTV

SEQ ID NO: 13 - protein, 840 aa, Artificial Sequence
  Polypeptide sequence of H10ss_iH-F2A-H10ss_IL-6R (single polypeptide; self-cleaving construct)
MYKIVVIIALLGAVKGLDKICLGHHAVANGTIVKTLTNEQEEVTNATELVFPGCGVLMLATGMRNVPELIQGRGLFGAIAGFLENGWEGMVDGWYGFRHQNAQGTGQAADYKSTQAAIDQITGMVNRVVELMEQGGPDCYLAELLVAMLNQHVIDMADSEMRNLYERVRKQLRQNAEEDGKGCFEIYHACDDSCMESIRNNTYDHSQYREEALLNRLNINGGSGGRSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDVRRRRRRSGSGAPVKQTLNFDLLKLAGDVESNPGPMYKIVVIIALLGAVKGLDKICLGHHAVANGTIVKTLTNEQEEVTNATELVFPGCGVLMLATGMRNVPELIQGRGLFGAIAGFLENGWEGMVDGWYGFRHQNAQGTGQAADYKSTQAAIDQITGMVNRVVELMEQGGPDCYLAELLVAMLNQHVIDMADSEMRNLYERVRKQLRQNAEEDGKGCFEIYHACDDSCMESIRNNTYDHSQYREEALLNRLNINGGSGGEYGSHGNVATELQAYAKLHLERSYDYLLSAAYFNNYQTNRAGFSKLFKKLSDEAWSKTIDIIKHVTKRGDKMNFDQHSTMKTERKNYTAENHELEALAKALDTQKELAERAFYIHREATRNSQHLHDPEIAQYLEEEFIEDHAEKIRTLAGHTSDLKKFITANNGHDLSLALYVFDEYLQKTV

SEQ ID NO: 14 - protein, 843 aa, Artificial Sequence
  Polypeptide sequence of H1ss_iH-F2A-H3ss_iL-6R (H1/H3 ssF insect - single polypeptide; self-cleaving construct)
MKAILVVLLYTFTTANADTLCIGYHANNSTDTVDTVLEKNVTVTHSVNLGSGLRLATGLRNVPSIQSRGLFGAIAGFIEGGWTGMVDGWYGYHHQNEQGSGYAADLKSTQNAIDKITNMVNSVIEKMGSGGSGTYNAELLVLLLNERTLDYHDSNVKNLYEKVRNQLKNNAKEIGNGCFEFYHKCDNTCMESVKNGTYDYPKYSEEAKLNREKIDGGSGGRSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDVRRRRRRSGSGAPVKQTLNFDLLKLAGDVESNPGPMKTIIALSYILCLVFAQKLPGNDNSTATLCLGHHAVPNGTIVKTITNDQIEVTNATELVFPGCGVLKLATGMRNVPEKQTRGIFGAIAGFIENGWEGMVDGWYGFRHQNSEGIGQAADLKSTQAAINQINGMVNRVIELMEQGGPDCYLAELLVALLNQHVIDLTDSEMRKLFERTKKQLRENAEDMGNGCFKIYHKCDNACIGSIRNGTYDHDVYRDEALNNRFQIKGGSGGEYGSHGNVATELQAYAKLHLERSYDYLLSAAYFNNYQTNRAGFSKLFKKLSDEAWSKTIDIIKHVTKRGDKMNFDQHSTMKTERKNYTAENHELEALAKALDTQKELAERAFYIHREATRNSQHLHDPEIAQYLEEEFIEDHAEKIRTLAGHTSDLKKFITANNGHDLSLALYVFDEYLQKTV

SEQ ID NO: 15 - protein, 835 aa, Artificial Sequence
  Polypeptide sequence of H1ss_iH-F2A-H10ss_iL-6R (H1/H10 ssF insect - single polypeptide; self-cleaving construct)
MKAILVVLLYTFTTANADTLCIGYHANNSTDTVDTVLEKNVTVTHSVNLGSGLRLATGLRNVPSIQSRGLFGAIAGFIEGGWTGMVDGWYGYHHQNEQGSGYAADLKSTQNAIDKITNMVNSVIEKMGSGGSGTYNAELLVLLLNERTLDYHDSNVKNLYEKVRNQLKNNAKEIGNGCFEFYHKCDNTCMESVKNGTYDYPKYSEEAKLNREKIDGGSGGRSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDVRRRRRRSGSGAPVKQTLNFDLLKLAGDVESNPGPMYKIVVIIALLGAVKGLDKICLGHHAVANGTIVKTLTNEQEEVTNATELVFPGCGVLMLATGMRNVPELIQGRGLFGAIAGFLENGWEGMVDGWYGFRHQNAQGTGQAADYKSTQAAIDQITGMVNRVVELMEQGGPDCYLAELLVAMLNQHVIDMADSEMRNLYERVRKQLRQNAEEDGKGCFEIYHACDDSCMESIRNNTYDHSQYREEALLNRLNINGGSGGEYGSHGNVATELQAYAKLHLERSYDYLLSAAYFNNYQTNRAGFSKLFKKLSDEAWSKTIDIIKHVTKRGDKMNFDQHSTMKTERKNYTAENHELEALAKALDTQKELAERAFYIHREATRNSQHLHDPEIAQYLEEEFIEDHAEKIRTLAGHTSDLKKFITANNGHDLSLALYVFDEYLQKTV

SEQ ID NO: 16 - protein, 835 aa, Artificial Sequence
  Polypeptide sequence of H1NC99ss_iH-F2A-H10ss_iL-6R (H1NC99/H10 ssF insect - single polypeptide; self-cleaving construct)
MKAKLLVLLCTFTATYADTICIGYHANNSTDTVDTVLEKNVTVTHSVNLGSGLRMVTGLRNIPQRETRGLFGAIAGFIEGGWTGMVDGWYGYHHQNEQGSGYAADQKSTQNAINGITNMVNSVIEKMGSGGSGTDLAELLVLLLNERTLDFHDSNVKNLYEKVKSQLKNNAKEIGNGCFEFYHKCNNECMESVKNGTYDYPKYSEESKLNREKIDGGSGGRSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDVRRRRRRSGSGAPVKQTLNFDLLKLAGDVESNPGPMYKIVVIIALLGAVKGLDKICLGHHAVANGTIVKTLTNEQEEVTNATELVFPGCGVLMLATGMRNVPELIQGRGLFGAIAGFLENGWEGMVDGWYGFRHQNAQGTGQAADYKSTQAAIDQITGMVNRVVELMEQGGPDCYLAELLVAMLNQHVIDMADSEMRNLYERVRKQLRQNAEEDGKGCFEIYHACDDSCMESIRNNTYDHSQYREEALLNRLNINGGSGGEYGSHGNVATELQAYAKLHLERSYDYLLSAAYFNNYQTNRAGFSKLFKKLSDEAWSKTIDIIKHVTKRGDKMNFDQHSTMKTERKNYTAENHELEALAKALDTQKELAERAFYIHREATRNSQHLHDPEIAQYLEEEFIEDHAEKIRTLAGHTSDLKKFITANNGHDLSLALYVFDEYLQKTV

SEQ ID NO: 17 - protein, 173 aa, Trichoplusia ni
RSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDV

SEQ ID NO: 18 - protein, 183 aa, Trichoplusia ni
EYGSHGNVATELQAYAKLHLERSYDYLLSAAYFNNYQTNRAGFSKLFKKLSDEAWSKTIDIIKHVTKRGDKMNFDQHSTMKTERKNYTAENHELEALAKALDTQKELAERAFYIHREATRNSQHLHDPEIAQYLEEEFIEDHAEKIRTLAGHTSDLKKFITANNGHDLSLALYVFDEYLQKTV

SEQ ID NO: 19 - protein, 5 aa, Artificial Sequence
  Linker sequence
GGSGG

SEQ ID NO: 20 - RNA, 8889 nt, Artificial Sequence
  Nucleic acid sequence of H1ssF_pylori (from A/Michigan/45/2015 (H1N1))
gauaggcggcgcaugagagaagcccagaccaauuaccuacccaaaauggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcucgccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgagaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaagccgaugucgacuugauguuacaagaggcuggggccggcucaguggagacaccucguggcuugauaaagguuaccagcuacgauggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagaacuagucacugggcuagggcucacaggcgagcugguggauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaagcuuuugcuugucaugcagguacucucagagcgcucauagccauuauaagaccuaaaaaggcagugcucugcggggaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacuggaagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuuagcauguugaccaagaaagcuugucugcaucugaaucccggcggaaccugugucagcauagguuaugguuacgcugacagggccagcgaaagcaucauuggugcuauagcgcggcaguucaaguuuucccggguaugcaaaccgaaauccucacuugaagagacggaaguucuguuuguauucauuggguacgaucgcaaggcccguacgcacaauccuuacaagcuuucaucaaccuugaccaacauuuauacagguuccagacuccacgaagccggaugugcacccucauaucauguggugcgaggggauauugccacggccaccgaaggagugauuauaaaugcugcuaacagcaaaggacaaccuggcggaggggugugcggagcgcuguauaagaaauucccggaaagcuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgacucuucagugacagaaccugaugcagagcuggugagggugcauccgaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaagagucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacuggugugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaggaagaagaggauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcucgagaaccagccuaguuuccaccccgccaggcgugaauagggugaucacuagagaggagcucgaggcgcuuaccccgucacgcacuccuagcaggucggucucgagaaccagccuggucuccaacccgccaggcguaaauagggugauuacaagagaggaguuugaggcguucguagcacaacaacaaugacgguuugaugcgggugcauacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuuggcaaaccucuggcagcagacgaugaacaugaugaugacaggagaagggcauugcaugaagagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggcuaaccugaauggacuacgacauagucuaguccgccaagaugaaagccauucugguggugcugcucuacaccuucacaacagccaacgcugacacacugugcaucggcuaucacgccaacaacuccaccgacaccguggauaccgugcuggagaagaacgugacagugacccacagcgucaaccucggaagcggccucagacucgcuaccggacugagaaaugugcccagcauccaaucuaggggacuguuuggagccaucgcuggauucaucgaaggcggcuggaccggcauggucgacggcugguacggcuaccaucaucagaacgaacaaggcagcggcuaugccgcugaucugaaauccacccagaacgccaucgacaagaucacaaacauggugaacagcgugaucgagaagaugggcagcggaggcagcggcaccuacaacgcugagcugcuggugcugcugcugaacgagagaacacuggacuaccacgacuccaacgucaagaaccucuaugagaaggugagaaaccagcugaagaacaacgccaaggagaucggaaacggcugcuucgaguucuaccacaagugcgacaauacaugcauggaguccgucaagaacggcaccuacgacuauccuaaguacuccgaggaggccaaacugaauagagagaaaaucgacuccggaggcgacaucaucaagcugcucaacgagcaagugaacaaggagaugcagagcuccaaucuguacauguccaugagcagcugguguuacacccauucucuggauggagcuggacuguuucuguucgaccaugcugccgaggaguaugagcacgccaagaaacugauuaucuuccucaacgagaacaacgugcccguccagcugacaagcaucuccgcccccgaacacaaguuugagggccucacccagaucuuucagaaagccuaugagcacgagcagcacaucagcgaguccaucaauaacaucguggaccacgccaucaaguccaaggaccacgcuaccuucaacuuucugcagugguacguggccgagcaacacgaggaggagguccucuuuaaggacauucuggacaagaucgaacugaucggaaacgagaaccacggccucuaccucgccgaccaguaugucaagggcaucgccaaguccagaaagagcugaugaggcgcgcccacccagcggccgcauacagcagcaauuggcaagcugcuuacauagaacucgcggcgauuggcaugccgccuuaaaauuuuuauuuuauuuuucuuuucuuuuccgaaucggauuuuguuuuuaauauuucaaaaaaaaaaaaaaaaaaaaaaaaaaaaaaaaaaaaaaa

SEQ ID NO: 21 - RNA, 8904 nt, Artificial Sequence
  Nucleic acid sequence of H10ssF_pylori (from A/Jiangxi/IPB13/2013 (H10N8))
gauaggcggcgcaugagagaagcccagaccaauuaccuacccaaaauggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcucgccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgagaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaagccgaugucgacuugauguuacaagaggcuggggccggcucaguggagacaccucguggcuugauaaagguuaccagcuacgauggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagaacuagucacugggcuagggcucacaggcgagcugguggauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaagcuuuugcuugucaugcagguacucucagagcgcucauagccauuauaagaccuaaaaaggcagugcucugcggggaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacuggaagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuuagcauguugaccaagaaagcuugucugcaucugaaucccggcggaaccugugucagcauagguuaugguuacgcugacagggccagcgaaagcaucauuggugcuauagcgcggcaguucaaguuuucccggguaugcaaaccgaaauccucacuugaagagacggaaguucuguuuguauucauuggguacgaucgcaaggcccguacgcacaauccuuacaagcuuucaucaaccuugaccaacauuuauacagguuccagacuccacgaagccggaugugcacccucauaucauguggugcgaggggauauugccacggccaccgaaggagugauuauaaaugcugcuaacagcaaaggacaaccuggcggaggggugugcggagcgcuguauaagaaauucccggaaagcuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgacucuucagugacagaaccugaugcagagcuggugagggugcauccgaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaagagucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacuggugugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaggaagaagaggauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcucgagaaccagccuaguuuccaccccgccaggcgugaauagggugaucacuagagaggagcucgaggcgcuuaccccgucacgcacuccuagcaggucggucucgagaaccagccuggucuccaacccgccaggcguaaauagggugauuacaagagaggaguuugaggcguucguagcacaacaacaaugacgguuugaugcgggugcauacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuuggcaaaccucuggcagcagacgaugaacaugaugaugacaggagaagggcauugcaugaagagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggcuaaccugaauggacuacgacauagucuaguccgccaagauguacaaaauuguggucaucaucgcucugcugggcgcugugaaaggacuggauaagaucugucugggccaucaugcugucgccaacggcaccaucgucaagacacugaccaaugagcaagaggaagucaccaacgccaccgagcugguguuccccggcugcggagugcugaugcuggccaccggaaugaggaacgugcccgaacugauccaaggaagaggacuguuuggagccauugccggauuccucgagaacggcugggagggcaugguggauggcugguacggauuuagacaucagaacgcccaaggcaccggacaagccgccgacuacaaguccacacaagccgcuaucgaccagaucaccggaauggugaacagaguggucgagcugauggaacaaggaggacccgauugcuaccucgcugaacuccucguggccaugcucaaccaacacgucauugacauggccgacagcgagaugaggaaucuguacgagagagugagaaagcagcucagacagaacgccgaagaggacggaaagggcugcuucgagaucuaccacgcuugcgacgacagcuguauggagagcauuagaaacaauaccuacgaccacucccaguauagagaggaggcucugcucaauagacugaacaucaacuccggcggcgacaucaucaagcugcugaaugagcaagugaacaaggagaugcagagcagcaaccucuacaugagcaugagcagcuggugcuauacccauucucuggacggagccggacuguuucuguuugaccaugccgcugaggaguacgaacacgccaagaagcugaucauuuuucugaacgagaacaacguccccgugcagcugaccagcauuuccgcccccgagcacaaguuugaaggccucacccagaucuuccagaaagccuacgagcacgagcagcacauuuccgagagcaucaacaauaucguggaccacgccaucaaguccaaggaccacgccaccuuuaacuuucugcaaugguacguggccgagcagcacgaggaggaggugcucuuuaaagacauccucgacaagaucgagcugaucggcaacgaaaaccauggacuguaucuggcugaccaguacgugaagggaaucgccaaguccagaaaguccugaugaggcgcgcccacccagcggccgcauacagcagcaauuggcaagcugcuuacauagaacucgcggcgauuggcaugccgccuuaaaauuuuuauuuuauuuuucuuuucuuuuccgaaucggauuuuguuuuuaauauuucaaaaaaaaaaaaaaaaaaaaaaaaaaaaaaaaaaaaaaa

SEQ ID NO: 22 - RNA, 8895 nt, Artificial Sequence
  Nucleic acid sequence of H1NC99ssF_pylori (from A/New Caledonia/20/1999 (H1N1))
gauaggcggcgcaugagagaagcccagaccaauuaccuacccaaaauggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcucgccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgagaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaagccgaugucgacuugauguuacaagaggcuggggccggcucaguggagacaccucguggcuugauaaagguuaccagcuacgauggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagaacuagucacugggcuagggcucacaggcgagcugguggauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaagcuuuugcuugucaugcagguacucucagagcgcucauagccauuauaagaccuaaaaaggcagugcucugcggggaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacuggaagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuuagcauguugaccaagaaagcuugucugcaucugaaucccggcggaaccugugucagcauagguuaugguuacgcugacagggccagcgaaagcaucauuggugcuauagcgcggcaguucaaguuuucccggguaugcaaaccgaaauccucacuugaagagacggaaguucuguuuguauucauuggguacgaucgcaaggcccguacgcacaauccuuacaagcuuucaucaaccuugaccaacauuuauacagguuccagacuccacgaagccggaugugcacccucauaucauguggugcgaggggauauugccacggccaccgaaggagugauuauaaaugcugcuaacagcaaaggacaaccuggcggaggggugugcggagcgcuguauaagaaauucccggaaagcuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgacucuucagugacagaaccugaugcagagcuggugagggugcauccgaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaagagucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacuggugugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaggaagaagaggauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcucgagaaccagccuaguuuccaccccgccaggcgugaauagggugaucacuagagaggagcucgaggcgcuuaccccgucacgcacuccuagcaggucggucucgagaaccagccuggucuccaacccgccaggcguaaauagggugauuacaagagaggaguuugaggcguucguagcacaacaacaaugacgguuugaugcgggugcauacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuuggcaaaccucuggcagcagacgaugaacaugaugaugacaggagaagggcauugcaugaagagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggcuaaccugaauggacuacgacauagucuaguccgccaagaugaaggccaagcugcuggugcugcuguguaccuucaccgccaccuacgccgacaccaucugcaucggcuaucacgccaacaauuccaccgauaccgucgacacagugcuggagaagaacgugaccgucacccacagcgugaaucugggcagcggacugagaauggugaccggacugaggaacauuccucagagagagacaagaggacuguuuggcgcuaucgccggcuucaucgagggcggauggaccggcaugguggauggcugguacggcuaccaccaucaaaacgagcaaggcuccggcuaugccgccgaucagaaaagcacccagaacgccaucaacggcaucaccaauauggugaacuccgugaucgagaagaugggcuccggaggaagcggcacagaucuggcugagcugcuggugcugcugcucaaugagagaacccucgacuuucacgacuccaacgucaagaaucuguacgagaaggugaagucccagcugaagaacaacgccaaggagaucggcaacggcugcuucgaguucuaccacaagugcaacaacgagugcauggaguccgugaaaaacggcaccuacgacuaccccaaguauagcgaggagagcaagcugaauagagagaagaucgacuccggcggcgacaucaucaagcugcucaacgagcaagugaacaaggagaugcagagcuccaaucuguacauguccauguccagcuggugcuacacacacucucuggauggcgccggacuguuucuguuugaccacgccgccgaagaguacgagcacgccaagaagcucaucaucuuccucaaugagaacaacguccccgugcagcugaccagcaucuccgcccccgagcauaaguuugagggccucacccagaucuuccagaaggccuacgagcacgagcagcacaucuccgagagcaucaacaacaucgucgaccacgccaucaaguccaaggaccacgcuaccuucaauuuucugcagugguacgucgccgagcagcacgaggaagaggugcuguucaaggacauucuggacaagaucgagcugaucggcaacgagaaccacggacuguaucuggccgaucaguacgugaaaggcaucgccaagucuagaaagagcggauccugaugaggcgcgcccacccagcggccgcauacagcagcaauuggcaagcugcuuacauagaacucgcggcgauuggcaugccgccuuaaaauuuuuauuuuauuuuucuuuucuuuuccgaaucggauuuuguuuuuaauauuucaaaaaaaaaaaaaaaaaaaaaaaaaaaaaaaaaaaaaaa

SEQ ID NO: 23 - RNA, 10251 nt, Artificial Sequence
  Nucleic acid sequence of H1ss_iH-F2A-H10_iL-6R (from A/Michigan/45/2015 (H1N1) and  A/Jiangxi/IPB13/2013 (H10N8), respectively)
gauaggcggcgcaugagagaagcccagaccaauuaccuacccaaaauggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcucgccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgagaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaagccgaugucgacuugauguuacaagaggcuggggccggcucaguggagacaccucguggcuugauaaagguuaccagcuacgauggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagaacuagucacugggcuagggcucacaggcgagcugguggauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaagcuuuugcuugucaugcagguacucucagagcgcucauagccauuauaagaccuaaaaaggcagugcucugcggggaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacuggaagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuuagcauguugaccaagaaagcuugucugcaucugaaucccggcggaaccugugucagcauagguuaugguuacgcugacagggccagcgaaagcaucauuggugcuauagcgcggcaguucaaguuuucccggguaugcaaaccgaaauccucacuugaagagacggaaguucuguuuguauucauuggguacgaucgcaaggcccguacgcacaauccuuacaagcuuucaucaaccuugaccaacauuuauacagguuccagacuccacgaagccggaugugcacccucauaucauguggugcgaggggauauugccacggccaccgaaggagugauuauaaaugcugcuaacagcaaaggacaaccuggcggaggggugugcggagcgcuguauaagaaauucccggaaagcuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgacucuucagugacagaaccugaugcagagcuggugagggugcauccgaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaagagucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacuggugugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaggaagaagaggauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcucgagaaccagccuaguuuccaccccgccaggcgugaauagggugaucacuagagaggagcucgaggcgcuuaccccgucacgcacuccuagcaggucggucucgagaaccagccuggucuccaacccgccaggcguaaauagggugauuacaagagaggaguuugaggcguucguagcacaacaacaaugacgguuugaugcgggugcauacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuuggcaaaccucuggcagcagacgaugaacaugaugaugacaggagaagggcauugcaugaagagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggcuaaccugaauggacuacgacauagucuaguccgccaagaugaaggcuauucucgucgugcugcuguacaccuucaccacagccaaugccgauacacugugcauuggauaccaugccaacaauagcaccgauaccguggauacaguccucgagaagaacgugaccgucacccacagcgucaaccucggcagcggacugagacuggccaccggccucagaaacgugccuuccauccaauccagaggacuguuuggagccaucgcuggcuuuaucgagggaggauggaccggcaugguggacggcugguauggcuaccaccaucagaacgagcaaggaagcggauacgccgcugaucugaaaagcacccagaacgccaucgacaaaaucaccaauauggucaauuccgucauugagaagaugggcagcggaggaagcggcacauacaacgcugagcugcuggugcugcugcugaacgagaggacacuggauuaucacgacuccaaugucaagaaucuguaugaaaaggugagaaaucaacucaagaacaacgccaaagaaaucggcaacggaugcuucgaguucuaccacaagugcgauaacacaugcauggaguccgugaaaaauggcaccuaugacuaucccaaguacuccgaggaagccaagcugaauagagagaagaucgauggaggcuccggaggaaggagcugcagaaacuccaugaggcagcagauccagauggaagugggagccucucugcaguaccucgccaugggagcccacuucagcaaagacguggugaacagacccggcuucgcccagcuguucuucgaugccgccagcgaggagagagagcaugccaugaagcucaucgaguaucugcugaugaggggcgaacugaccaaugacgugagcucucugcugcaagugagaccucccaccagauccagcuggaaaggaggcguggaggcucuggagcacgcucuguccauggaguccgaugugacaaagagcauuagaaaugugaucaaggcuugcgaggacgacuccgaguucaacgacuaccaucuggucgauuaucucaccggagacuuccucgaagaacaguacaagggccagagggaucuggcuggaaaggccagcacacugaaaaaacugauggauagacaugaggcucugggcgaguuuaucuucgacaagaagcuccucggaaucgacgucagaaggaggagaaggagauccggcagcggagcccccgugaagcaaacacugaacuucgaucugcugaagcuggcuggagacguggaauccaaccccggccccauguauaaaaucguggucaucauugcucugcucggagccgucaagggacuggauaagauuugucucggacaccacgcuguggccaacggaaccaucgucaagacacugaccaacgagcaagaggaggugacaaaugccaccgaacugguguuucccggaugcggagugcugaugcuggcuaccggcaugaggaacguccccgagcugauccaaggcagaggacuguuuggcgcuaucgccggcuuucuggagaauggaugggaaggcauggucgauggcugguacggauuuagacaccagaacgcccaaggcaccggacaagccgccgauuauaaguccacccaagccgcuaucgaccaaaucaccggcauggugaauagagucguggagcugauggagcaaggcggacccgacuguuaucuggcugagcugcuggucgcuaugcucaaccagcacgugaucgacauggcugauuccgagaugaggaaucuguacgaaagggugagaaagcaacugaggcagaacgccgaggaagauggaaaaggcugcuuugagaucuaccacgcuugugacgacagcuguauggaauccaucagaaauaacaccuacgaccacucccaguauagggaggaggcccuccucaacagacugaauaucaacggcggcagcggcggcgaauacggcagccauggcaaugucgcuacagagcugcaagccuacgccaagcugcaucuggaaagauccuacgacuaucugcuguccgccgccuacuuuaacaacuaccagacaaauagagccggauucagcaagcuguucaagaagcugagcgacgaggcuuggagcaaaaccauugacaucaucaagcacgugaccaaaaggggcgacaagaugaacuuugaccagcacagcaccaugaagaccgagaggaagaacuacaccgccgaaaaucacgagcuggaagcucucgccaaggcucuggacacccagaaagaacuggcugagagggccuucuauauccauagggaggccacaagaaacucccagcaucugcacgaccccgagaucgcucaguaucucgaggaggaguucaucgaagaccaugccgaaaagauuaggacacuggccggacacaccagcgaucucaagaaauucaucaccgccaacaacggccacgaucucucucuggcucuguaugucuucgacgaguaucugcagaaaaccgucugaugaggcgcgcccacccagcggccgcauacagcagcaauuggcaagcugcuuacauagaacucgcggcgauuggcaugccgccuuaaaauuuuuauuuuauuuuucuuuucuuuuccgaaucggauuuuguuuuuaauauuucaaaaaaaaaaaaaaaaaaaaaaaaaaaaaaaaaaaaaaa

SEQ ID NO: 24 - RNA, 10251 nt, Artificial Sequence
  Nucleic acid sequence of H1NC99ss_iH-F2A-H10_iL-6R ((from A/New Caledonia/20/1999 (H1N1) and A/Jiangxi/IPB13/2013 (H10N8), respectively)
gauaggcggcgcaugagagaagcccagaccaauuaccuacccaaaauggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcucgccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgagaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaagccgaugucgacuugauguuacaagaggcuggggccggcucaguggagacaccucguggcuugauaaagguuaccagcuacgauggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagaacuagucacugggcuagggcucacaggcgagcugguggauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaagcuuuugcuugucaugcagguacucucagagcgcucauagccauuauaagaccuaaaaaggcagugcucugcggggaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacuggaagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuuagcauguugaccaagaaagcuugucugcaucugaaucccggcggaaccugugucagcauagguuaugguuacgcugacagggccagcgaaagcaucauuggugcuauagcgcggcaguucaaguuuucccggguaugcaaaccgaaauccucacuugaagagacggaaguucuguuuguauucauuggguacgaucgcaaggcccguacgcacaauccuuacaagcuuucaucaaccuugaccaacauuuauacagguuccagacuccacgaagccggaugugcacccucauaucauguggugcgaggggauauugccacggccaccgaaggagugauuauaaaugcugcuaacagcaaaggacaaccuggcggaggggugugcggagcgcuguauaagaaauucccggaaagcuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgacucuucagugacagaaccugaugcagagcuggugagggugcauccgaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaagagucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacuggugugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaggaagaagaggauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcucgagaaccagccuaguuuccaccccgccaggcgugaauagggugaucacuagagaggagcucgaggcgcuuaccccgucacgcacuccuagcaggucggucucgagaaccagccuggucuccaacccgccaggcguaaauagggugauuacaagagaggaguuugaggcguucguagcacaacaacaaugacgguuugaugcgggugcauacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuuggcaaaccucuggcagcagacgaugaacaugaugaugacaggagaagggcauugcaugaagagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggcuaaccugaauggacuacgacauagucuaguccgccaagaugaaagcuaagcugcuggugcugcucuguacauucaccgccaccuaugccgauaccaucugcauuggcuaccacgccaauaacuccacagacaccgucgacaccguccucgagaagaacgugacagugacccauuccgugaaucucggauccggacucagaauggugaccggacugagaaacaucccccaaagggaaaccagaggacuguucggagcuaucgccggauucaucgagggcggcuggaccggcaugguggauggaugguacggcuaccaucaucagaacgaacaaggcagcggcuacgccgcugaucaaaagagcacccagaaugccaucaacggcaucaccaacauggugaacuccgugauugaaaaaaugggcagcggaggcuccggcacagaucuggcugagcugcuggugcugcugcugaaugagagaacacuggacuuccaugacuccaacgucaaaaaucuguacgaaaaggugaagucccagcugaaaaacaacgcuaaagagaucggcaacggcugcuuugaguucuaucacaaauguaacaacgaauguauggaaagcgucaagaacggcacauacgacuaccccaaguacuccgaggaguccaagcucaauagggagaagauugauggcggcuccggcggaaggagcugcagaaacuccaugaggcagcagauccaaauggaagugggcgccucucugcaguaucuggccaugggagcucacuucagcaaagacguggugaauagacccggcuuugcccaacuguucuucgaugccgcuuccgaggaaagagagcacgccaugaagcucauugaguaucugcugaugaggggcgaacugacaaaugacgucuccucucugcugcaagugagaccccccaccagaagcagcuggaagggaggaguggaggcccucgaacacgcucugagcauggagagcgacgugaccaaguccauuagaaacgucaucaaagcuugcgaggacgacuccgaauucaacgacuaccaucugguggacuaucugaccggcgauuuccucgaggagcaguacaagggccaaagggaucuggccggcaaggccagcacacugaagaagcugauggauaggcacgaagcucugggagaguucaucuucgacaagaagcugcucggaaucgacgugagaaggaggaggagaagauccggcagcggcgcucccgugaaacagacccucaacuuugaucugcugaaacuggcuggagauguggaaagcaaccccggacccauguacaaaaucguggugauuauugcucugcugggcgcugucaagggacuggacaaaaucugucugggccaucacgcugucgccaacggcaccaucgucaaaacacugacaaacgagcaagaagaggugaccaacgcuacagagcucguguuccccggcugcggcgugcugaugcuggcuaccggcaugagaaacgugcccgagcucauccaaggaagaggacuguucggcgcuauugccggcuuucuggaaaacggaugggagggcauggucgauggcugguauggcuucagacaucagaaugcccaaggcaccggccaagccgccgacuauaaaagcacccaagccgcuaucgaucagaucaccggcauggugaauagggugguggaacucauggaacaaggcggccccgauuguuaucuggccgaacugcugguggccaugcugaaccagcaugugauugacauggccgacuccgagaugaggaaucucuacgagagggugaggaagcagcugaggcaaaaugcugaagaggacggcaagggcugcuucgagaucuaccaugcuugcgaugauagcugcauggaguccauuaggaauaauaccuacgaccacagccaguauagagaggaggcucugcugaauagacucaacaucaacggcggcuccggaggagaguauggcucccauggcaaugucgcuaccgagcuccaagccuacgcuaaacuccaccucgagagguccuacgacuaucugcucagcgccgccuacuuuaacaacuaccagacaaauagagccggauucagcaagcuguucaagaaacugagcgacgaggcuuggagcaaaaccaucgacauuaucaaacacgugaccaaaaggggcgauaagaugaacuuugaccagcauuccaccaugaagaccgagaggaaaaacuacaccgcugagaaccacgaacucgaagcucucgccaaggcucuggacacccagaaagaacuggcugagagggccuucuauauccauagggaggccacaagaaauagccagcaucugcacgaccccgagaucgcucaguaucucgaggaggaguucaucgaggaccaugccgagaaaauuagaacacuggccggacacaccagcgaucucaagaaauucaucaccgccaacaacggccacgaucucucucuggcucuguaugucuucgacgaguaccuccagaagaccgugugaugaggcgcgcccacccagcggccgcauacagcagcaauuggcaagcugcuuacauagaacucgcggcgauuggcaugccgccuuaaaauuuuuauuuuauuuuucuuuucuuuuccgaaucggauuuuguuuuuaauauuucaaaaaaaaaaaaaaaaaaaaaaaaaaaaaaaaaaaaaaa